(19) United States
(12) Patent Application Publication
(10) Pub. No.: US 2009/0099250 A1
(43) Pub. Date: Apr. 16, 2009

(54) BENZOCYCLOHEPTENE DERIVATIVES AS ESTROGENS HAVING SELECTIVE ACTIVITY

(76) Inventors: Ralf WYRWA, Rothenstein (DE); Olaf Peters, Tabarz (DE); Rolf Bohlmann, Berlin (DE); Peter Droescher, Berlin (DE); Katja Prelle, Berlin (DE); Karl Heinrich Fritzemeier, Berlin (DE); Hans Peter Muhn, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/249,056

(22) Filed: Oct. 10, 2008

Related U.S. Application Data
(60) Provisional application No. 60/979,215, filed on Oct. 11, 2007.

(30) Foreign Application Priority Data
Oct. 11, 2007 (EP) 07 075 893.3

(51) Int. Cl. A61K 31/381; C07C 39/17; A61K 31/122; A61K 31/05; A61P 5/00; C07C 49/747; C07D 333/16

(52) U.S. Cl. 514/438; 568/719; 549/78; 514/681; 568/327; 514/732

(57) ABSTRACT

The present invention describes non-steroidal compounds of the general formula (I)

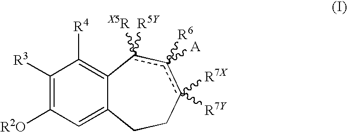

in which
A is

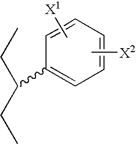

in which
   X¹ means one or more groups on the phenyl ring and represents independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
   X² means one or more groups on the phenyl ring and represents independently of one another an H, halogen, OH, (C₁-C₄)alkyl, (C₁-C₄)alkyl-O, (C₁-C₄)alkenyl, (C₁-C₄)alkynyl, perfluoro-(C₁-C₄)alkyl radical, —CHO or CN, as Estrogens.

This application claims the benefit of the filing date of U.S. Provisional Application Ser. No. 60/979,215 filed Oct. 11, 2007.

### FIELD OF THE INVENTION

The present invention relates to novel compounds of the general formula (I)

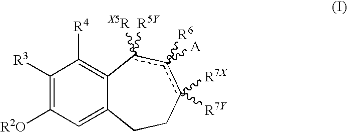

in which
A is

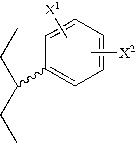

in which
   X¹ means one or more groups on the phenyl ring and represents independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
   X² means one or more groups on the phenyl ring and represents independently of one another an H, halogen, OH, (C₁-C₄)alkyl, (C₁-C₄)alkyl-O, (C₁-C₄)alkenyl, (C₁-C₄)alkynyl, perfluoro-(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H or an F atom;
R⁴ is an H atom, a (C₁-C₄)alkyl, (C₁-C₄)alkenyl, (C₁-C₄)alkynyl, perfluoro-(C₁-C₄)alkyl, silyl(C₁-C₄)alkyl radical, a halogen atom or a nitrile radical;
R^{5X} and R^{5Y} mean independently of one another an H atom, a (C₁-C₄)alkyl, (C₁-C₄)alkenyl, (C₁-C₄)alkynyl, perfluoro(C₁-C₄)alkyl, perfluoro-(C₁-C₄)alkenyl, (C₁-C₄)alkyl-O, aryl, heteroaryl or a CN group, or
R^{5X} and R^{5Y} together are an O atom, an S atom, a ═CHZ in which Z means an H atom, a (C₁-C₄)alkyl, (C₁-C₄)alkenyl radical, or
R^{5X} and R^{5Y} together are a —(C₂-C₄)alkanediyl, (C₂-C₆)alkenediyl radical or —OCH₂— or
R^{5Y} and R⁶ together represent a bond, a —(C₁-C₄)alkanediyl or a (C₂-C₆)alkenediyl radical, and R^{5X} has the meaning given above;
R⁶ means an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical, or
R⁶ and R^{5Y} together represent a bond, a —(C₁-C₄)alkanediyl or a (C₂-C₆)alkenediyl radical, and R^{5X} has the meaning given above, or
R⁶ and R^{7Y} together represent a bond, a —(C₁-C₄)alkanediyl or a (C₂-C₆)alkenediyl radical, and R^{7X} has the meaning given under;
R^{7X} and R^{7Y} represent independently of one another an H atom, a (C₁-C₄)-alkyl, a (C₁-C₄)alkenyl radical, a (C₁-C₄)alkynyl-, or
R^{7X} and R^{7Y} together represent a —(C₂-C₄)alkanediyl, or (C₂-C₆)alkenediyl radical, or
R^{7Y} and R⁶ together represent a bond, a —(C₁-C₄)alkanediyl or a (C₂-C₆)alkenediyl radical, and R^{7X} has the meaning given above, as estrogens.

The present invention further relates to the novel non-steroidal compounds of the general formula (I) as active pharmaceutical ingredients, the preparation thereof, the therapeutic use thereof and pharmaceutical dosage forms comprising the novel compounds.

The novel non-steroidal estrogen receptor modulators of the general formula (I) contained in the present invention are suitable for the treatment of estrogen deficiency-related disorders (age-related, surgery- or medication-related), for the prevention and therapy of inflammatory disorders, for improving fertility and for the therapy of fertility impairments in women, and as component of new types of contraceptives.

### PRIOR ART

### Treatment of Estrogen Deficiency-Related Disorders

Established estrogen therapies for the treatment of hormone deficiency-related symptoms and the protective effect of estrogens on bone, brain, vessel and other organ systems are known as state of the art.

The efficacy of estrogens for the treatment of hormone deficiency-related symptoms such as hot flushes, atrophy of estrogen target organs and incontinence, and successful use of estrogen therapies for preventing loss of bone mass in peri- and post-menopausal women is well-proven and generally accepted (Grady D et al. 1992, Ann Intern Med 117: 1016-1037). There is likewise good documentation of the reduction in the risk of cardiovascular disorders in post-menopausal women or women with ovarian dysfunction of other cause with estrogen replacement therapy compared with women not treated with estrogens (Grady et al., loc. cit.).

Recent investigations additionally demonstrate a protective effect of estrogens in relation to neurodegenerative disorders such as, for example, Alzheimer's disease (Henderson 1997, Neurology 48 (Suppl. 7): p. 27-p. 35; Birge 1997, Neurology 48 (Suppl. 7): p. 6-p. 41; B. R. Bhavnani, Journal of Steroid Biochemistry & Molecular Biology 85 (2003) 473-482), a protective effect on brain functions, such as memory and learning capacity (McEwen et al. 1997, Neurology 48 (Suppl. 7): p. 8-p. 15; Sherwin 1997, Neurology 48 (Suppl. 7): p. 21-p. 26), and in relation to hormone deficiency-related mood fluctuations (Halbreich 1997, Neurology 48 (Suppl. 7): p. 16-p. 20).

Estrogen replacement therapy has further proved to be effective in reducing the incidence of colon carcinoma (Calle E F et al., 1995, J Natl Cancer Inst 87: 517-523).

In conventional estrogen or hormone replacement therapy (HRT), natural estrogens such as oestradiol and conjugated estrogens from horse urine are employed either alone or in combination with a progestogen. Instead of natural estrogens it is also possible to employ derivatives obtained by esterification, such as, for example, 17β-oestradiol valerate.

Because of the stimulating effect of the estrogens used on the endometrium, which leads to an increase in the risk of endometrial carcinoma (Harlap S 1992, Am J Obstet Gynecol 166: 1986-1992), preferably estrogen/progestogen combination products are employed in hormone replacement therapy. The progestational component in the estrogen/progestogen combination avoids hypertrophy of the endometrium, but the progestogen-containing combination is also linked to the occurrence of unwanted irregular bleeding.

Selective estrogens represent a newer alternative to estrogen/progestogen combination products. Selective estrogens have to date been understood to be compounds having estrogen-like effects on brain, bone and vascular system because of their anti-uterotrophic (i.e. anti-estrogenic) partial effect, but not having a proliferative effect on the endometrium.

One class of substances which partly satisfy the desired profile of a selective estrogen are the so-called selective estrogen receptor modulators (SERM) (R. F. Kauffman, H. U. Bryant 1995, DNAP 8 (9): 531-539). These are partial agonists of the estrogen receptor subtype ERa. This type of substances is, however, ineffective in the therapy of acute post-menopausal symptoms such as, for example, hot flushes. One example which may be mentioned of a SERM is raloxifene which has recently been introduced for the indication osteoporosis.

Estrogen receptor modulators with preference for ERbeta, in particular ERbeta-selective agonists, may also have a beneficial effect on brain functions, bladder, intestine and the cardiovascular system without having in the same dose range a hepatic estrogen effect or stimulating effect on endometrium and breast. ERbeta agonists therefore represent a novel option for selective estrogen therapy and for the treatment of hot flushes and mood fluctuations. The occurrence of hot flushes presumably derives from an instability of the hypothalamic thermoregulatory set point caused by the decline in estrogens and the onset of the menopause (Stearns V, Ullmer L, Loepez J F, Smith Y, Isaacs C, Hayes D F (2002) Hot flushes. The Lancet 360: 1851-1861).

ER agonists modulate components of the serotonergic system, and the neurotransmitter serotonin plays an important role in thermoregulation. The modulatory effect of ER agonists on the serotonin receptor system and the serotonin transporter is regarded as essential for stabilizing the thermoregulatory set point. ERbeta is coexpressed with the components of the serotonergic system in areas of the hypothalamus, so that it can be assumed that the modulating effect of ER agonists is mediated by ERbeta. Activation of ERbeta might be sufficient to stabilize the thermoregulatory set point.

### Estrogen Receptor Modulators, Especially ERbeta Agonists, for the Prevention and Therapy of Inflammatory Disorders

It is to be assumed on the basis of recent investigations that ERbeta plays an important regulatory function in inflammatory disorders and disorders of the immune system. This applies in particular to autoimmune diseases such as, for example, rheumatoid arthritis, multiple sclerosis, lupus, Crohn's disease and further inflammatory disorders of the intestine and of the skin such as, for example, psoriasis. ERβ modulation is also relevant in the treatment of endometriosis. On the basis of demonstrations in preclinical models of human inflammatory disorders, it is to be assumed that estrogen receptor modulators, especially ER agonists with ERbeta preference, can be employed for the prevention and treatment of inflammatory disorders (Heather, H. A.; Mol Endocrinol. 2007 January; 21(1):1-13).

### Estrogen Receptor Modulators, in Particular ERbeta Agonists, for the Treatment of Fertility Impairments in Women

The use of novel selective ER modulators (estrogens) likewise opens up new therapeutic possibilities for the treatment of fertility impairments in women, frequently caused by ovarian dysfunction related to surgery, medication or other factors. In vitro fertility treatment is a method which has been established for more than 20 years. Numerous methods for treating ovary-related infertility with exogenous gonadotropins are known. The intention of administration of gonadotropins such as FSH (FSH=follicle-stimulating hormone) is to bring about ovarian stimulation which is intended to make healthy follicle maturation possible.

The follicle is a functional unit of the ovary and has two tasks: it accommodates the oocytes and thus creates the possibility for their growth and maturation. Folliculogenesis comprises the development of an ovarian follicle from the primordial stage to a continuously enlarging antral follicle which represents the last stage before ovulation. Only an optimally developed antral follicle is able to release a mature oocyte by ovulation.

Patients with ovary-related infertility (PCOS=polycystic ovary syndrome) suffer from impaired follicle maturation which is linked both to hormonal and ovulatory impairments and to insufficiently matured oocytes. The number of primary and secondary follicles is in such cases approximately twice as high as in the normal ovary (Hughesden et al., Obstet. Gynecol. Survey 37, 1982, pp. 59-77). There is evidence that the early developmental stages of folliculogenesis (relating to the development from the primordial to the antral follicle) are gonadotropin-independent. It is unclear how large the influence of known paracrine and autocrine factors on early folliculogenesis is (Elvin et al., Mol. Cell Endocrinol. 13, 1999, pp. 1035-1048; McNatty et al., J. Reprod. Fertil. Suppl. 54, 1999, pp. 3-16).

Gonadotropins such as FSH are involved in follicle maturation, i.e. in the development from the early antral follicle to a mature follicle capable of ovulation, mainly in the final developmental stages of folliculogenesis.

Gonadotropins (FSH and anti-estrogens) are preferably used to treat in vivo and in vitro infertility (White et al., J. Clin. Endocrinol. Metab. 81, 1996, pp. 3821-3824). In vitro fertility treatment entails removing oocytes from preovulatory antral follicles and allowing them to mature in vitro to a fertilizable oocyte. After fertilization and pre-embryonic development, one to three embryos are transferred into the woman's uterus.

Treatment with exogenous gonadotropins is accompanied by numerous risks and side effects in many respects. The greatest risk is of overstimulation of the ovaries, and this may in severe cases represent a serious threat to life (OHSS=ovarian hyperstimulation syndrome). Further side effects are the high costs which must be paid by the couples for the in vitro fertility treatment. Negative side effects such as weight gain, bloating, nausea, vomiting and an as yet unknown long-term risk of developing cancer are ascribed to gonadotropin treatment.

One method for avoiding the disadvantages and risks mentioned is regarded as being the induction of maturation and stimulation of follicular growth in ovary-related infertility in vivo with suitable active ingredients, in particular selective ER modulators.

### Estrogen Receptor Beta (ERβ)

The estrogen receptor-β (ERβ) was identified as the second subtype of the estrogen receptor by Kuiper et al. (1996), Proc. Natl. Acad. Sci. 93:5925-5930 and Mosselman, Dijkema (1996) Febs Letters 392: 49-53; Tremblay et al. (1997), Molecular Endocrinology 11: 353-365.

The expression pattern of ERβ differs from that of ERa (Kuiper et al. (1996), Endocrinology 138: 863-870). Thus, ERβ predominates over ERa in the rat prostate, whereas ERα predominates over ERβ in the rat uterus. Areas in which only one of the two ER subtypes is expressed in each case have been identified in the brain (Shugrue et al. (1996), Steroids 61: 678-681; Li et al. (1997), Neuroendocrinology 66:63-67). ERβ is expressed inter alia in areas to which importance for cognitive processes and “mood” is ascribed (Shugrue et al. 1997, J Comparative Neurology 388:507-525).

Further organ systems with comparably high ERβ expression include bone (Onoe Y et al., 1997, Endocrinology 138:4509-4512), the vascular system (Register T C, Adams M R 1998, J Steroid Molec Biol 64: 187-191), the urogenital tract (Kuiper G J M et al. 1997, Endocrinology 138: 863-870), the gastrointestinal tract (Campbell-Thopson 1997, BBRC 240: 478-483), and the testis (Mosselmann S et al. 1996 Febs Lett 392 49-53) including the spermatids (Shugrue et al. 1998, Steroids 63: 498-504). The tissue distribution suggests that estrogens regulate organ functions via ERA. The fact that ERβ is functional in this regard is also evident from investigations on ERa (ERKO) and ERβ (βERKO) knockout mice: in ERKO mice, ovarectomy brings about a loss of bone mass which can be abolished by estrogen replacement (Kimbro et al. 1998, Abstract OR7-4, Endocrine Society Meeting New Orleans). Likewise, in the blood vessels of female ERKO mice, oestradiol inhibits proliferation of the vessel media and smooth muscle cells (lafrati M D et al. 1997, Nature Medicine 3: 545-548). These protective effects of oestradiol in the ERKO mouse presumably take place via ERβ.

Observations on βERKO mice provide evidence of a function of ERβ in the prostate and bladder: symptoms of prostate and bladder hyperplasia occur in older male mice (Krege J H et al. 1998, Proc Natl Acad Sci 95: 15677-15682). In addition, fertility impairments are shown by female (Lubahn D B et al. 1993, Proc Natl Acad Sci 90:11162-11166) and male ERKO mice (Hess R A et al. 1997, Nature 390: 509-512) and female βERKO mice (Krege J H, 1998). This demonstrates the important function of estrogens in relation to maintaining testis and ovary function, and fertility.

Westerlind et al. (1998) describe a differential effect of 16a hydroxyestrone on bone on the one hand, and reproductive organs of the female rat on the other hand (J Bone and Mineral Res 13: 1023-1031).

Our own investigations have revealed that 16a-hydroxyestrone binds three times better to the human estrogen receptor β (ERβ) than to the human estrogen receptor a (ERa). The RBA for the substance on the rat prostate estrogen receptor is five times better than the RBA for the substance on the rat uterus estrogen receptor. The dissociation of the substance described by Westerlind is attributable according to our findings to its preference for ERβ by comparison with ERa.

A selective estrogen effect on certain target organs might be achieved owing to the different tissue and organ distribution of the two ER subtypes by subtype-specific ligands. Substances having preference for ERβ compared with ERa in the in vitro receptor binding assay have been described by Kuiper et al. (1996, Endocrinology 138: 863-870).

The role of selective ERβ ligands is explained further in several publication and reviews such as, for example, in “Estrogen receptor-β: Recent lessons from in vivo studies” Harris H. A., Molecular Endocrinology, 2006, “Benzopyrans are selective Estrogen Receptor-β Agonists with novel activity in models of Benign Prostatic Hyperplasia”, Krishnan V. et al., J. Med. Chem. 2006, 49, 6155-6157 and “Estrogen Receptor beta in Health and Disease”, Gustafsson, J. A. et al., Biology of Reproduction, 2005, 73, 866-871.

### Estrogen Receptor Modulators, in Particular ER Agonists or Estrogens as Components of Oral Contraceptives

Contraceptive methods based on inhibition of ovulation by administration with a combination of an estrogen and a progestogen are very well established. ER modulators are particularly suitable as estrogenic component of combination products for contraception. Target organs of the estrogen in combination products are in particular the pituitary, the ovary and the endometrium. These organs express ERα (Kuiper et al. (1996), Endocrinology 138: 863-870).

### Known Benzocycloheptene Derivatives

WO03/033461 relates to intermediates and a novel process for preparing benzocycloheptene. The process for preparing its intermediates starts from low-cost starting materials, provides the intermediates in high yields and high purity without chromatographic purification steps, and allows preparation on a large scale.

WO00/03979 presents further SERMs with benzocycloheptene basic structure, compounds with potent anti-estrogenic activity. These are selective estrogens with a tissue-selective effect. In particular, the estrogen effect occurs in bone. An advantage of this class of compounds is that an only extremely small or no effect occurs on the uterus and in the liver.

The compounds disclosed in WO00/03979 and WO03/033461 may also have anti-estrogenic activity which can be detected for example in the anti-uterine growth test or in tumour models.

Compounds with such a profile of effects are referred to as selective estrogen receptor modulators (SERMs) as already described previously.

### DISCLOSURE OF THE INVENTION

It is an object of the present invention to provide non-steroidal compounds having an estrogenic effect.

The above object is achieved according to the invention by providing compounds of the general formula (I)

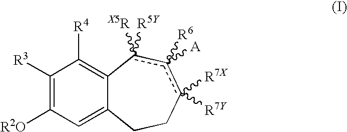

in which
A is

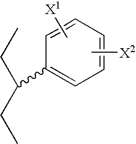

in which
   X¹ is one or more groups on the phenyl ring and is independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
   X² is one or more groups on the phenyl ring and is independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H atom or an F atom;
R⁴ is an H atom, a (C₁-C₄)alkyl, (C₁-C₄)alkenyl, (C₁-C₄)alkynyl, perfluoro(C₁-C₄)alkyl, silyl(C₁-C₄)alkyl radical, a halogen atom or a nitrile radical;
R^{5X} and R^{5Y} are independently of one another an H atom, a (C₁-C₄)alkyl, (C₁-C₄)alkenyl, (C₁-C₄)alkynyl, perfluoro(C₁-C₄)alkyl, perfluoro-(C₁-C₄)alkenyl, (C₁-C₄)alkyl-O, aryl, heteroaryl or a CN group, or
R^{5X} and R^{5Y} together are an O atom, an S atom, a ═CHZ in which Z is an H atom, a (C₁-C₄)alkyl, (C₁-C₄)alkenyl radical, or
R^{5X} and R^{5Y} together are a —(C₂-C₄)alkanediyl, (C₂-C₆)alkenediyl radical or —OCH₂—, or
R^{5Y} and R⁶ together are a bond, a —(C₁-C₄)alkanediyl or a (C₂-C₆)alkenediyl radical, and R^{5X} has the meaning given above;
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical, or
R⁶ and R^{5Y} together are a bond, a —(C₁-C₄)alkanediyl or a (C₂-C₆)alkenediyl radical, and R^{5X} has the meaning given above or
R⁶ and R^{7Y} together are a bond, a —(C₁-C₄)alkanediyl or a (C₂-C₆)alkenediyl radical, and R^{7X} has the meaning given under;
R^{7X} and R^{7Y} are independently of one another an H atom, a (C₁-C₄)-alkyl, a (C₁-C₄)alkenyl radical, a (C₁-C₄)alkynyl-, or
R^{7X} and R^{7Y} together are a —(C₂-C₄)alkanediyl, or (C₂-C₆)alkenediyl radical, or
R^{7Y} and R⁶ together are bond, a —(C₁-C₄)alkanediyl or a (C₂-C₆)alkenediyl radical, and R^{7X} has the meaning given above;
   where the wavy lines on R^{5X}, R^{5Y}, R⁶, R^{7X} and R^{7Y} mean that these substituents may have the α- or β configuration.

Substituents on double bonds may be located cis(Z) or trans(E).

The compounds may be in racemic or enantiopure form.

The compounds according to the invention are suitable as ER modulators for the treatment of estrogen deficiency-related disorders (age-related, related to surgery or medication) for the prevention and therapy of inflammatory disorders, for improving fertility and for the therapy of fertility impairments in women, and as component of novel types of contraceptives.

For example, further embodiments of the compounds of the general formula (I) in the context of the present invention may be defined in dependent claims 2 to 32.

The following compounds of the general formula (I) are preferred according to the present invention:
1) 6-(4-hydroxyphenyl)-8,9-dihydro-7H-benzocyclohepten-2-ol
2) 6-(3-hydroxyphenyl)-8,9-dihydro-7H-benzocyclohepten-2-ol
3) 6-phenyl-8,9-dihydro-7H-benzocyclohepten-2-ol
4) 6-phenyl-5-methyl-8,9-dihydro-7H-benzocyclohepten-2-ol
5) 3-methoxy-9-methyl-8-phenyl-6,7-dihydro-5H-benzocycloheptene
6) 6-phenyl-5-ethyl-8,9-dihydro-7H-benzocyclohepten-2-ol
7) 9-ethyl-3-methoxy-8-phenyl-6,7-dihydro-5H-benzocycloheptene
8) 6-phenyl-5-propyl-8,9-dihydro-7H-benzocyclohepten-2-ol
9) 3-methoxy-8-phenyl-9-propyl-6,7-dihydro-5H-benzocycloheptene
10) 6-phenyl-5-butyl-8,9-dihydro-7H-benzocyclohepten-2-ol
11) 9-butyl-3-methoxy-8-phenyl-6,7-dihydro-5H-benzocycloheptene
12) 6-(4-hydroxyphenyl)-5-methyl-8,9-dihydro-7H-benzocyclohepten-2-ol
13) 3-methoxy-8-(4-methoxyphenyl)-9-methyl-6,7-dihydro-5H-benzocycloheptene
14) 4-(2-methoxy-5-methyl-8,9-dihydro-7H-benzocyclohepten-6-yl)phenol
15) 6-(4-methoxyphenyl)-5-methyl-8,9-dihydro-7H-benzocyclohepten-2-ol
16) 6-(3-hydroxyphenyl)-5-methyl-8,9-dihydro-7H-benzocyclohepten-2-ol
17) 5-ethyl-6-(4-hydroxyphenyl)-8,9-dihydro-7H-benzocyclohepten-2-ol
18) 9-ethyl-3-methoxy-8-(4-methoxyphenyl)-6,7-dihydro-5H-benzocycloheptene
19) 4-(5-ethyl-2-methoxy-8,9-dihydro-7H-benzocyclohepten-6-yl)phenol
20) 5-ethyl-6-(4-methoxyphenyl)-8,9-dihydro-7H-benzocyclohepten-2-ol
21) 6-(4-hydroxyphenyl)-5-propyl-8,9-dihydro-7H-benzocyclohepten-2-ol
22) 3-methoxy-8-(4-methoxyphenyl)-9-propyl-6,7-dihydro-5H-benzocycloheptene
23) 4-(2-methoxy-5-propyl-8,9-dihydro-7H-benzocyclohepten-6-yl)phenol
24) 6-(4-methoxyphenyl)-5-propyl-8,9-dihydro-7H-benzocyclohepten-2-ol
25) 6-(4-hydroxyphenyl)-5-butyl-8,9-dihydro-7H-benzocyclohepten-2-ol
26) 6-(3-hydroxyphenyl)-5-ethyl-8,9-dihydro-7H-benzocyclohepten-2-ol
27) 6-(4-hydroxyphenyl)-5-trifluoromethyl-8,9-dihydro-7H-benzocyclohepten-2-ol
28) 6-(4-hydroxyphenyl)-5-pentafluoroethyl-8,9-dihydro-7H-benzocyclohepten-2-ol
29) 6-(4-hydroxyphenyl)-5-vinyl-8,9-dihydro-7H-benzocyclohepten-2-ol
30) 3-methoxy-8-(4-methoxyphenyl)-9-vinyl-6,7-dihydro-5H-benzocycloheptene
31) 6-(3-hydroxyphenyl)-5-vinyl-8,9-dihydro-7H-benzocyclohepten-2-ol
32) 6-(4-hydroxyphenyl)-6,7,8,9-tetrahydro-5H-benzocyclohepten-2-ol
33) 6-(3-hydroxyphenyl)-6,7,8,9-tetrahydro-5H-benzocyclohepten-2-ol
34) 6-(2-hydroxyphenyl)-6,7,8,9-tetrahydro-5H-benzocyclohepten-2-ol
35) 6-(4-hydroxyphenyl)-5-methyl-6,7,8,9-tetrahydro-5H-benzocyclohepten-2-ol
36) 6-(4-hydroxyphenyl)-5-ethyl-6,7,8,9-tetrahydro-5H-benzocyclohepten-2-ol
37) 6-(4-hydroxyphenyl)-6-methyl-6,7,8,9-tetrahydro-5H-benzocyclohepten-2-ol
38) 2-methoxy-6-(4-methoxy-phenyl)-6-methyl-6,7,8,9-tetrahydro-5H-benzocycloheptene
39) 4-(2-methoxy-6-methyl-6,7,8,9-tetrahydro-5H-benzocyclohepten-6-yl)phenol
40) 6-(4-methoxyphenyl)-6-methyl-6,7,8,9-tetrahydro-5H-benzocyclohepten-2-ol
41) 6-(4-hydroxyphenyl)-6-ethyl-6,7,8,9-tetrahydro-5H-benzocyclohepten-2-ol
42) 6-ethyl-2-methoxy-6-(4-methoxyphenyl)-6,7,8,9-tetrahydro-5H-benzocycloheptene
43) 4-(6-ethyl-2-methoxy-6,7,8,9-tetrahydro-5H-benzocyclohepten-6-yl)phenol
44) 6-ethyl-6-(4-methoxyphenyl)-6,7,8,9-tetrahydro-5H-benzocyclohepten-2-ol
45) 6-(4-hydroxyphenyl)-6-vinyl-6,7,8,9-tetrahydro-5H-benzocyclohepten-2-ol
46) 2-methoxy-6-(4-methoxyphenyl)-6-vinyl-6,7,8,9-tetrahydro-5H-benzocycloheptene
47) 4-(2-methoxy-6-vinyl-6,7,8,9-tetrahydro-5H-benzocyclohepten-6-yl)phenol
48) 6-(4-methoxyphenyl)-6-vinyl-6,7,8,9-tetrahydro-5H-benzocyclohepten-2-ol
49) 6-(4-hydroxyphenyl)-4-(2-trimethylsilanyl-ethyl)-6,7,8,9-tetrahydro-5H-benzocyclohepten-2-ol
50) 6-(4-hydroxyphenyl)-4-(2-methyldimethoxysilanylethyl)-6,7,8,9-tetrahydro-5H-benzocyclohepten-2-ol
51) 6-(4-hydroxyphenyl)-4-(2-triethoxysilanylethyl)-6,7,8,9-tetrahydro-5H-benzocyclohepten-2-ol
52) 6-(4-hydroxyphenyl)-4-vinyl-6,7,8,9-tetrahydro-5H-benzocyclohepten-2-ol
53) 3-hydroxy-8-(4-hydroxyphenyl)-6,7,8,9-tetrahydro-5H-benzocyclohepten-1-carbaldehyde
54) 3-hydroxy-8-(4-hydroxy-phenyl)-6,7,8,9-tetrahydro-5H-benzocyclohepten-1-carbonitrile
55) 6-(4-hydroxyphenyl)-4-ethyl-6,7,8,9-tetrahydro-5H-benzocyclohepten-2-ol
56) 6-(4-hydroxyphenyl)-4-(2-fluoroethyl)-6,7,8,9-tetrahydro-5H-benzocyclohepten-2-ol
57) 6-(4-hydroxyphenyl)-4-chloro-6,7,8,9-tetrahydro-5H-benzocyclohepten-2-ol
58) 6-(4-hydroxyphenyl)-5-methylene-6,7,8,9-tetrahydro-5H-benzocyclohepten-2-ol
59) 6-(4-hydroxyphenyl)-5,5-spirooxirane-6,7,8,9-tetrahydro-5H-benzocyclohepten-2-ol
60) 6-(4-hydroxyphenyl)-5,5-spirocyclopropyl-6,7,8,9-tetrahydro-5H-benzocyclohepten-2-ol
61) 5-ethyl-6-(4-hydroxyphenyl)-6-methyl-6,7,8,9-tetrahydro-5H-benzocyclohepten-2-ol
62) 5-ethyl-6-(4-hydroxyphenyl)-6-ethyl-6,7,8,9-tetrahydro-5H-benzocyclohepten-2-ol
63) 5-hydroxy-2-(2-hydroxy-8,9-dihydro-7H-benzocyclohepten-6-yl)benzonitrile
64) 6-(2-chloro-4-hydroxyphenyl)-8,9-dihydro-7H-benzocyclohepten-2-ol
65) 3-fluoro-6-(4-hydroxyphenyl)-8,9-dihydro-7H-benzocyclohepten-2-ol
66) 6-(3-fluoro-4-hydroxyphenyl)-8,9-dihydro-7H-benzocyclohepten-2-ol
67) 6-(2-fluoro-4-hydroxyphenyl)-8,9-dihydro-7H-benzocyclohepten-2-ol
68) 3-fluoro-4-(2-methoxy-8,9-dihydro-7H-benzocyclohepten-6-yl)-phenol
69) 6-(2,3-difluoro-4-hydroxyphenyl)-8,9-dihydro-7H-benzocyclohepten-2-ol
70) 6-(2-chloro-3-fluoro-4-hydroxyphenyl)-8,9-dihydro-7H-benzocyclohepten-2-ol
71) 6-(2,5-difluoro-4-hydroxyphenyl)-8,9-dihydro-7H-benzocyclohepten-2-ol
72) 6-(3,5-difluoro-4-hydroxyphenyl)-8,9-dihydro-7H-benzocyclohepten-2-ol
73) 6-(2,6-difluoro-4-hydroxyphenyl)-8,9-dihydro-7H-benzocyclohepten-2-ol
74) 6-(3-chloro-4-hydroxyphenyl)-8,9-dihydro-7H-benzocyclohepten-2-ol
75) 6-(2-methyl-4-hydroxyphenyl)-8,9-dihydro-7H-benzocyclohepten-2-ol
76) 6-(2-ethyl-4-hydroxyphenyl)-8,9-dihydro-7H-benzocyclohepten-2-ol
77) 6-(2-vinyl-4-hydroxyphenyl)-8,9-dihydro-7H-benzocyclohepten-2-ol
78) 6-(2-trifluoromethyl-4-hydroxyphenyl)-8,9-dihydro-7H-benzocyclohepten-2-ol
79) 6-(2-chloro-4-hydroxyphenyl)-5-methyl-8,9-dihydro-7H-benzocyclohepten-2-ol
80) 3-fluoro-6-(4-hydroxyphenyl)-5-methyl-8,9-dihydro-7H-benzocyclohepten-2-ol
81) 6-(3-fluoro-4-hydroxyphenyl)-5-methyl-8,9-dihydro-7H-benzocyclohepten-2-ol
82) 6-(2-fluoro-4-hydroxyphenyl)-5-methyl-8,9-dihydro-7H-benzocyclohepten-2-ol
83) 6-(2,3-difluoro-4-hydroxyphenyl)-5-methyl-8,9-dihydro-7H-benzocyclohepten-2-ol
84) 6-(2-chloro-3-fluoro-4-hydroxyphenyl)-5-methyl-8,9-dihydro-7H-benzocyclohepten-2-ol
85) 6-(2,5-difluoro-4-hydroxyphenyl)-5-methyl-8,9-dihydro-7H-benzocyclohepten-2-ol
86) 6-(3,5-difluoro-4-hydroxyphenyl)-5-methyl-8,9-dihydro-7H-benzocyclohepten-2-ol
87) 6-(2,6-difluoro-4-hydroxyphenyl)-5-methyl-8,9-dihydro-7H-benzocyclohepten-2-ol
88) 6-(3-chloro-4-hydroxyphenyl)-5-methyl-8,9-dihydro-7H-benzocyclohepten-2-ol
89) 6-(2-methyl-4-hydroxyphenyl)-5-methyl-8,9-dihydro-7H-benzocyclohepten-2-ol
90) 6-(2-ethyl-4-hydroxyphenyl)-5-methyl-8,9-dihydro-7H-benzocyclohepten-2-ol
91) 6-(2-vinyl-4-hydroxyphenyl)-5-methyl-8,9-dihydro-7H-benzocyclohepten-2-ol
92) 6-(2-trifluoromethyl-4-hydroxyphenyl)-5-methyl-8,9-dihydro-7H-benzocyclohepten-2-ol
93) 5-hydroxy-2-(2-hydroxy-5-methyl-8,9-dihydro-7H-benzocyclohepten-6-yl)-benzonitrile
94) 6-(2-chloro-4-hydroxyphenyl)-5-ethyl-8,9-dihydro-7H-benzocyclohepten-2-ol
95) 5-ethyl-3-fluoro-6-(4-hydroxyphenyl)-8,9-dihydro-7H-benzocyclohepten-2-ol
96) 5-ethyl-6-(3-fluoro-4-hydroxyphenyl)-8,9-dihydro-7H-benzocyclohepten-2-ol
97) 6-(2-fluoro-4-hydroxyphenyl)-5-ethyl-8,9-dihydro-7H-benzocyclohepten-2-ol
98) 6-(2,3-difluoro-4-hydroxyphenyl)-5-ethyl-8,9-dihydro-7H-benzocyclohepten-2-ol
99) 6-(2-chloro-3-fluoro-4-hydroxyphenyl)-5-ethyl-8,9-dihydro-7H-benzocyclohepten-2-ol
100) 6-(2,5-difluoro-4-hydroxyphenyl)-5-ethyl-8,9-dihydro-7H-benzocyclohepten-2-ol
101) 6-(3,5-difluoro-4-hydroxyphenyl)-5-ethyl-8,9-dihydro-7H-benzocyclohepten-2-ol
102) 6-(2,6-difluoro-4-hydroxyphenyl)-5-ethyl-8,9-dihydro-7H-benzocyclohepten-2-ol
103) 6-(3-chloro-4-hydroxyphenyl)-5-ethyl-8,9-dihydro-7H-benzocyclohepten-2-ol
104) 6-(2-methyl-4-hydroxyphenyl)-5-ethyl-8,9-dihydro-7H-benzocyclohepten-2-ol
105) 6-(2-ethyl-4-hydroxyphenyl)-5-ethyl-8,9-dihydro-7H-benzocyclohepten-2-ol
106) 6-(2-vinyl-4-hydroxyphenyl)-5-ethyl-8,9-dihydro-7H-benzocyclohepten-2-ol
107) 6-(2-trifluoromethyl-4-hydroxyphenyl)-5-ethyl-8,9-dihydro-7H-benzocyclohepten-2-ol
108) 5-hydroxy-2-(2-hydroxy-5-ethyl-8,9-dihydro-7H-benzocyclohepten-6-yl)-benzonitrile
109) 5-hydroxy-2-(2-hydroxy-5-propyl-8,9-dihydro-7H-benzocyclohepten-6-yl)-benzonitrile
110) 6-(2-chloro-4-hydroxyphenyl)-5-propyl-8,9-dihydro-7H-benzocyclohepten-2-ol
111) 3-fluoro-6-(4-hydroxyphenyl)-5-propyl-8,9-dihydro-7H-benzocyclohepten-2-ol
112) 6-(3-fluoro-4-hydroxyphenyl)-5-propyl-8,9-dihydro-7H-benzocyclohepten-2-ol
113) 6-(2-fluoro-4-hydroxyphenyl)-5-propyl-8,9-dihydro-7H-benzocyclohepten-2-ol
114) 6-(2,3-difluoro-4-hydroxyphenyl)-5-propyl-8,9-dihydro-7H-benzocyclohepten-2-ol
115) 6-(2-chloro-3-fluoro-4-hydroxyphenyl)-5-propyl-8,9-dihydro-7H-benzocyclohepten-2-ol
116) 6-(2,5-difluoro-4-hydroxyphenyl)-5-propyl-8,9-dihydro-7H-benzocyclohepten-2-ol
117) 6-(3,5-difluoro-4-hydroxyphenyl)-5-propyl-8,9-dihydro-7H-benzocyclohepten-2-ol
118) 6-(2,6-difluoro-4-hydroxyphenyl)-5-propyl-8,9-dihydro-7H-benzocyclohepten-2-ol
119) 6-(3-chloro-4-hydroxyphenyl)-5-propyl-8,9-dihydro-7H-benzocyclohepten-2-ol
120) 6-(4-hydroxy-2-methylphenyl)-5-propyl-8,9-dihydro-7H-benzocyclohepten-2-ol
121) 6-(2-ethyl-4-hydroxyphenyl)-5-propyl-8,9-dihydro-7H-benzocyclohepten-2-ol
122) 6-(4-hydroxy-2-vinylphenyl)-5-propyl-8,9-dihydro-7H-benzocyclohepten-2-ol
123) 6-(4-hydroxy-2-trifluoromethylphenyl)-5-propyl-8,9-dihydro-7H-benzocyclohepten-2-ol
124) 6-(2-chloro-4-hydroxyphenyl)-6-methyl-6,7,8,9-tetrahydro-5H-benzocyclohepten-2-ol
125) 3-fluoro-6-(4-hydroxyphenyl)-6-methyl-6,7,8,9-tetrahydro-5H-benzocyclohepten-2-ol
126) 6-(3-fluoro-4-hydroxyphenyl)-6-methyl-6,7,8,9-tetrahydro-5H-benzocyclohepten-2-ol
127) 6-(2-fluoro-4-hydroxyphenyl)-6-methyl-6,7,8,9-tetrahydro-5H-benzocyclohepten-2-ol
128) 6-(2,3-difluoro-4-hydroxyphenyl)-6-methyl-6,7,8,9-tetrahydro-5H-benzocyclohepten-2-ol
129) 6-(2-chloro-3-fluoro-4-hydroxyphenyl)-6-methyl-6,7,8,9-tetrahydro-5H-benzocyclohepten-2-ol
130) 6-(2,5-difluoro-4-hydroxyphenyl)-6-methyl-6,7,8,9-tetrahydro-5H-benzocyclohepten-2-ol
131) 6-(3,5-difluoro-4-hydroxyphenyl)-6-methyl-6,7,8,9-tetrahydro-5H-benzocyclohepten-2-ol
132) 6-(2,6-difluoro-4-hydroxyphenyl)-6-methyl-6,7,8,9-tetrahydro-5H-benzocyclohepten-2-ol
133) 6-(3-chloro-4-hydroxyphenyl)-6-methyl-6,7,8,9-tetrahydro-5H-benzocyclohepten-2-ol
134) 6-(2-methyl-4-hydroxyphenyl)-6-methyl-6,7,8,9-tetrahydro-5H-benzocyclohepten-2-ol
135) 6-(2-ethyl-4-hydroxyphenyl)-6-methyl-6,7,8,9-tetrahydro-5H-benzocyclohepten-2-ol
136) 6-(2-vinyl-4-hydroxyphenyl)-6-methyl-6,7,8,9-tetrahydro-5H-benzocyclohepten-2-ol
137) 6-(2-trifluoromethyl-4-hydroxyphenyl)-6-methyl-6,7,8,9-tetrahydro-5H-benzocyclohepten-2-ol
138) 5-hydroxy-2-(2-hydroxy-6-methyl-6,7,8,9-tetrahydro-5H-benzocyclohepten-6-yl)benzonitrile
139) 6-(2-chloro-4-hydroxyphenyl)-6-ethyl-6,7,8,9-tetrahydro-5H-benzocyclohepten-2-ol
140) 3-fluoro-6-(4-hydroxyphenyl)-6-ethyl-6,7,8,9-tetrahydro-5H-benzocyclohepten-2-ol
141) 6-(3-fluoro-4-hydroxyphenyl)-6-ethyl-6,7,8,9-tetrahydro-5H-benzocyclohepten-2-ol
142) 6-(2-fluoro-4-hydroxyphenyl)-6-ethyl-6,7,8,9-tetrahydro-5H-benzocyclohepten-2-ol
143) 6-(2,3-difluoro-4-hydroxyphenyl)-6-ethyl-6,7,8,9-tetrahydro-5H-benzocyclohepten-2-ol
144) 6-(2-chloro-3-fluoro-4-hydroxyphenyl)-6-ethyl-6,7,8,9-tetrahydro-5H-benzocyclohepten-2-ol
145) 6-(2,5-difluoro-4-hydroxyphenyl)-6-ethyl-6,7,8,9-tetrahydro-5H-benzocyclohepten-2-ol
146) 6-(3,5-difluoro-4-hydroxyphenyl)-6-ethyl-6,7,8,9-tetrahydro-5H-benzocyclohepten-2-ol
147) 6-(2,6-difluoro-4-hydroxyphenyl)-6-ethyl-6,7,8,9-tetrahydro-5H-benzocyclohepten-2-ol
148) 6-(3-chloro-4-hydroxyphenyl)-6-ethyl-6,7,8,9-tetrahydro-5H-benzocyclohepten-2-ol
149) 6-(2-methyl-4-hydroxyphenyl)-6-ethyl-6,7,8,9-tetrahydro-5H-benzocyclohepten-2-ol
150) 6-(2-ethyl-4-hydroxyphenyl)-6-ethyl-6,7,8,9-tetrahydro-5H-benzocyclohepten-2-ol
151) 6-(2-vinyl-4-hydroxyphenyl)-6-ethyl-6,7,8,9-tetrahydro-5H-benzocyclohepten-2-ol
152) 6-(2-trifluoromethyl-4-hydroxyphenyl)-6-ethyl-6,7,8,9-tetrahydro-5H-benzocyclohepten-2-ol
153) 5-hydroxy-2-(2-hydroxy-6-ethyl-6,7,8,9-tetrahydro-5H-benzocyclohepten-6-yl)benzonitrile
154) 6-(2-chloro-4-hydroxyphenyl)-6,7,8,9-tetrahydro-5H-benzocyclohepten-2-ol
155) 3-fluoro-6-(4-hydroxyphenyl)-6,7,8,9-tetrahydro-5H-benzocyclohepten-2-ol
156) 6-(3-fluoro-4-hydroxyphenyl)-6,7,8,9-tetrahydro-5H-benzocyclohepten-2-ol
157) 6-(2-fluoro-4-hydroxyphenyl)-6,7,8,9-tetrahydro-5H-benzocyclohepten-2-ol
158) 6-(2,3-difluoro-4-hydroxyphenyl)-6,7,8,9-tetrahydro-5H-benzocyclohepten-2-ol
159) 6-(2-chloro-3-fluoro-4-hydroxyphenyl)-6,7,8,9-tetrahydro-5H-benzocyclohepten-2-ol
160) 6-(2,5-difluoro-4-hydroxyphenyl)-6,7,8,9-tetrahydro-5H-benzocyclohepten-2-ol
161) 6-(3,5-difluoro-4-hydroxyphenyl)-6,7,8,9-tetrahydro-5H-benzocyclohepten-2-ol
162) 6-(2,6-difluoro-4-hydroxyphenyl)-6,7,8,9-tetrahydro-5H-benzocyclohepten-2-ol
163) 6-(3-chloro-4-hydroxyphenyl)-6,7,8,9-tetrahydro-5H-benzocyclohepten-2-ol
164) 6-(2-methyl-4-hydroxyphenyl)-6,7,8,9-tetrahydro-5H-benzocyclohepten-2-ol
165) 6-(2-ethyl-4-hydroxyphenyl)-6,7,8,9-tetrahydro-5H-benzocyclohepten-2-ol
166) 6-(2-vinyl-4-hydroxyphenyl)-6,7,8,9-tetrahydro-5H-benzocyclohepten-2-ol
167) 6-(2-trifluoromethyl-4-hydroxy-phenyl)-6,7,8,9-tetrahydro-5H-benzocyclohepten-2-ol
168) 5-hydroxy-2-(2-hydroxy-6,7,8,9-tetrahydro-5H-benzocyclohepten-6-yl)-benzonitrile
169) 5-allyl-6-(4-hydroxyphenyl)-8,9-dihydro-7H-benzocyclohepten-2-ol
170) 9-allyl-3-methoxy-8-(4-methoxyphenyl)-6,7-dihydro-5H-benzocycloheptene
171) 6-allyl-2-methoxy-6-(4-methoxyphenyl)-6,7,8,9-tetrahydrobenzocyclohepten-5-one
172) 6-allyl-2-methoxy-6-(4-methoxyphenyl)-6,7,8,9-tetrahydro-5H-benzocycloheptene
173) 6-allyl-6-(4-hydroxyphenyl)-6,7,8,9-tetrahydro-5H-benzocyclohepten-2-ol
174) 2-methoxy-6-(4-methoxyphenyl)-6,7,8,9-tetrahydrobenzocyclohepten-5-on 175) 2-methoxy-6-(4-methoxyphenyl)-6-propyl-6,7,8,9-tetrahydro-benzocyclohepten-5-one
176) 2-methoxy-6-(4-methoxyphenyl)-6-propyl-6,7,8,9-tetrahydro-5H-benzocycloheptene
177) 6-(4-hydroxyphenyl)-6-propyl-6,7,8,9-tetrahydro-5H-benzocyclohepten-2-ol
178) 5-allyl-6-(4-hydroxyphenyl)-6-methyl-6,7,8,9-tetrahydro-5H-benzocyclohepten-2-ol
179) 5-allyl-6-(4-hydroxyphenyl)-6-ethyl-6,7,8,9-tetrahydro-5H-benzocyclohepten-2-ol
180) 6-(4-hydroxyphenyl)-4-trifluoromethyl-6,7,8,9-tetrahydro-5H-benzocyclohepten-2-ol
181) 4,6-diethyl-6-(4-hydroxyphenyl)-6,7,8,9-tetrahydro-5H-benzocyclohepten-2-ol
182) 4-ethyl-6-(4-hydroxyphenyl)-6-methyl-6,7,8,9-tetrahydro-5H-benzocyclohepten-2-ol
183) 6-(3-hydroxyphenyl)-6-methyl-6,7,8,9-tetrahydro-5H-benzocyclohepten-2-ol
184) 6-(3-hydroxyphenyl)-5-butyl-6,7,8,9-tetrahydro-5H-benzocyclohepten-2-ol
185) 6-(4-hydroxyphenyl)-5-propyl-6,7,8,9-tetrahydro-5H-benzocyclohepten-2-ol
186) 6-(3-hydroxyphenyl)-5-propyl-6,7,8,9-tetrahydro-5H-benzocyclohepten-2-ol
187) 6-(4-hydroxyphenyl)-5-propenyl-8,9-dihydro-7H-benzocyclohepten-2-ol
188) 6-(4-hydroxyphenyl)-5,5-dimethyl-8,9-dihydro-5H-benzocyclohepten-2-ol
189) 6-(4-hydroxyphenyl)-5,5-dimethyl-6,7,8,9-tetrahydro-5H-benzocyclohepten-2-ol
190) 5-ethyl-6-(4-hydroxyphenyl)-5-methyl-8,9-dihydro-5H-benzocyclohepten-2-ol
191) 5,5-diethyl-6-(4-hydroxyphenyl)-8,9-dihydro-5H-benzocyclohepten-2-ol
192) 6-(4-hydroxyphenyl)-5-phenyl-8,9-dihydro-7H-benzocyclohepten-2-ol
193) 6-(4-hydroxyphenyl)-5-(3-methylthiophen-2-yl)-8,9-dihydro-7H-benzocyclohepten-2-ol

### Preparation of the Compounds According to the Invention

Benzocycloheptene derivatives according to the present invention can be prepared by the following synthetic route:

2-Methoxy-5,7,8,9-tetrahydrobenzocyclohepten-6-one is obtained by known methods from 6-methoxytetralone by olefination for example with methyltriphenylphosphonium bromide/base such as, for example, KOt-Bu and rearrangement with TI(NO₃)₃ (TTN), [hydroxy(tosyloxy)iodo]benzene HTIB or AgNO₃/I₂. Mono- or dialkylation of the 6-methoxytetralone before the Wittig reaction and the TTN rearrangement lead to the corresponding 7-substituted 2-methoxybenzocycloheptan-6-ones.

Use of higher alkyltriphenylphosphonium bromides in the Wittig reaction leads after TTN rearrangement to corresponding 5-substituted 2-methoxybenzocycloheptan-6-ones. [E. C. Taylor et al., Tetrahed. Letters, 1977, 1827-1830; A. I. Khalaf et al., J. Chem. Soc. Perkin Trans. 1, 12, 1992, 1475-1482; C. M. M. da Conceicao et al., J. Chem. Res. Miniprint, 9, 1995, 2161-2194.]

The unsubstituted and 5- or 7-substituted 2-methoxy-5,7,8,9-tetrahydrobenzocyclohepten-6-ones can then be prepared by further alkylation using alkyl halides and a base such as, for example, KOt-Bu the corresponding more highly substituted 2-methoxy-5,7,8,9-tetrahydrobenzocyclohepten-6-ones.

The dialkylation of unsubstituted 2-methoxy-5,7,8,9-tetrahydrobenzocyclohepten-6-ones using alkyl dihalides leads to spiro compounds.

The unsubstituted or alkyl-substituted 2-methoxy-5,7,8,9-tetrahydrobenzocyclohepten-6-ones are converted into an enol compound such as, for example, enol triflate or enol nonaflate. Subsequent palladium-catalysed coupling (e.g. Suzuki coupling) with substituted boronic acids leads to the formation of the compounds according to the invention, the benzocycloheptenes according to formula I. The compounds are converted into the corresponding benzocycloheptan-5-ones by hydroboration and subsequent oxidation.

The prepared benzocyclohepten-5-ones can be converted by known reactions such as, for example:
reductions with hydrides (e.g. borohydrides, boranates, alanates) or with hydrogen with metal catalysis (e.g. Pd, Pt catalysis)
dehydration (e.g. acid-catalysed)
6-alkylation, 5-alkylation (e.g. with alkyl halides)
deoxygenation (e.g. with silanes)
enolization in position 5 (e.g. as triflate or nonaflate) and palladium coupling (e.g. Stille or Suzuki coupling)
ruthenium-catalysed 4-alkylation
olefination in position 5 (e.g. Wittig olefination)
ether cleavage (e.g. boron halides for methyl ethers)
   into the compounds according to the invention of the formula I, it being possible for the introduced substituents or groups to be converted further by chemical reactions known to the skilled person.

Definition of the substituents in the compounds of the formula I:
“(C₁-C₄)Alkyl radical” means in the context of the present invention a branched or straight-chain alkyl radical having 1 to 4 carbon atoms which may be substituted by F, Cl, Br, hydroxy groups or (C₁-C₄)alkyl-O radical (OMe) or CN. Exemplary embodiments which may be mentioned are a methyl, ethyl, n-propyl, i-propyl, n-butyl, i-butyl, tert-butyl, 1-bromoethyl, 2-bromoethyl, 1-chloroethyl, 2-chloroethyl, 1-fluoroethyl, 2-fluoroethyl radical. In the context of the present invention, methyl, ethyl, propyl and isopropyl are preferred for the (C₁-C₄)-alkyl radical.
“(C₃-C₆)Cycloalkyl radical” means in the context of the present invention a carbocyclic radical having 3 to 6 carbon atoms which may be substituted by F, Cl, Br, hydroxy groups or (C₁-C₄)alkyl-O radical (OMe) or CN. Exemplary embodiments which may be mentioned are the cyclopropyl, cyclopentyl and the cyclohexyl radical. In the context of the present invention, the cyclopentyl radical is preferred for the (C₃-C₆)cycloalkyl radical.
“(C₁-C₁₄)Acyl radical” means in the context of the present invention an aliphatic, straight- or branched-chain, saturated or unsaturated (C₁-C₁₄)-carbonyl radical, an aromatic carbonyl radical or the carbonyl radical derived from an a- or β-amino acid, which may be substituted by F, Cl or Br. Suitable radicals of this type are for example the carbonyl radicals derived from the following carboxylic acids: formic acid, acetic acid, propionic acid, butyric acid, isobutyric acid, valeric acid, isovaleric acid, pivalic acid, lauric acid, myristic acid, acrylic acid, propiolic acid, methacrylic acid, crotonic acid, isocrotonic acid, oleic acid, elaidic acid. Dicarboxylic acids: oxalic acid, malonic acid, succinic acid, glutaric acid, adipic acid, pimelic acid, suberic acid, azelaic acid, sebacic acid, maleic acid, fumaric acid, muconic acid, citraconic acid, mesaconic acid, benzoic acid, phthalic acid, isophthalic acid, terephthalic acid, naphthoic acid, o-, m- and p-toluic acids, hydratropic acid, atropic acid, cinnamic acid, nicotinic acid, isonicotinic acid, alanine, β-alanine, arginine, cysteine, cystine, glycine, histidine, leucine, isoleucine, phenylalanine, proline.
“(C₁-C₄)-Alkenyl radical” means in the context of the present invention a branched or straight-chain alkenyl radical having 1 to 4 carbon atoms which may be substituted by F, Cl, Br, hydroxy groups or (C₁-C₄)alkyl-O radical (OMe) or CN. Exemplary embodiments of the present invention which may be mentioned are a vinyl, prop-1-enyl, allyl, 2-methylallyl, i-propenyl, n-but-1-enyl, n-but-2-enyl, 2-methylpropenyl, 2-bromovinyl, 3-bromopropenyl, 2-bromopropenyl, 1-bromoallyl radical, 3-bromoallyl, 3-chloroallyl, 3-chloropropenyl, 3-fluoroallyl or 3-fluoropropenyl radical.
“Perfluoro(C₁-C₄)-alkyl radical” means in the context of the present invention a branched or straight-chain perfluorinated alkyl radical having 1 to 4 carbon atoms. Exemplary embodiments of the present invention which may be mentioned are a trifluoromethyl, pentafluoroethyl, heptafluoro-n-propyl or heptafluoro-isopropyl radical.
“(C₂-C₄)Alkanediyl radical” or “(C₁-C₄)alkanediyl radical” means in the context of the present invention a branched or straight-chain radical having 1 to 4 or 2 to 4 carbon atoms. Exemplary embodiment which may be mentioned is —CH₂—CH₂—CH₂—CH₂.
“(C₂-C₆)Alkenediyl radical” means in the context of the present invention a branched or straight-chain radical having 2 to 6 carbon atoms. Exemplary embodiments which may be mentioned are —CH₂—CH═CH—CH₂—, CH₂—CH═CH—CH₂—CH₂—, —CH₂—CH₂—CH═CH—CH₂—CH₂—.
“Perfluoro(C₁-C₄)-alkenyl radical” means in the context of the present invention a branched or straight-chain perfluorinated alkenyl radical having 1 to 4 carbon atoms which may be substituted by chlorine or bromine. Exemplary embodiments of the present invention which may be mentioned are a trifluorovinyl, pentafluoropropenyl, pentafluoroallyl, 2-chloro-1,2-difluorovinyl, heptafluorobut-2-enyl or heptafluorobut-3-enyl radical.
Aryl radical means a phenyl radical which may be substituted by F, Cl, OMe, Me, Et, OEt, CF₃. Examples which may be mentioned are phenyl, 2-fluorophenyl, 3-fluorophenyl, 2-chlorophenyl, 3-chlorophenyl, toluenyl.
Heteroaryl radical means a thiophene, pyridine, pyrrolidine and furan radical which may be substituted by F, Cl, OMe, Me, Et, OEt, CF₃. Exemplary embodiments of the present invention which may be mentioned are thiophen-2-yl, thiophen-3-yl, pyridin-2-yl, pyridin-3-yl, pyridin-4-yl, N-methylpyrrolidin-2-yl, 3-methylthiophen-2-yl and 3-chlorothiophen-2-yl.
Both the aryl and the heteroaryl radical may be substituted.

Substituents which may be mentioned for an aryl or heteroaryl radical are for example a methyl-, ethyl-, trifluoromethyl-, pentafluoroethyl-, trifluoromethylthio-, methoxy-, ethoxy-, nitro-, cyano-, halogen- (fluorine, chlorine, bromine, iodine), hydroxy-, amino-, mono((C₁-C₄)alkyl)- or di((C₁-C₄)alkyl)amino, where the two alkyl groups are identical or different, di(aralkyl)amino, where the two aralkyl groups are identical or different.
The term “halogen atom” means in the context of the present invention a fluorine, chlorine, bromine or iodine atom.
(C₁-C₄)Alkynyl radical means in the context of the present invention an alkynyl radical having 1 to 4 carbon atoms which may be substituted by fluorine, chlorine or bromine. Exemplary embodiments of the present invention which may be mentioned are an ethynyl, prop-1-ynyl, prop-2-ynyl, but-2-ynyl or trifluoroprop-1-ynyl radical.
(C₁-C₄)Alkyl-O radical means in the context of the present invention a (C₁-C₄)alkyl radical which is linked via oxygen and has 1 to 4 carbon atoms and which may be substituted by fluorine, chlorine and bromine. Exemplary embodiments of the present invention which may be mentioned are a methoxy, ethoxy or 2-fluoroethoxy radical.
“(C₃-C₆)Cycloalkyl-O radical” means in the context of the present invention a (C₃-C₆)cycloalkyl radical which is linked via an oxygen and which may be substituted by F, Cl, Br, hydroxy groups or (C₁-C₄)alkyl-O radical (OMe) or CN. Exemplary embodiments which may be mentioned are the cyclopropyloxy, cyclopentyloxy and a cyclohexyloxy radical. In the context of the present invention, the cyclopentyloxy radical is preferred for the (C₃-C₆)cycloalkyl-O radical.
(C₁-C₁₄)Acyl-O radical mentioned in the context of the present invention is a radical derived from esterification of a free hydroxyl group with an aliphatic, straight- or branched-chain, saturated or unsaturated (C₁-C₁₄)mono- or polycarboxylic acid or an aromatic carboxylic acid or with an α- or β-amino acid. Suitable radicals are for example the carbonyl radicals derived from the following carboxylic acids: formic acid, acetic acid, propionic acid, butyric acid, isobutyric acid, valeric acid, isovaleric acid, pivalic acid, lauric acid, myristic acid, acrylic acid, propiolic acid, methacrylic acid, crotonic acid, isocrotonic acid, oleic acid, elaidic acid. Dicarboxylic acids: oxalic acid, malonic acid, succinic acid, glutaric acid, adipic acid, pimelic acid, suberic acid, azelaic acid, sebacic acid, maleic acid, fumaric acid, muconic acid, citraconic acid, mesaconic acid, benzoic acid, phthalic acid, isophthalic acid, terephthalic acid, naphthoic acid, o-, m- and p-toluic acids, hydratropic acid, atropic acid, cinnamic acid, nicotinic acid, isonicotinic acid, alanine, β-alanine, arginine, cysteine, cystine, glycine, histidine, leucine, isoleucine, phenylalanine, proline.
“Silyl-” mentioned in the context of the present invention is a Si(R)₃ where R is an H atom or a (C₁-C₄)-alkyl-.

Free hydroxyl groups in the compounds of the general formula I may be esterified with an aliphatic, straight- or branched-chain, saturated or unsaturated (C₁-C₁₄)mono- or polycarboxylic acid or an aromatic carboxylic acid or with an a- or β-amino acid. Examples of carboxylic acids of this type suitable for esterification are:

monocarboxylic acids, formic acid, acetic acid, propionic acid, butyric acid, isobutyric acid, valeric acid, isovaleric acid, pivalic acid, lauric acid, myristic acid, acrylic acid, propiolic acid, methacrylic acid, crotonic acid, isocrotonic acid, oleic acid, elaidic acid. Dicarboxylic acids: oxalic acid, malonic acid, succinic acid, glutaric acid, adipic acid, pimelic acid, suberic acid, azelaic acid, sebacic acid, maleic acid, fumaric acid, muconic acid, citraconic acid, mesaconic acid.

Aromatic carboxylic acids: benzoic acid, phthalic acid, isophthalic acid, terephthalic acid, naphthoic acid, o-, m- and p-toluic acid, hydratropic acid, atropic acid, cinnamic acid, nicotinic acid, isonicotinic acid.

Suitable amino acids are the representatives of this class of substances which are well known to the skilled person, for example alanine, β-alanine, arginine, cysteine, cystine, glycine, histidine, leucine, isoleucine, phenylalanine, proline etc.

The esters according to the invention of the compounds according to the present invention exhibit advantages as prodrugs over the unesterified active ingredients in relation to their mode of administration, their type of effect, potency and duration of effect.

Pharmacokinetic and pharmacodynamic advantages are also shown by the sulphamate derivatives according to the invention. Effects of this type have already been described for sulphamates derived from estrogens with a natural absolute configuration (J. Steroid Biochem. Molec. Biol, 55, 395-403 (1995); Exp. Opinion Invest. Drugs 7, 575-589 (1998)).

The substituents R^{5x}, R^{5y}, R⁶, R^{7x}, R^{7y} may in each case be in the a or β position.

The novel estrogens described in the present patent can be employed as single component in pharmaceutical preparations or in combination in particular with progestogens, androgens or anti-estrogens.

The substances and the pharmaceutical products containing them are suitable for example as components of oral contraceptives, for instance in combination with a progestogen.

The present patent application describes benzocycloheptene derivatives for the treatment of estrogen deficiency-related diseases and conditions.

The invention also relates to pharmaceutical products which comprise at least one compound of the general formula I (or physiologically tolerated addition salts with organic and inorganic acids thereof) and to the use of these compounds for the manufacture of medicaments, in particular for the following indications.

The compounds can be employed both after oral and after parenteral administration for the following indications.

The novel non-steroidal estrogens described in the present patent application can be employed as single component in pharmaceutical preparations or in combination in particular with anti-estrogens or progestogens. Combination of the selective estrogens with ERα-selective anti-estrogens, or with anti-estrogens which have selective peripheral activity, i.e. do not cross the blood-brain barrier, is particularly preferred.

The substances and the pharmaceutical products comprising them are particularly suitable for the treatment of peri- and post-menopausal symptoms, especially hot flushes, sleep disorders, irritability, mood fluctuations, incontinence, vaginal atrophy, hormone deficiency-related affective disorders. The substances are likewise suitable for hormone replacement and the therapy of hormone deficiency-related symptoms associated with ovarian dysfunction related to surgery, medication or other factors.

The compounds are also suitable for alleviating the symptoms of the andropause and menopause, i.e. for male and female hormone replacement therapy (HRT), in particular both for prevention and for treatment, also for the treatment of the symptoms associated with dysmenorrhoea, and for the treatment of acne.

The substances additionally exert an estrogen-like effect on the vascular system and brain functions.

The substances can further be employed for the prevention of cardiovascular disorders, especially vascular disorders such as atherosclerosis, for inhibiting the proliferation of arterial smooth muscle cells, for treating primary pulmonary hypertension and for preventing hormone deficiency-related neurodegenerative disorders such as Alzheimer's disease, and hormone deficiency-related impairment of memory and learning ability.

The substances can further be employed for the treatment of inflammatory disorders and disorders of the immune system, in particular autoimmune diseases such as, for example, rheumatoid arthritis and MS. The substances according to the invention can be employed in particular for preventing and treating endometriosis.

The non-steriodal estrogens described in the present patent application are additionally suitable for the prevention and treatment of alopecia.

The described substances are suitable for the therapy of hot flushes, for improving ovarian function and stimulating folliculogenesis and as component of novel contraceptives.

The compounds may additionally be used for the treatment of male fertility impairments and prostatic disorders.

The substances are additionally suitable for the prevention and therapy of estrogen-dependent gastrointestinal carcinomas.

Finally, the compounds of the general formula I can be used in conjunction with progesterone receptor antagonists, in particular for use in hormone replacement therapy and for the treatment of gynaecological disorders.

A therapeutic product comprising an estrogen and a pure anti-estrogen for simultaneous, sequential or separate use for selective estrogen therapy of peri- or postmenopausal conditions has been described in EP-A 0 346 014.

The amount to be administered of a compound of the general formula I varies within a wide range and can cover every effective amount. Depending on the condition to be treated and the mode of administration, the amount of the administered compound can be 0.01 μg/kg-10 mg/kg of body weight, preferably 0.04 μg/kg-1 mg/kg of body weight, per day.

In humans, this corresponds to a dose of from 0.8 μg to 800 mg, preferably 3.2 μg to 80 mg, a day.

A dose unit comprises according to the invention from 1.6 μg to 200 mg of one or more compounds of the general formula I.

The compounds according to the invention and the acid addition salts are suitable for the production of pharmaceutical compositions and preparations. The pharmaceutical compositions or medicaments contain as active ingredient one or more of the compounds according to the invention or the acid addition salts thereof, where appropriate mixed with other pharmacologically or pharmaceutically active substances. The medicaments are produced in the known manner, it being possible to use the known and conventional pharmaceutical excipients and other conventional carriers and diluents.

Examples of suitable carriers and excipients of these types are those recommended or indicated in the following references as excipients for pharmacy, cosmetics and adjoining areas: Ullmans Encyklopädie der technischen Chemie, volume 4 (1953), pages 1 to 39; Journal of Pharmaceutical Sciences, volume 52 (1963), pages 918 et seq., H. v. Czetsch-Lindenwald, Hilfsstoffe für Pharmazie and angrenzende Gebiete; Pharm. Ind., No. 2, 1961, page 72 et seq.: Dr. H. P. Fiedler, Lexikon der Hilfsstoffe für Pharmazie, Kosmetik and angrenzende Gebiete, Cantor K G. Aulendorf in Württemberg 1971.

The compounds can be administered orally or parenterally, for example intraperitoneally, intramuscularly, subcutaneously or percutaneously. The compounds can also be implanted into tissue.

Capsules, pills, tablets, coated tablets etc. are suitable for oral administration. The dosage units may, besides the active ingredient, comprise a pharmaceutically acceptable carrier such as, for example, starch, sugar, sorbitol, gelatine, lubricant, silica, talc etc.

For parenteral administration, the active ingredients can be dissolved or suspended in a physiologically tolerated diluent. Diluents very frequently used are oils with or without addition of a solubilizer, of a surface-active agent, of a suspending agent or emulsifier. Examples of oils which are used are olive oil, peanut oil, cottonseed oil, soya oil, castor oil and sesame oil.

The compounds can also be used in the form of a depot injection or of an implant product, each of which may be formulated so that delayed release of active ingredient is made possible.

Implants may comprise as inert materials for example biodegradable polymers or synthetic silicones such as, for example, silicone rubber.

The active ingredients may additionally be incorporated for example in a patch for percutaneous administration.

Various polymers such as, for example, silicone polymers, ethylene-vinyl acetate, polyethylene or polypropylene are suitable for producing intravaginal systems (e.g. vaginal rings) or intrauterine systems (e.g. pessaries, coils, IUSs, Mirena®) loaded with active compounds of the general formula I for local administration. In order to improve the bioavailability of the active ingredient, the compounds can also be formulated as cyclodextrin clathrates. For this purpose, the compounds are reacted with a-, β- or γ-cyclodextrin or derivatives thereof.

The compounds of the general formula I can also be encapsulated according to the invention with liposomes.

### Methods

### Estrogen Receptor-Binding Studies

The binding affinity of the novel estrogens was assayed in competitive experiments using 3H-oestradiol as ligand on estrogen receptor preparations from rat prostate and rat uterus. The preparation of the prostate cytosol and the estrogen receptor assay with the prostate cyclosol was carried out as described by Testas et al. (1981) (Testas J. et al., 1981, Endocrinology 109: 1287-1289).

The preparation of rat uterine cytosol, and the receptor assay with the ER-containing cytosol were carried in principle as described by Stack and Gorski, 1985, (Stack, Gorski 1985, Endocrinology 117, 2024-2032) with some modifications as described in Fuhrmann et al. (1995) (Fuhrmann U. et al. 1995, Contraception 51: 45-52).

It is assumed in this connection that ERβ predominates over ERa in the rat prostate, and ERα predominates over ERβ in the rat uterus. Table 1 shows that the ratio of the binding to prostate receptor and uterus receptor agrees qualitatively with the quotient of the relative binding affinity (RBA) on human ERβ and ERα from rat (according to Kuiper et al. (1996), Endocrinology 138: 863-870) (Table 1).

In addition, the predictivity of the ‘prostate ER versus uterus ER assay system’ in relation to a tissue-selective effect was confirmed by in vivo investigations. Substances with preference for prostate ER are dissociated in relation to the effect on uterus in favour of the effect on ovary in vivo.

Table 2 shows the results for the compounds to be used according to the invention.

### Cellular In Vitro Assay to Determine the Estrogen Receptor-a and -β Activity

### Abbreviations:

DMEM Dulbecco's modified Eagle medium
DNA deoxynucleic acid
FCS fetal calf serum
HEPES 4-(2-hydroxyethyl)-1-piperazineethanesulphonic acid
PCR polymerase chain reaction

Modulators of the human estrogen receptors-a and -β (ERα and ERβ) are identified, and the activity of the substances described herein is quantified, with the aid of recombinant cell lines. These cells are originally derived from a hamster ovary epithelial cell (Chinese Hamster Ovary, CHO K1, ATCC: American Type Culture Collection, VA 20108, USA).

An established chimera system in which the ligand-binding domains of human steroid hormone receptors are fused to the DNA-binding domain of the yeast transcription factor GAL4 are used in this CHO K1 cell line. The GAL4-steroid hormone receptor chimeras produced in this way are cotransfected and stably expressed with a reporter construct in the CHO cells.

### Clonings:

To generate the GAL4-steroid hormone receptor chimeras, the GAL4 DNA-binding domain (amino acids 1-147) from the vector pFC2-dbd (from stratagene) is cloned with the PCR-amplified ligand-binding domains of the estrogen recpetor a (ERa, Genbank accession number NM00125, amino acids 282-595) and of the estrogen receptor β (ERβ, Genbank accession number AB006590, amino acids 223-530) into the vector pIRES2 (from Clontech). The reporter construct, which comprises five copies of the GAL4 binding site upstream of a thymidine kinase promoter, leads to expression of firefly luciferase (Photinus pyralis) after activation and binding of the GAL4-estrogen receptor chimeras by specific agonists.

Assay procedure: the stock cultures of ERa and ERβ cells are routinely cultured in DMEM/F12 medium, 10% FCS, 1% Hepes, 1% penicillin/streptomycin, 1 mg/ml G418, and 5 μg/ml puromycin. On the day before the assay, the ERa and ERβ cells are plated out in Opti-MEM medium (Optimem, from Invitrogen, 2.5% activated carbon-purified FCS from Hyclone, 1% Hepes) in 96- (or 384) well microtitre plates and kept in a cell incubator (96% humidity, 5% v/v CO₂, 37° C.). On the day of the assay, the substances to be tested are taken up in the abovementioned medium and added to the cells. If it is intended to investigate possible antagonistic properties of test substances, the estrogen receptor agonist 17-β oestradiol (from Sigma) is added 10 to 30 minutes after addition of the test substances, but no additional addition of 17-β oestradiol takes place in the investigation of agonistic properties. After a further incubation time of 5 to 6 hours, the cells are lysed with a luciferin/Triton buffer, and the luciferase activity is measured with the aid of a video camera. The measured relative light units as a function of the substance concentration result in a sigmoidal stimulation curve. The EC₅₀ and IC₅₀ values are calculated with the aid of the GraphPad PRISM (version 3.02) computer program.

Table 3 shows the results for the compounds to be used according to the invention.

### Investigations of the Dissociation of the Effects on Uterus and Pituitary

The effect on ERbeta is detected in vivo through a stimulation of folliculogenesis in the female rat ovary. The effect of ERalpha is measured by an increase in the weight of the uterus in ovarectomized rats.

The investigations concerning the effect on uterine growth and ovulation (indirect effect through an influence on the secretion of pituitary hormones) are carried out in adult female rats (body weight 220-250 g). The substances are administered subcutaneously once a day for 4 days. The first administration takes place in the metoestrus stage of the cycle. Autopsy takes place one day after the last administration. The number of oocytes in each fallopian tube (effect on ovulation) is determined and analysed, as is the wet weight of the uterus.

Whereas the effect of oestradiol is a dose-dependent inhibition of ovulation and an increase in the weight of the uterus with an ED₅₀ of 0.004 mg/kg of body weight, the investigated substance shows no effect on the pituitary and the uterus.

### Investigations of the Effect on the Ovary:

The substances are tested in vivo on hypophysectomized rats. In a modification of this surgical method, a GnRH antagonist (cetrorelix) is instead administered to the animals. Possible stimulation of follicle growth (maturation) in the ovary by the substance is investigated. The ovary weight is determined and analysed. Five animals (body weight 40-50 g, age 3 weeks) are employed randomly in each treatment group. The animals receive slightly acidified tap water and a standard diet ad libitum and are kept in Makrolon cages in air-conditioned rooms with controlled illumination (light for 12 h, dark for 12 h).

For subcutaneous administration, the test substances and the reference substance (E2, oestradiol) are dissolved in benzyl benzoate/castor oil (1+4, v/v).

Juvenile female rats are hypophysectomized on day 0 and treated 1× a day for 4 days subcutaneously either with the reference substance oestradiol, the test substance or the vehicle solution (benzyl benzoate/castor oil). In the modified method, 0.5 mg/animal/day cetrorelix is administered together with the test substances or oestradiol or vehicle solution for 4 days. 24 hours after the last administration, the animals are sacrificed and both the ovary weight and, after histological workup, the different follicle types are determined. The test substance shows distinct stimulation of the ovary weight and an increase in the number of preantral follicles (profertile effect).

The test substance shows a distinct dissociation between the effect on the ovary itself and the effect on the uterus and pituitary and is thus, because of its profertile, follicle-stimulating effect, explicitly suitable for the treatment of female infertility.

### Preparation of the Compounds According to the Invention

The compounds according to the invention can be prepared by the synthetic route described above.

The carboxylic esters according to the invention are prepared in analogy to the esters derived from natural steroid active substances (see, for example, Pharmazeutische Wirkstoffe, Synthesen, Patente, Anwendungen; A. Kleernann, J. Engel, Georg Thieme Verlag Stuttgart 1978. Arzneimittel, Fortschritte 1972 to 1985; A. Kleemann, E. Lindner, J. Engel (editors), VCH 1987, pp. 773-814).

The compounds according to the invention of the general formula I are prepared as described in the examples. Further compounds of the general formula I can be obtained by an analogous procedure using homologous reagents to the reagents described in the examples.

Etherification and/or esterification of free hydroxy groups takes place by methods familiar to the skilled person.

### EXAMPLES

### 1. Preparation of the Nonaflates

### 1.1. 2-Methoxy-8,9-dihydro-7H-benzocyclohepten-6-yl nonafluorobutane-1-sulphonate

250 mg of 2-methoxy-5,7,8,9-tetrahydrobenzocyclohepten-6-one are dissolved in 4 ml of toluene at 0° C. Addition of 0.7 ml of DBU is followed by addition of 0.78 ml of nonaflyl fluoride. The reaction mixture is stirred at RT for 2 h. It is then added to 40 ml of NH₄Cl solution. After extraction with ethyl acetate, the organic phase is washed with water and sat. brine, dried over Na₂SO₄, filtered, concentrated and dried in vacuo. 2-Methoxy-8,9-dihydro-7H-benzocyclohepten-6-yl nonafluorobutane-1-sulphonate is obtained quantitatively.

¹H-NMR (CDCl₃) δ in ppm: 1.98 (m, 2H, CH₂), 2.77 (m, 2H, CH₂), 2.83 (m, 2H, CH₂), 3.80 (s, 3H, OCH₃), 6.55 (s, 1H, —CH═), 6.66 (m, 1H, CH_{Ar}), 6.72 (m, 1H, CH_{Ar}), 7.09 (m, 1H, CH_{Ar}).

¹⁹F-NMR (CDCl₃) δ in ppm: −125.78 (m, 2F, CF₂), −120.83 (m, 2F, CF₂), −110.02 (m, 2F, CF₂), −80.58 (t, 3F, CF₃).

### 1.2. 5-Ethyl-2-methoxy-5,7,8,9-tetrahydrobenzocyclohepten-6-one

830 mg of 2-methoxy-5,7,8,9-tetrahydrobenzocyclohepten-6-one are dissolved in 4.5 ml of t-BuOH. Addit ion of 516 mg of KOt-Bu and 0.35 ml of ethyl iodide is followed by heating in a microwave reactor at 100° C. for 1 h. The reaction mixture is concentrated, and the residue is taken up in water and extracted with ether. The organic phase is washed with water until neutral, dried over Na₂SO₄ and concentrated. Purification by column chromatography on silica gel results in 5-ethyl-2-methoxy-5,7,8,9-tetrahydrobenzocyclohepten-6-one.

¹H-NMR (CDCl₃) δ in ppm: 0.92 (t, 3H, CH₃), 1.74 (m, 1H, CH₂), 1.91 (m, 1H, CH₂), 2.07 (m, 1H, CH₂), 2.24 (m, 1H, CH₂), 2.46-2.72 (m, 2H, CH₂), 2.80-2.99 (m, 2H, CH₂), 3.64 (t, 1H, CH), 3.80 (s, 3H, OCH₃), 6.69-6.77 (m, 2H, CH_{Ar}), 7.07 (m, 1H, CH_{Ar}).

### 1.2.1. 2-Methoxy-5-ethyl-8,9-dihydro-7H-benzocyclohepten-6-yl nonafluorobutane-1-sulphonate

1.12 g of 5-ethyl-2-methoxy-5,7,8,9-tetrahydrobenzocyclohepten-6-one are dissolved in 12 ml of toluene at 0° C. Addition of 2.75 ml of DBU is followed by addition of 3.0 ml of nonaflyl fluoride. The reaction mixture is stirred at RT for 2 h. It is then added to 120 ml of NH₄Cl solution. After extraction with ethyl acetate, the organic phase is washed with water and sat. brine, dried over Na₂SO₄, filtered, concentrated and dried in vacuo. 2-Methoxy-5-ethyl-8,9-dihydro-7H-benzocyclohepten-6-yl nonafluorobutane-1-sulphonate is obtained quantitatively.

¹H-NMR (CDCl₃) δ in ppm: 0.96 (t, 3H, CH₃), 2.18-2.30 (m, 4H, 2CH₂), 2.61 (q, 2H, CH₂), 2.80 (m, 2H, CH₂), 3.83 (s, 3H, OCH₃), 6.77-6.84 (m, 2H, CH_{Ar}), 7.06 (m, 1H, CH_{Ar}).

¹⁹F-NMR (CDCl₃) δ in ppm: −125.78 (m, 2F, CF₂), −120.82 (m, 2F, CF₂), −110.67 (m, 2F, CF₂), −80.57 (t, 3F, CF₃).

### 1.3. 5-Allyl-2-methoxy-5,7,8,9-tetrahydrobenzocyclohepten-6-one

500 mg of 2-methoxy-5,7,8,9-tetrahydrobenzocyclohepten-6-one are dissolved in 3 ml of t-BuOH. Addition of 310 mg of KOt-Bu and 0.22 ml of allyl bromide is followed by heating in a microwave reactor at 100° C. for 1 h. The reaction mixture is concentrated, and the residue is taken up with water and extracted with ether. The organic phase is washed with water until neutral, dried over Na₂SO₄ and concentrated. Purification by column chromatography on silica gel results in 5-allyl-2-methoxy-5,7,8,9-tetrahydrobenzocyclohepten-6-one.

¹H-NMR (CDCl₃) δ in ppm: 1.89 (m, 1H, CH₂), 2.08 (m, 1H, CH₂), 2.49-2.55 (m, 2H, CH₂), 2.61-2.70 (m, 1H, CH₂), 2.79-2.87 (m, 1H, CH₂), 2.92-3.02 (m, 2H, CH₂), 3.79 (s, 3H, OCH₃), 3.84 (t, 1H, CH), 5.12 (m, 2H, CH═CH₂), 5.77 (m, 1H, CH═CH₂), 6.69-6.77 (m, 2H, CH_{Ar}), 7.06 (m, 1H, CH_{Ar}).

### 1.3.1. 2-Methoxy-5-allyl-8,9-dihydro-7H-benzocyclohepten-6-yl nonafluorobutane-1-sulphonate

1.3.2. 405 mg of 5-allyl-2-methoxy-5,7,8,9-tetrahydrobenzocyclohepten-6-one are dissolved in 5 ml of toluene at 0° C. Addition of 0.94 ml of DBU is followed by addition of 1.04 ml of nonaflyl fluoride. The reaction mixture is stirred at RT for 2 h. It is then added to 60 ml of NH₄Cl solution. After extraction with ethyl acetate, the organic phase is washed with water, sat. brine, dried over Na₂SO₄, filtered, concentrated and dried in vacuo. 2-Methoxy-5-allyl-8,9-dihydro-7H-benzocyclohepten-6-yl nonafluorobutane-1-sulphonate is obtained quantitatively.

¹H-NMR (CDCl₃) δ in ppm: 2.22-2.33 (m, 4H, 2CH₂), 2.67 (m, 2H, CH₂), 3.35 (m, 2H, CH₂), 3.83 (s, 3H, OCH₃), 4.97-5.10 (m, 2H, CH═CH₂), 5.63-5.75 (m, 1H, CH═CH₂), 6.65-6.84 (m, 2H, CH_{Ar}), 7.25 (m, 1H, CH_{Ar}).

¹⁹F-NMR (CDCl₃) δ in ppm: −125.77 (m, 2F, CF₂), −120.81 (m, 2F, CF₂), −110.53 (m, 2F, CF₂), −80.57 (t, 3F, CF₃).

### 1.4. 5,5-Diethyl-2-methoxy-5,7,8,9-tetrahydrobenzocyclohepten-6-one

300 mg of 2-methoxy-5,7,8,9-tetrahydrobenzocyclohepten-6-one are dissolved in 3 ml of t-BuOH. Addition of 375 mg of KOt-Bu and 0.26 ml of ethyl iodide is followed by heating in a microwave reactor at 100° C. for 1 h. The reaction mixture is concentrated, and the residue is taken up with water and extracted with ether. The organic phase is washed with water until neutral, dried over Na₂SO₄ and concentrated. Purification by column chromatography on silica gel results in 5,5-diethyl-2-methoxy-5,7,8,9-tetrahydrobenzocyclohepten-6-one.

¹H-NMR (CDCl₃) δ in ppm: 0.71 (m, 6H, 2CH₃), 1.77-2.05 (m, 6H, 3CH₂), 2.44 (m, 2H, CH₂), 2.67 (m, 2H, CH₂), 3.82 (s, 3H, OCH₃), 6.70 (m, 1H, CH_{Ar}), 6.80 (m, 1H, CH_{Ar}), 7.18 (m, 1H, CH_{Ar}).

### 1.4.1. 2-Methoxy-5,5-diethyl-8,9-dihydro-7H-benzocyclohepten-6-yl nonafluorobutane-1-sulphonate

145 mg of 5,5-diethyl-2-methoxy-5,7,8,9-tetrahydrobenzocyclohepten-6-one are dissolved in 5 ml of THF. Under argon at 0° C., 0.44 ml of 2M LDA solution is added. After stirring at 0° C. for 15 min, 0.16 ml of nonaflyl fluoride is added dropwise. The reaction mixture is then stirred at RT for 3 h. It is subsequently added to 20 ml of NH₄Cl solution. After extraction with ethyl acetate, the organic phase is washed with water and sat. brine, dried over Na₂SO₄, filtered, concentrated and dried in vacuo. 2-Methoxy-5,5-diethyl-8,9-dihydro-7H-benzocyclohepten-6-yl nonafluorobutane-1-sulphonate is obtained.

¹⁹F-NMR (CDCl₃) δ in ppm: −125.9 (m, 2F, CF₂), −121.6 (m, 2F, CF₂), −114.4 (m, 2F, CF₂), −80.8 (t, 3F, CF₃).

### 1.5. 2-Methoxy-6-(4-methoxyphenyl)-8,9-dihydro-7H-benzocyclohepten-5-yl nonafluorobutane-1-sulphonate

1.0 g of 2-methoxy-6-(4-methoxyphenyl)-6,7,8,9-tetrahydrobenzocyclohepten-5-one is dissolved in 15 ml of toluene. At 0° C., 1.8 ml of DBU and 2 ml of nonaflyl fluoride are added. After stirring at RT for 3 h, the reaction mixture is added to 40 ml of NH₄Cl solution, the product is extracted with ethyl acetate, and the org. phase is washed with water and sat. brine. It is dried over Na₂SO₄ and then filtered and concentrated. 2-Methoxy-6-(4-methoxyphenyl)-8,9-dihydro-7H-benzocyclohepten-5-yl nonafluorobutane-1-sulphonate is obtained.

¹H-NMR (CDCl₃) δ in ppm: 2.25 (m, 2H, CH₂), 2.39 (m, 2H, CH₂), 2.85 (m, 2H, CH₂), 3.84 (s, 3H, OCH₃), 3.86 (s, 3H, OCH₃), 6.70-6.98 (m, 4H, CH_{Ar}), 7.37-7.46 (m, 3H, CH_{Ar}).

### 2. Preparation of the Test Compounds

### Variant A

### 3-Methoxy-8-(4-methoxyphenyl)-9-vinyl-6,7-dihydro-5H-benzocycloheptene

0.63 ml of 10% strength t-Bu₃P in n-hexane and 0.25 ml of triethylvinyltin are added to a mixture of 425 mg of 2-methoxy-6-(4-methoxyphenyl)-8,9-dihydro-7H-benzocyclohepten-5-yl nonafluorobutanel-sulphonate, 240 mg of CsF, Pd(PPh₃)₄ in 2 ml of dioxane under argon. The reaction mixture is heated at 120° C. in a microwave reactor for 2 h. The reaction mixture is filtered through Celite and concentrated, and the product is purified by column chromatography. 3-Methoxy-8-(4-methoxyphenyl)-9-vinyl-6,7-dihydro-5H-benzocycloheptene is obtained.

¹H-NMR (CDCl₃) δ in ppm: 2.10 (m, 2H, CH₂), 2.35 (m, 2H, CH₂), 2.67 (m, 2H, CH₂), 3.88 (s, 6H, 2OCH₃), 4.98-5.09 (m, 2H, CH═CH₂), 6.62-6.73 (m, 1H, CH═CH₂), 6.78-6.98 (m, 4H, CH_{Ar}), 7.24-7.33 (m, 3H, CH_{Ar}).

### Variant B

### 6-(4-Hydroxyphenyl)-5-vinyl-8,9-dihydro-7H-benzocyclohepten-2-ol

100 mg of 3-methoxy-8-(4-methoxyphenyl)-9-vinyl-6,7-dihydro-5H-benzocycloheptene are introduced into 4.5 ml of CH₂Cl₂ at −10° C. 3 ml of 1M BBr₃ solution in CH₂Cl₂ are added dropwise, and the mixture is stirred at RT for 1 h. The reaction mixture is added to 40 ml of water and extracted with ethyl acetate. The org. phase is washed with sat. brine, dried over Na₂SO₄, filtered and concentrated. Purification by column chromatography results in 6-(4-Hydroxy-phenyl)-5-vinyl-8,9-dihydro-7H-benzocyclohepten-2-ol: C₁₉H₁₈O₂: M⁺=278 m/e.

¹H-NMR (DMSO-d₆) δ in ppm: 9.19 (s, 1H, OH), 9.32 (s, 1H, OH).

### 6-(4-Hydroxy-phenyl)-6-methyl-6,7,8,9-tetrahydro-5H-benzocyclohepten-2-ol

¹H-NMR (CDCl₃) δ in ppm: 1.06 (s, 3H, CH₃), 1.60-1.87 (m, 3H, CH₂), 2.05-2.16 (m, 1H, CH₂), 2.66-2.85 (m, 3H, CH₂), 3.31-3.36 (d, 1H, CH₂), 4.58 (s, 1H, OH), 4.65 (s, 1H, OH), 6.52-6.59 (m, 2H, CH_{Ar}), 6.76-6.81 (m, 2H, CH_{Ar}), 6.87-6.90 (m, 1H, CH_{Ar}), 7.29-7.34 (m, 2H, CH_{Ar}).

### 5-Ethyl-6-(4-hydroxy-2-methyl-phenyl)-8,9-dihydro-7H-benzocyclohepten-2-ol

¹H-NMR (DMSO-d₆). δ in ppm: 0.63-0.67 (t, 3H, CH₃), 1.71-1.76 (m, 1H, CH₂), 1.85-2.02 (m, 4H, CH₂), 2.06 (s, 3H, CH₃), 2.11-2.20 (m, 1H, CH₂), 2.44-2.50 (m, 1H, CH₂), 2.56-2.63 (m, 1H, CH₂), 6.51-6.55 (m, 1H, CH_{Ar}), 6.59-6.63 (m, 3H, CH_{Ar}), 6.84-6.86 (d, 1H, CH_{Ar}), 7.05-7.08 (d, 1H, CH_{Ar}), 9.13 (s, 1H, OH), 9.23 (s, 1H, OH).

### Variant C

### 6-(4-Hydroxyphenyl)-8,9-dihydro-7H-benzocyclohepten-2-ol

150 mg of 2-methoxy-8,9-dihydro-7H-benzocyclohepten-6-yl nonafluorobutane-1-sulphonate and 70 mg of p-methoxyphenylboronic acid are introduced into 1.5 ml of toluene and 1.5 ml of ethanol under argon. 22 mg of Pd(PPh₃)₄ and 0.61 ml of a 1M Na₂CO₃ solution are added. The mixture is then heated in a microwave reactor at 120° C. for 15 min. The reaction mixture is concentrated and taken up in 4 ml of CH₂Cl₂. At 0° C., 3 ml of 1M BBr₃ solution are added, and the mixture is stirred at RT for 2 h. The reaction mixture is added to 40 ml of water and extracted with ethyl acetate. The org. phase is washed with sat. brine, dried over Na₂SO₄, filtered and concentrated. Purification by column chromatography results in 6-(4-hydroxyphenyl)-8,9-dihydro-7H-benzocyclohepten-2-ol.

¹H-NMR (DMSO-d₆). δ in ppm: 2.03 (m, 2H, CH₂), 2.53 (m, 2H, CH₂), 2.65 (m, 2H, CH₂), 6.48-6.61 (m, 3H, CH_{Ar}), 6.74 (m, 2H, CH_{Ar}), 7.04 (m, 1H, CH_{Ar}), 7.32 (m, 2H, CH_{Ar}), 9.32 (s, 1H, OH), 9.38 (s, 1H, OH).

### Variant D

### 5-Ethyl-6-(4-hydroxyphenyl)-8,9-dihydro-7H-benzocyclohepten-2-ol

20 mg of 6-(4-hydroxyphenyl)-5-vinyl-8,9-dihydro-7H-benzocyclohepten-2-ol and 10 mg of Pd/C (5%) are put into 10 ml of methanol. Hydrogenation is carried out under atmospheric pressure at RT. The solution is filtered and concentrated. 5-Ethyl-6-(4-hydroxyphenyl)-8,9-dihydro-7H-benzocyclohepten-2-ol is obtained: C₁₉H₂₀O₂: M⁺=280 m/e.

### Variant E

### 6-(4-Hydroxyphenyl)-5-propyl-6,7,8,9-tetrahydro-5H-benzocyclohepten-2-ol

20 mg of 5-allyl-6-(4-hydroxyphenyl)-8,9-dihydro-7H-benzocyclohepten-2-ol and 10 mg of Pd/C (5%) are put into 10 ml of methanol. Hydrogenation is carried out under 7.5 bar at RT. The solution is filtered and concentrated. 6-(4-Hydroxyphenyl)-5-propyl-6,7,8,9-tetrahydro-5H-benzocyclohepten-2-ol is obtained: C₂₀H₂₄O₂: M⁺=296 m/e.

### Variant F

### 9-Allyl-3-methoxy-8-(4-methoxyphenyl)-6,7-dihydro-5H-benzocycloheptene

400 mg of 2-methoxy-5-allyl-8,9-dihydro-7H-benzocyclohepten-6-yl nonafluorobutane-1-sulphonate and 170 mg of p-methoxyphenylboronic acid are introduced into 3.7 ml of toluene and 3.7 ml of ethanol under argon. 55 mg Pd(PPh₃)₄ and 1.5 ml of a 1M Na₂CO₃ solution are added. The mixture is then heated in a microwave reactor at 120° C. for 15 min. The reaction mixture is concentrated, and the product is chromatographed. 9-Allyl-3-methoxy-8-(4-methoxyphenyl)-6,7-dihydro-5H-benzocycloheptene is obtained.

¹H-NMR (CDCl₃) δ in ppm: 2.07-2.20 (m, 4H, 2CH₂), 2.67 (m, 2H, CH₂), 3.20 (m, 2H, CH₂), 3.84 (s, 6H, 2OCH₃), 4.88 (m, 2H, CH═CH₂), 5.67 (m, 1H. CH═CH₂), 6.75-6.83 (m, 4H, CH_{Ar}), 7.22-7.30 (m, 3H, CH_{Ar}).

### Variant G

### 9-Ethyl-3-methoxy-8-(4-methoxyphenyl)-6,7-dihydro-5H-benzocycloheptene

400 mg of 2-methoxy-5-ethyl-8,9-dihydro-7H-benzocyclohepten-6-yl nonafluorobutane-1-sulphonate and 170 mg of p-methoxyphenylboronic acid are introduced into 3.7 ml of toluene and 3.7 ml of ethanol under argon. 55 mg Pd(PPh₃)₄ and 1.5 ml of a 1M Na₂CO₃ solution are added. The mixture is then stirred at RT until conversion is complete. The reaction mixture is concentrated and the product is chromatographed. 9-Ethyl-3-methoxy-8-(4-methoxyphenyl)-6,7-dihydro-5H-benzocycloheptene is obtained.

¹H-NMR (CDCl₃). δ in ppm: 0.83 (t, 3H, CH₃), 2.06-2.14 (m, 4H, 2CH₂), 2.43 (q, 2H, CH₂), 2.65 (m, 2H, CH₂), 3.19 (m, 2H, CH₂), 3.78-3.84 (2s+m, 8H, CH₂+2OCH₃), 6.76-6.93 (m, 4H, CH_{Ar}), 7.17-7.25 (m, 3H, CH_{Ar}).

### Variant H

### 5-Ethyl-6-(4-hydroxyphenyl)-8,9-dihydro-7H-benzocyclohepten-2-ol

50 mg of 9-ethyl-3-methoxy-8-(4-methoxyphenyl)-6,7-dihydro-5H-benzocycloheptene are dissolved in 3.5 ml of CH₂Cl₂. Addition of 230 mg of tetrabutylammonium iodide is followed by cooling to −60° C. and addition of 1.25 ml of 1M BCl₃ solution in CH₂Cl₂. After 1 h at 0° C., the mixture is stirred at RT for a further hour. Then 10 ml of sat. NH₄Cl solution are added, followed after phase separation by concentration to dryness and purification by column chromatography. 5-Ethyl-6-(4-hydroxy-phenyl)-8,9-dihydro-7H-benzocyclohepten-2-ol is obtained: C₁₉H₂₀O₂, M⁺=280 m/e.

¹H-NMR (DMSO-d₆). δ in ppm: 0.68-0.73 (t, 3H, CH₃), 1.88-2.00 (m, 4H, CH₂), 2.25-2.35 (m, 2H, 2CH₂), 2.46-2.52 (m(überlg.), 2H, CH₂), 6.58-6.63 (m, 2H, CH_{Ar}), 6.68-6.74 (m, 2H, CH_{Ar}), 6.98-7.09 (m, 3H, CH_{Ar}), 9.19-9.31 (2s (br.), 2H, 20H).

### Variant I

### 6-(4-Hydroxyphenyl)-5-propyl-8,9-dihydro-7H-benzocyclohepten-2-ol

60 mg of 9-allyl-3-methoxy-8-(4-methoxyphenyl)-6,7-dihydro-5H-benzocycloheptene are introduced into 4 ml of toluene under argon. Then 1.5 ml of 1.5M DIBAH solution in toluene are added. The reaction mixture is boiled under reflux for 2 h. The cooled reaction mixture is added to 30 ml of ice-water. Extraction with ethyl acetate is followed by drying over Na₂SO₄, filtration and concentration. Purification by column chromatography results in 6-(4-hydroxyphenyl)-5-propyl-8,9-dihydro-7H-benzocyclohepten-2-ol: C₂₀H₂₂O₂, M⁺=294 m/e.

¹H-NMR (DMSO-d₆). δ in ppm: 0.67 (t, 3H, CH₃), 1.17 (m, 2H, CH₂), 1.80-2.22 (m(overlap), 4H, 2CH₂), 2.31 (m, 2H, CH₂), 2.51 (m(overlap), 2H, CH₂), 6.60-6.78 (m, 4H, CH_{Ar}), 7.00-7.13 (m, 3H, CH_{Ar}), 9.22-9.38 (2s (br.), 2H, 20H).

### Variant J

### 2-Methoxy-6-(4-methoxyphenyl)-6-allyl-6,7,8,9-tetrahydrobenzocyclohepten-5-one

200 mg of 2-methoxy-6-(4-methoxyphenyl)-6,7,8,9-tetrahydrobenzocyclohepten-5-one are dissolved in 3 ml of tert-butanol and 1.8 ml of allyl bromide. Addition of 265 mg of KOt.Bu is followed by stirring at RT for 3 h. After the reaction is complete, the mixture is diluted with 15 ml of water. Phase separation is followed by back-extraction with ethyl acetate, and the combined org. phases are washed with brine, dried over Na₂SO₄, filtered and concentrated.

Purification by column chromatography on silica gel results in 2-methoxy-6-(4-methoxyphenyl)-6-allyl-6,7,8,9-tetrahydrobenzocyclohepten-5-one: C₂₂H₂₄O₃, M⁺=336 m/e.

¹H-NMR (CDCl₃) δ in ppm: 1.80 (m, 2H, CH₂), 2.05 (m, 1H, CH₂), 2.20 (m, 1H, CH₂), 2.39-2.59 (m, 3H, CH₂), 2.92 (m, 2H, CH₂), 3.80 (s, 3H, OCH₃), 3.83 (s, 3H, OCH₃), 4.95-5.07 (m, 2H, ═CH₂), 5.60 (m, H, ═CH), 6.57 (m, 1H, CH_{Ar}), 6.80 (m, 1H, CH_{Ar}), 6.86 (m, 2H, CH_{Ar}), 7.24 (m, 2H, CH_{Ar}), 7.57 (m, 1H, CH_{Ar}).

### Variant K

### 2-Methoxy-6-(4-methoxyphenyl)-6-propyl-6,7,8,9-tetrahydro-5H-benzocycloheptene

180 mg of 2-methoxy-6-(4-methoxyphenyl)-6-propyl-6,7,8,9-tetrahydrobenzocyclohepten-5-one are introduced into 15 ml of CH₂Cl₂ and 8 ml of triethylsilane under argon. At −40° C., 15 ml of boron trifluoride etherate are added dropwise. The mixture is then stirred at RT for 3 h.

While cooling, 40 ml of 10% strength K₂CO₃ solution are slowly added. After stirring at RT for 8 h, the phases are separated and, after extraction with ethyl acetate, the combined org. phases are washed with water and sat. brine. Drying over Na₂SO₄ is followed by filtration and concentration. 2-Methoxy-6-(4-methoxyphenyl)-6-propyl-6,7,8,9-tetrahydro-5H-benzocycloheptene is obtained: C₂₂H₂₈O₂, M⁺=324 m/e.

¹H-NMR (CDCl₃) δ in ppm: 0.70 (t, 3H, CH₃), 0.98 (m, 2H, CH₂), 1.41 (m, 2H, CH₂), 1.75 (m, 2H, CH₂), 1.84-2.13 (m, 2H, CH₂), 2.75 (m, 2H, CH₂), 2.95-3.21 (m, 2H, CH₂), 3.78-3.84 (2s, 6H, OCH₃), 6.56-6.66 (m, 3H, CH_{Ar}), 6.85 (m, 2H, CH_{Ar}), 7.02 (m, 1H, CH_{Ar}), 7.30 (m, 2H, CH_{Ar}).

### Variant L

### 2-Methoxy-6-(4-methoxyphenyl)-6-methyl-6,7,8,9-tetrahydrobenzocyclohepten-5-one

4.19 g of 2-methoxy-6-(4-methoxyphenyl)-6,7,8,9-tetrahydrobenzocyclohepten-5-one are dissolved in 44 ml of methyl iodide and, over the course of 45 min, 5.55 g of potassium tert-butoxide are added in portions (internal temperature +20 to +36° C.), immediately pale yellow-coloured suspension. After the addition is complete, 90 ml of tert-butanol are added to the suspension, and the mixture is stirred at RT for 20 h. It is added to 300 ml of water/ice, extracted with 250 ml of ethyl acetate and back-extracted twice with 150 ml of ethyl acetate each time. The organic phases are washed once with saturated NaCl solution, dried over Na₂SO₄, filtered and concentrated: 4.65 g of crude product. The crude product is dissolved in ethyl acetate, absorbed on silica gel, dried, put on a 50 g Flashmaster Si column and chromatographed with hexane/ethyl acetate (0 to 20% ethyl acetate): 3.5 g of 2-methoxy-6-(4-methoxyphenyl)-6-methyl-6,7,8,9-tetrahydrobenzocyclohepten-5-one: C₂₀H₂₂O₃, MH⁺=311 m/e.

¹H-NMR (DMSO). δ in ppm: 1.36 (s, 3H), 1.71 (m, 3H), 2.22 (m, 1H), 2.53 (m, 2H), 3.68 (s, 3H), 3.75 (s, 3H), 6.71 (d, 1H), 6.83 (m, 3H), 7.12 (m, 2H), 7.37 (d, 1H).

### Variant M

### 2-Methoxy-6-(4-methoxyphenyl)-6-methyl-5-methylene-6,7,8,9-tetrahydro-5H-benzocycloheptene

1.73 g of methyltriphenylphosphonium bromide are dissolved in 10 ml of DMSO and then 211 mg of sodium hydride are added in portions. This is heated to a bath temperature of 60° C. Then 100 mg of 2-methoxy-6-(4-methoxyphenyl)-6-methyl-6,7,8,9-tetrahydrobenzocyclohepten-5-one dissolved in 4 ml of DMSO are slowly added, and the mixture is left to stir at 60° C. for 3 h. The reaction mixture is allowed to cool to RT and slowly poured into about 15 ml of water. It is then extracted three times with 30 ml of ethyl acetate each time, washed once with saturated NaCl solution, dried over sodium sulphate, filtered and concentrated. The crude product is dissolved in a little ethyl acetate and absorbed on silica gel. It is subsequently put on a 25 g Si column and purified in a Flash Master. Hexane/ethyl acetate (0-10%) is used as mobile phase mixture: 37.5 g of 2-methoxy-6-(4-methoxyphenyl)-6-methyl-5-methylene-6,7,8,9-tetrahydro-5H-benzocycloheptene: C₂₁H₂₄O₂, MH⁺=309 m/e.

¹H-NMR (CDCl₃). δ in ppm: 1.39 (s, 3H), 1.66 (m, 2H), 1.81 (m, 1H), 2.12 (m, 1H), 2.65 (m, 2H), 3.70 (s, 3H), 3.71 (s, 3H), 5.02 (d, 1H), 5.12 (bs, 1H), 6.64 (d, 1H), 6.70 (dd, 1H), 6.80 (m, 2H), 7.04 (d, 1H), 7.23 (m, 2H).

### Variant N

### 5-Ethyl-2-methoxy-6-(4-methoxyphenyl)-6-methyl-6,7,8,9-tetrahydro-5H-benzocyclohepten-5-ol

204 mg of 2-methoxy-6-(4-methoxyphenyl)-6-methyl-6,7,8,9-tetrahydrobenzocyclohepten-5-one are dissolved in 5 ml of THF under argon and cooled to −36° C. in a dry ice bath, 1 ml of ethyllithium solution (1.7 molar in di-tert-butyl ether) is added, and the mixture is stirred at −36 to −30° C. for 30 min. The reaction solution is then slowly warmed to RT. The reaction mixture is cooled in an ice bath, 10 ml of sat. NH₄Cl solution are cautiously added, and the mixture is diluted with water and extracted three times with 30 ml of ethyl acetate each time. The organic phases are dried over Na₂SO₄, filtered and concentrated: 267 mg of viscous oily crude product. The crude product is absorbed on silica gel, dried, put on a 5 g Flashmaster Si column and chromatographed with hexane/ethyl acetate (0 to 10% ethyl acetate).

128 mg of 5-ethyl-2-methoxy-6-(4-methoxyphenyl)-6-methyl-6,7,8,9-tetrahydro-5H-benzocyclohepten-5-ol: C₂₂H₂₈O₃, MH⁺=341 m/e.

### Variant O

### 5-Ethylidene-2-methoxy-6-(4-methoxyphenyl)-6-methyl-6,7,8,9-tetrahydro-5H-benzocycloheptene

110 mg of 5-ethyl-2-methoxy-6-(4-methoxyphenyl)-6-methyl-6,7,8,9-tetrahydro-5H-benzocyclohepten-5-ol are dissolved in 2.5 ml of toluene under argon, 28 mg of p-toluenesulphonic acid monohydrate are added, and the mixture is stirred in an oil bath at a bath temperature of 600 under argon for 1 h. The reaction mixture is cooled to RT, diluted with 20 ml of ethyl acetate, put into 10 ml of sat. NaHCO₃ solution and shaken, and the aqueous phase is back-extracted twice with 20 ml of ethyl acetate each time. The organic phases are washed once with sat. NaCl solution, dried over Na₂SO₄, filtered and concentrated: 131 mg of oily crude product. The crude product is dissolved in a little ethyl acetate, absorbed on silica gel, dried, put on a 5 g Flashmaster Si column and chromatographed with hexane/ethyl acetate (0 to 10% ethyl acetate): 92 mg of 5-ethylidene-2-methoxy-6-(4-methoxyphenyl)-6-methyl-6,7,8,9-tetrahydro-5H-benzocycloheptene: C₂₂H₂₆O₂, MH⁺=323 m/e.

### Variant P

### 5-Ethyl-2-methoxy-6-(4-methoxyphenyl)-6-methyl-6,7,8,9-tetrahydro-5H-benzocycloheptene

81 mg of 5-ethylidene-2-methoxy-6-(4-methoxyphenyl)-6-methyl-6,7,8,9-tetrahydro-5H-benzocycloheptene are dissolved in 2.5 ml of ethyl acetate, and 36 mg of palladium-activated carbon (10%) are added. The reaction flask is evacuated several times, flushed with hydrogen and then stirred under hydrogen for 24 h. The catalyst is filtered off and washed with ethyl acetate, and the filtrate is concentrated: 76 mg of crude product. The crude product is dissolved in a little ethyl acetate, absorbed on silica gel, dried, put on a 5 g Flashmaster Si column and chromatographed with hexane/ethyl acetate (0 to 10% ethyl acetate): 74 mg of 5-ethyl-2-methoxy-6-(4-methoxyphenyl)-6-methyl-6,7,8,9-tetrahydro-5H-benzocycloheptene: C₂₂H₂₈O₂, MH⁺=325 m/e

¹H-NMR (DMSO). δ in ppm: 0.38 (m, 3H), 0.91 (s, 3H), 1.01 (m, 1H), 1.62 (m, 2H), 1.77 (m, 1H), 1.85 (m, 1H), 2.398 (m, 1H), 2.60 (m, 2H), 2.91 (t, 1H), 3.69 (s, 3H), 3.70 (s, 3H), 6.64 (m, 2H), 6.85 (d, 2H), 7.04 (d, 1H), 7.39 (d, 2H).

### Variant Q

### 5-Ethyl-6-(4-hydroxyphenyl)-6-methyl-6,7,8,9-tetrahydro-5H-benzocyclohepten-2-ol

427 mg of 5-ethyl-2-methoxy-6-(4-methoxyphenyl)-6-methyl-6,7,8,9-tetrahydro-5H-benzocycloheptene are dissolved in 25 ml of CH₂Cl₂ under argon and cooled to −71° C., and 3.95 ml of boron tribromide solution (1 molar in CH₂Cl₂) are added dropwise, and the mixture is stirred at −71° C. for 40 min and, after removal of the cooling bath, slowly warmed to RT. The reaction mixture is stirred into 60 ml of sat. NaHCO₃ solution/ice and stirred for 20 min, the organic phase is separated off, and the aqueous phase is back-extracted twice with CH₂Cl₂. The combined organic phases are washed once with sat. NaCl solution, dried over Na₂SO₄, filtered and concentrated: 439 mg of crude product. The crude product is dissolved in a little ethyl acetate, absorbed on silica gel, dried, put on a 20 g Flashmaster Si column and chromatographed with hexane/ethyl acetate (0 to 50% ethyl acetate): 418 mg. The 4 isomers are separated by chiral preparative HPLC: 27.2 mg, 23.4 mg, 152.3 mg and 149.4 mg of 5-ethyl-6-(4-hydroxyphenyl)-6-methyl-6,7,8,9-tetrahydro-5H-benzocyclohepten-2-ol: C₂₀H₂₄O₂, MH⁺=297 m/e.

¹H-NMR (DMSO). δ in ppm: 0.67 (t, 3H), 0.91 (m, 4H), 1.25 (m, 3H), 1.51 (m, 2H), 1.66 (m, 1H), 2.50 (m, 1H), 3.09 (bs, 1H), 6.60 (m, 4H), 6.89 (d, 1H), 7.21 (bs, 2H), 8.98 (s, 1H), 9.08 (s, 1H).

¹H-NMR (DMSO). δ in ppm: 0.67 (t, 3H), 0.91 (m, 4H), 1.25 (m, 3H), 1.51 (m, 2H), 1.66 (m, 1H), 2.50 (m, 1H), 3.09 (bs, 1H), 6.60 (m, 4H), 6.89 (d, 1H), 7.21 (bs, 2H), 8.98 (s, 1H), 9.08 (s, 1H).

¹H-NMR (DMSO) δ in ppm: 0.36 (s, 3H), 0.89 (s, 3H), 1.02 (m, 1H), 1.60 (m, 2H), 1.74 (m, 1H), 1.84 (m, 1H), 2.37 (m, 1H), 2.51 (m, 2H), 2.86 (t, 1H), 6.44 (m, 2H), 6.67 (d, 2H), 6.86 (d, 1H), 7.25 (d, 2H), 8.99 (s, 1H), 9.09 (s, 1H).

¹H-NMR (DMSO) δ in ppm: 0.36 (s, 3H), 0.89 (s, 3H), 1.02 (m, 1H), 1.60 (m, 2H), 1.74 (m, 1H), 1.84 (m, 1H), 2.37 (m, 1H), 2.51 (m, 2H), 2.86 (t, 1H), 6.44 (m, 2H), 6.67 (d, 2H), 6.86 (d, 1H), 7.25 (d, 2H), 8.99 (s, 1H), 9.09 (s, 1H).

### Variant R

### 2-Hydroxy-6-(4-hydroxyphenyl)-6,7,8,9-tetrahydrobenzocyclohepten-5-one

7.5 ml of 1 molar boron tribromide solution (in CH₂Cl₂) are cooled to −38° C. under argon; 0.87 ml of 2,6-dimethylpyridine is added dropwise at −38 to −31° C., and the mixture is stirred at −31 to 27° C. for 15 min. Then 148.2 mg of 2-methoxy-6-(4-methoxyphenyl)-6,7,8,9-tetrahydrobenzocyclohepten-5-one are added as solid, and the reaction mixture is slowly warmed to RT and stirred at RT for 20 h. The reaction mixture is diluted with 10 ml of dichloromethane, cooled to +3° C. in an ice bath, and 5 ml of water are added dropwise. The cloudy mixture is diluted with 40 ml of CH₂Cl₂ and 40 ml of H₂O, shaken and back-extracted twice with CH₂Cl₂. The organic phases are washed twice with water, dried over Na₂SO₄, filtered and concentrated: 156 mg. The aqueous phases are back-extracted three times with 40 ml of ethyl acetate, and the organic phases are washed once with water and once with saturated NaCl solution, dried over Na₂SO₄, filtered and concentrated: 35 mg. The extracts are combined, dissolved in THF/methanol, absorbed on silica gel, dried, put on a 20 g Flashmaster Si column and chromatographed with hexane/ethyl acetate (0 to 100% ethyl acetate): 124 mg of 2-hydroxy-6-(4-hydroxyphenyl)-6,7,8,9-tetrahydrobenzocyclohepten-5-one: C₁₇H₁₆O₃, MH⁺=269 m/e.

¹H-NMR (DMSO). δ in ppm: 1.56 (m, 1H), 1.90 (m, 2H), 2.04 (m, 1H), 2.78 (m, 1H), 3.05 (m, 1H), 3.97 (m, 1H), 6.64 (m, 4H), 6.97 (d, 2H), 7.41 (d, 1H), 9.16 (s, 1H), 10.04 (s, 1H).

### Variant S

### 6-(4-Hydroxyphenyl)-6-vinyl-6,7,8,9-tetrahydro-5H-benzocyclohepten-2-ol

### 6-(2-Benzyloxyethyl)-2-methoxy-6-(4-methoxyphenyl)-6,7,8,9-tetrahydrobenzocyclohepten-5-one

16 g of 2-methoxy-6-(4-methoxyphenyl)-6,7,8,9-tetrahydrobenzocyclohepten-5-one were introduced into 140 ml of tert-butanol at RT. Addition of 66 ml of (2-bromo-ethoxymethyl)benzene and 21.2 g of potassium tert-butoxide was followed by stirring at RT for 17 h. The reaction mixture was mixed with H₂O and extracted with ethyl acetate. The combined org. phases were washed with H₂O, dried over Na₂SO₄, filtered and concentrated. Purification by column chromatography on silica gel resulted in 6-(2-benzyloxyethyl)-2-methoxy-6-(4-methoxyphenyl)-6,7,8,9-tetrahydrobenzocyclohepten-5-one: C₂₈H₃₀O₄: MH⁺=431 m/e (16.2 g, 70%).

¹H-NMR (CDCl₃) δ in ppm: 1.78 (m, 2H, CH₂), 2.09 (m, 2H, CH₂), 2.26 (m, 1H, CH₂), 2.42 (m, 1H, CH₂), 2.54 (m, 2H, CH₂), 3.44 (m, 2H, CH₂), 3.80 (s, 3H, CH₃), 3.83 (s, 3H, CH₃), 4.36 (d, 1H, CH₂), 4.41 (d, 1H, CH₂), 6.57 (d, 1H, CH_{Ar}), 6.75 (dd, 1H, CH_{Ar}), 6.85 (m, 2H, CH_{Ar}), 7.30 (m, 7H, CH_{Ar}), 7.51 (d, 1H, CH_{Ar}).

### 6-(2-Benzyloxyethyl)-2-methoxy-6-(4-methoxyphenyl)-6,7,8,9-tetrahydro-5H-benzocyclohepten-5-ol

8.28 g of 6-(2-benzyloxyethyl)-2-methoxy-6-(4-methoxyphenyl)-6,7,8,9-tetrahydrobenzocyclohepten-5-one were introduced into a mixture of 52 ml of methanol and 52 ml of tetrahydrofuran and cooled to −10° C. 3.64 g of sodium borohydride were added in portions over the course of 15 min and, after the addition was complete, the reaction mixture was stirred at RT for 30 min. After addition of H₂O, the reaction mixture was concentrated in vacuo, and the resulting residue was extracted with ethyl acetate. The combined org. phases were washed with H₂O, dried over Na₂SO₄, filtered and concentrated. Purification by column chromatography on silica gel resulted in 6-(2-benzyloxyethyl)-2-methoxy-6-(4-methoxyphenyl)-6,7,8,9-tetrahydro-5H-benzocyclohepten-5-ol: C₂₈H₃₂O₄: [M-OH]⁺=415 m/e (6.25 g, 75%).

### 6-(2-Hydroxyethyl)-2-methoxy-6-(4-methoxyphenyl)-6,7,8,9-tetrahydro-5H-benzocyclohepten-5-ol

1.24 g of palladium on carbon (10%) were introduced under argon. A solution of 12 g of 6-(2-benzyloxyethyl)-2-methoxy-6-(4-methoxyphenyl)-6,7,8,9-tetrahydro-5H-benzocyclohepten-5-ol in 600 ml of methanol was added, and the mixture was stirred at RT under an H₂ pressure of 1 bar for 7 h. The reaction mixture was filtered through Celite and concentrated. Purification by column chromatography on silica gel resulted in 6-(2-hydroxyethyl)-2-methoxy-6-(4-methoxyphenyl)-6,7,8,9-tetrahydro-5H-benzocyclohepten-5-ol (9.36 g, 99%).

¹H-NMR (DMSO-d₆). δ in ppm: 1.65 (m, 6H, CH₂), 2.56 (m, 1H, CH₂), 2.83 (m, 1H, CH₂), 3.05 (m, 1H, CH₂), 3.20 (m, 1H, CH₂), 3.72 (s, 3H, CH₃), 3.73 (s, 3H, CH₃), 4.05 (s, 1H, OH), 4.55 (m, 1H, CH), 4.84 (s, 1H, OH), 6.65 (m, 2H, CH_{Ar}), 6.86 (m, 2H, CH_{Ar}), 7.15 (d, 1H, CH_{Ar}), 7.41 (m, 2H, CH_{Ar}).

### 2-[2-Methoxy-6-(4-methoxyphenyl)-6,7,8,9-tetrahydro-5H-benzocyclohepten-6-yl]-ethanol

9.36 g of 6-(2-hydroxyethyl)-2-methoxy-6-(4-methoxyphenyl)-6,7,8,9-tetrahydro-5H-benzocyclohepten-5-ol were introduced into 203 ml of dichloromethane, and 20.3 ml of triethylsilane were added. The reaction mixture was cooled to −40° C., and 16.3 ml of boron trifluoride diethyl etherate were added. It was allowed to warm slowly to 0° C. and was stirred at 0° C. for 2.5 h. Addition of sat. NaHCO₃ solution was followed by phase separation. The org. phase was washed with H₂O, dried over Na₂SO₄, filtered and concentrated. Purification by column chromatography on silica gel resulted in 2-[2-methoxy-6-(4-methoxyphenyl)-6,7,8,9-tetrahydro-5H-benzocyclohepten-6-yl]-ethanol (8.46 g, 95%, as mixture with 8-methoxy-3a-(4-methoxyphenyl)-3,3a,4,5,6,10b-hexahydro-2H-benzo[6,7]cyclohepta[1,2-b]furan). This mixture was directly reacted further.

### 6-(2-Hydroxyethyl)-6-(4-hydroxyphenyl)-6,7,8,9-tetrahydro-5H-benzocyclohepten-2-ol

8.46 g of 2-[2-methoxy-6-(4-methoxyphenyl)-6,7,8,9-tetrahydro-5H-benzocyclohepten-6-yl]ethanol (as mixture with 8-methoxy-3a-(4-methoxyphenyl)-3,3a,4,5,6,10b-hexahydro-2H-benzo[6,7]cyclohepta[1,2-b]furan) was introduced under argon, and 142 ml of a 1M DIBAH solution in toluene were added. The reaction mixture was heated under reflux for 2 h and then stirred at RT for 24 h. It was again heated to 80° C., and EtOH was added dropwise (until gas evolution ceased). Cooling to RT was followed by addition of dilute HCl solution and concentration in vacuo. The residue was extracted with ethyl acetate, and the combined org. phases were washed with H₂O and sat. NaCl solution, dried over Na₂SO₄, filtered and dried in vacuo. 6-(2-Hydroxyethyl)-6-(4-hydroxyphenyl)-6,7,8,9-tetrahydro-5H-benzocyclohepten-2-ol was obtained (7.68 g, 99%).

¹H-NMR (DMSO-d₆) δ in ppm: 1.58 (m, 4H, CH₂), 1.80 (m, 1H, CH₂), 2.03 (m, 1H, CH₂), 2.62 (m, 2H, CH₂), 2.80 (m, 1H, CH₂), 3.06 (m, 3H, CH₂), 4.06 (m, 1H, OH), 6.42 (dd, 1H, CH_{Ar}), 6.45 (d, 1H, CH_{Ar}), 6.66 (m, 2H, CH_{Ar}), 6.92 (d, 1H, CH_{Ar}), 7.17 (m, 2H, CH_{Ar}), 9.00 (s, 1H, OH), 9.10 (s, 1H, OH).

### 2-(tert-Butyldimethylsilanyloxy)-6-[2-(tert-butyldimethylsilanyloxy)ethyl]-6-[4-(tert-butyldimethylsilanyloxy)phenyl]-6,7,8,9-tetrahydro-5H-benzocycloheptene

8.89 g of 6-(2-hydroxyethyl)-6-(4-hydroxyphenyl)-6,7,8,9-tetrahydro-5H-benzocyclohepten-2-ol were introduced into 350 ml of DMF, and then 20.3 g of 1H-imidazole and 44.9 g of tert-butyldimethylsilyl chloride were added. The reaction mixture was stirred at RT for 48 h and, while cooling in ice, H₂O was added. After extraction with diethyl ether, the organic phase was washed with water and sat. NaCl solution, dried over MgSO₄, filtered and concentrated. Purification by column chromatography on silica gel and drying in vacuo at 50° C. resulted in 2-(tert-Butyldimethylsilanyloxy)-6-[2-(tert-butyldimethylsilanyloxy)ethyl]-6-[4-(tert-butyldimethylsilanyloxy)phenyl]-6,7,8,9-tetrahydro-5H-benzocycloheptene quantitatively (19.4 g).

¹H-NMR (CDCl₃) δ in ppm: 0.19 (m, 12H, CH₃), 0.99 (m, 18H, tBu), 1.73 (m, 4H, CH₂), 1.97 (m, 2H, CH₂), 2.71 (m, 2H, CH₂), 2.92 (m, 1H, CH₂), 3.17 (m, 1H, CH₂), 3.31 (m, 2H, CH₂), 6.55 (dd, 1H, CH_{Ar}), 6.58 (d, 1H, CH_{Ar}), 6.78 (m, 2H, CH_{Ar}), 6.94 (d, 1H, CH_{Ar}), 7.24 (m, 2H, CH_{Ar}).

### 2-{2-(tert-Butyldimethylsilanyloxy)-6-[4-(tert-butyldimethylsilanyloxy)phenyl]-6,7,8,9-tetrahydro-5H-benzocyclohepten-6-yl}ethanol

19.4 g of 2-(tert-butyldimethylsilanyloxy)-6-[2-(tert-butyldimethylsilanyloxy)ethyl]-6-[4-(tert-butyldimethylsilanyloxy)phenyl]-6,7,8,9-tetrahydro-5H-benzocycloheptene were introduced into 317 ml of acetonitrile, and 22.5 g of cerium trichloride heptahydrate were added. The reaction mixture was heated under reflux for 2 h, and the precipitate resulting after cooling to RT was filtered off. The filtrate was concentrated in vacuo, H₂O was added, and the mixture was extracted with ethyl acetate. The combined org. phases were dried over Na₂SO₄, filtered and concentrated. Purification by column chromatography on silica gel resulted in 2-{2-(tert-butyldimethylsilanyloxy)-6-[4-(tert-butyldimethylsilanyloxy)phenyl]-6,7,8,9-tetrahydro-5H-benzocyclohepten-6-yl}-ethanol (12.2 g, 77%).

### 2-(tert-Butyldimethylsilanyloxy)-6-[4-(tert-butyldimethylsilanyloxy)phenyl]-6-[2-(2-nitrophenylselanyl)ethyl]-6,7,8,9-tetrahydro-5H-benzocycloheptene

7.7 g of 2-{2-(tert-butyldimethylsilanyloxy)-6-[4-(tert-butyldimethylsilanyloxy)phenyl]-6,7,8,9-tetrahydro-5H-benzocyclohepten-6-yl}ethanol were introduced into 50 ml of THF, and 4.98 g of 2-nitrophenyl selenocyanate were added. The mixture was stirred at RT for 5 min and then 5.41 ml of tributylphosphine were added dropwise. After the reaction mixture had been stirred at RT for 1 h it was adsorbed on Isolute and purified by column chromatography on silica gel. 2-(tert-Butyldimethylsilanyloxy)-6-[4-(tert-butyldimethylsilanyloxy)phenyl]-6-[2-(2-nitrophenylselanyl)ethyl]-6,7,8,9-tetrahydro-5H-benzocycloheptene was obtained (8.1 g, 78%).

### 2-(tert-Butyldimethylsilanyloxy)-6-[4-(tert-butyldimethylsilanyloxy)phenyl]-6-vinyl-6,7,8,9-tetrahydro-5H-benzocycloheptene

588 mg of 2-(tert-butyldimethylsilanyloxy)-6-[4-(tert-butyldimethylsilanyloxy)phenyl]-6-[2-(2-nitrophenylselanyl)ethyl]-6,7,8,9-tetrahydro-5H-benzocycloheptene were introduced into 9 ml of dichloromethane at 0° C. 485 mg of calcium carbonate and then 192 mg of m-chloroperbenzoic acid (72% pure) were added. The reaction mixture was warmed to RT and stirred for 2 h. It was filtered through Celite and washed with dichloromethane. The filtrate was washed with H₂O and sat. NaCl solution, dried over Na₂SO₄, filtered and concentrated. The resulting residue was taken up in 20 ml of EtOH, activated carbon was added, and the mixture was stirred at 45° C. for 1 h. It was filtered through Celite, and the filtrate was concentrated and purified by column chromatography on silica gel. 2-(tert-Butyldimethylsilanyloxy)-6-[4-(tert-butyldi methylsilanyloxy)phenyl]-6-vinyl-6,7,8,9-tetrahydro-5H-benzocycloheptene was obtained (259 mg, 63%).

¹H-NMR (CDCl₃) δ in ppm: 0.19 (m, 12H, CH₃), 0.98 (m, 18H, tBu), 1.76 (m, 2H, CH₂), 2.08 (m, 2H, CH₂), 2.74 (m, 2H, CH₂), 3.00 (d, 1H, CH₂), 3.36 (d, 1H, CH₂), 4.80 (dd, 1H, CH_{vinyl}), 5.00 (dd, 1H, CH_{vinyl}), 5.79 (dd, 1H, CH_{vinyl}), 6.54 (m, 2H, CH_{Ar}), 6.75 (m, 2H, CH_{Ar}), 6.88 (d, 1H, CH_{Ar}), 7.23 (m, 2H, CH_{Ar}).

### 6-(4-Hydroxyphenyl)-6-vinyl-6,7,8,9-tetrahydro-5H-benzocyclohepten-2-ol

250 mg of 2-(tert-butyldimethylsilanyloxy)-6-[4-(tert-butyldimethylsilanyloxy)phenyl]-6-vinyl-6,7,8,9-tetrahydro-5H-benzocycloheptene were introduced into 4.2 ml of acetonitrile. 185 mg of 4,7,13,16,21,24-hexaoxa-1,10-diazabicyclo[8.8.8]hexacosane and 523 mg of potassium carbonate were added, and the mixture was stirred at 55° C. for 1 h 45 min. After addition of H₂O, the reaction mixture was concentrated. The residue was extracted with ethyl acetate, and the combined org. phases were washed with sat. NaCl solution, dried over Na₂SO₄, filtered and concentrated. Purification by column chromatography on silica gel resulted in 6-(4-hydroxyphenyl)-6-vinyl-6,7,8,9-tetrahydro-5H-benzocyclohepten-2-ol (123 mg, 89%).

¹H-NMR (DMSO-d₆) δ in ppm: 1.64 (m, 2H, CH₂), 1.95 (m, 1H, CH₂), 2.13 (m, 1H, CH₂), 2.64 (m, 2H, CH₂), 2.89 (d, 1H, CH₂), 3.29 (d, 1H, CH₂), 4.72 (m, 1H, CH_{vinyl}), 4.91 (m, 1H, CH_{vinyl}), 5.76 (m, 1H, CH_{vinyl}), 6.38 (dd, 1H, CH_{Ar}), 6.41 (d, 1H, CH_{Ar}), 6.64 (m, 2H, CH_{Ar}), 6.89 (d, 1H, CH_{Ar}), 7.15 (m, 2H, CH_{Ar}), 8.98 (s, 1H, OH), 9.13 (s, 1H, OH).

### Variant T

### 5-(3-Hydroxypropyl)-2-methoxy-6-(4-methoxyphenyl)-6-methyl-6,7,8,9-tetrahydro-5H-benzocyclohepten-5-ol

### Preparation of the Grignard Solution:

2.51 ml of 3-chloro-1-propanol are dissolved in 30 ml of THF under argon and cooled to −26° C., and 10 ml of 3 molar methylmagnesium chloride solution (in THF) are added dropwise at −26 to −22° C., the mixture is stirred at −24° C. to −21° C. for 25 min, then 1.09 g of magnesium turnings and 10 μl of 1,2-dibromoethane are added, and the reaction mixture is boiled under reflux in an oil bath at a bath temperature of 70-75° C. for 2.5 h. Then a further two portions of 10 μl of 1,2-dibromoethane are added with an interval of 45 min. The reaction flask is flushed with argon and stored under argon.

291 mg of 2-methoxy-6-(4-methoxyphenyl)-6-methyl-6,7,8,9-tetrahydrobenzocyclohepten-5-one are dissolved in 10 ml of THF under argon and cooled to −70° C., and 2.5 ml of Grignard solution are added dropwise, and the mixture is stirred at −70° C. for 30 min, then slowly warmed to RT and stirred at RT for 20 h. The reaction mixture is cooled to +2° C. and a further 3 ml are added, slowly warmed to RT and stirred at RT. The reaction mixture is stirred into 150 ml of sat. NH₄Cl solution, stirred for 30 min and extracted three times with 75 ml of ethyl acetate each time. The organic phase is washed once with sat. NaCl solution, dried over Na₂SO₄, filtered and concentrated: 419 mg of crude product. The crude product is dissolved in ethyl acetate, absorbed on silica gel, dried and put on a 20 g Flashmaster Si column and chromatographed with hexane/ethyl acetate (0 to 50% ethyl acetate): 136 mg of 5-(3-hydroxypropyl)-2-methoxy-6-(4-methoxyphenyl)-6-methyl-6,7,8,9-tetrahydro-5H-benzocyclohepten-5-ol: C₂₃H₃₀O₄, MH⁺-18: 353, MH⁺=371 m/e.

### Variant U

### 5-(Spiro-2-tetrahydrofuran)-2-methoxy-6-(4-methoxyphenyl)-6-methyl-6,7,8,9-tetrahydro-5H-benzocycloheptene

413 mg of 5-(3-hydroxypropyl)-2-methoxy-6-(4-methoxyphenyl)-6-methyl-6,7,8,9-tetrahydro-5H-benzocyclohepten-5-ol are dissolved in 10 ml of dichloromethane under argon, 0.62 ml of triethylamine and 323 mg of p-toluenesulphonyl chloride are added, and then 22 mg of DMAP are added and the reaction mixture is stirred at RT for 18 h. The reaction mixture is diluted with dichloromethane, washed once with 50 ml of saturated NaHCO₃ solution and once with saturated NaCl solution, dried over Na₂SO₄, filtered and concentrated: 502 mg. The crude product is dissolved in a little dichloromethane, absorbed on silica gel, dried, put on a 20 g Flashmaster Si column and chromatographed with hexane/ethyl acetate (0 to 30% ethyl acetate): 309 mg of 5-(spiro-2-tetrahydrofuran)-2-methoxy-6-(4-methoxyphenyl)-6-methyl-6,7,8,9-tetrahydro-5H-benzocycloheptene: C₂₃H₂₈O₃, MH⁺ 353 m/e.

### Variant V

### 6-(4-Hydroxyphenyl)-5-vinyl-6,7,8,9-tetrahydro-5H-benzocyclohepten-2-ol

### 5-(2-Benzyloxyethyl)-2-methoxy-5,7,8,9-tetrahydrobenzocyclohepten-6-one

10.0 g of 2-methoxy-5,7,8,9-tetrahydrobenzocyclohepten-6-one were dissolved in 180 ml of THF at 0° C. Addition of 6.02 g of potassium hydride was followed by warming to RT and stirring at RT for 2 h. The reaction mixture was again cooled to 0° C., and 8.30 ml of (2-bromoethoxymethyl)benzene were added dropwise. The mixture was warmed to RT and stirred at RT for 2 h. After addition of H₂O, the reaction mixture was concentrated in vacuo, and the resulting residue was extracted with ethyl acetate. The combined organic phases were dried over Na₂SO₄, filtered, concentrated and dried in vacuo. Purification by column chromatography on silica gel resulted in 5-(2-benzyloxyethyl)-2-methoxy-5,7,8,9-tetrahydrobenzocyclohepten-6-one: C₂₁H₂₄O₃: MH⁺=325 m/e (6.5 g, as approx. 30% mixture with 2-methoxy-5,7,8,9-tetrahydrobenzocyclohepten-6-one). This mixture was directly reacted further.

### 5-(2-Benzyloxyethyl)-2-methoxy-8,9-dihydro-7H-benzocyclohepten-6-yl nonafluorobutane-1-sulphonate

6.50 g of 5-(2-benzyloxyethyl)-2-methoxy-5,7,8,9-tetrahydrobenzocyclohepten-6-one (approx. 30% mixture with 2-methoxy-5,7,8,9-tetrahydrobenzocyclohepten-6-one) were dissolved in 77 ml of toluene at 0° C. Addition of 18.2 ml of DBU was followed by stirring at 0° C. for 15 min, and then 20.2 ml of nonaflyl fluoride were added. After stirring at RT for 70 min, the reaction mixture was added to 1000 ml of NH₄Cl solution. After extraction with methyl tert-butyl ether, the organic phase was washed with water and sat. NaCl solution, dried over Na₂SO₄, filtered, concentrated and dried in vacuo. 5-(2-Benzyloxyethyl)-2-methoxy-8,9-dihydro-7H-benzocyclohepten-6-yl nonafluorobutane-1-sulphonate was obtained: C₂₅H₂₃F₉O₅S: MH⁺=607 m/e (19.2 g, as mixture with 2-methoxy-8,9-dihydro-7H-benzocyclohepten-6-yl nonafluorobutane-1-sulphonate. This mixture was directly reacted further.

### 9-(2-Benzyloxyethyl)-3-methoxy-8-(4-methoxyphenyl)-6,7-dihydro-5H-benzocycloheptene

4.70 g of 5-(2-benzyloxyethyl)-2-methoxy-8,9-dihydro-7H-benzocyclohepten-6-yl nonafluorobutane-1-sulphonate (as mixture with 2-methoxy-8,9-dihydro-7H-benzocyclohepten-6-yl nonafluorobutane-1-sulphonate) and 1.67 g of p-methoxyphenylboronic acid were introduced into a mixture of 14.5 ml of toluene and 14.5 ml of ethanol under argon. 537 mg of Pd(PPh₃)₄, 2.77 g of Na₂CO₃ and 6.3 ml of H₂O were added, and the mixture was then heated in a microwave reactor at 120° C. for 1 h. The reaction mixture was filtered through Celite and washed with ethyl acetate. The phases were separated, and the aqueous phase was extracted with ethyl acetate. The combined org. phases were washed with sat. NaCl solution, dried over Na₂SO₄, filtered and concentrated. Purification by column chromatography on silica gel resulted in 9-(2-benzyloxyethyl)-3-methoxy-8-(4-methoxyphenyl)-6,7-dihydro-5H-benzocycloheptene (6.5 g, as mixture with 3-methoxy-8-(4-methoxyphenyl)-6,7-dihydro-5H-benzocycloheptene). This mixture was directly reacted further.

### 2-[2-Methoxy-6-(4-methoxyphenyl)-6,7,8,9-tetrahydro-5H-benzocyclohepten-5-yl]-ethanol

2.84 g of 9-(2-benzyloxyethyl)-3-methoxy-8-(4-methoxyphenyl)-6,7-dihydro-5H-benzocycloheptene (as mixture with 3-methoxy-8-(4-methoxyphenyl)-6,7-dihydro-5H-benzocycloheptene) were dissolved in 30 ml of methanol. Addition of 84 mg of Pd(OH)₂ was followed by stirring in an autoclave under an H₂ pressure of 200 bar at 80° C. for 7 h. The reaction mixture was filtered through Celite and concentrated. Purification by column chromatography on silica gel resulted in 2-[2-methoxy-6-(4-methoxyphenyl)-6,7,8,9-tetrahydro-5H-benzocyclohepten-5-yl]ethanol: C₂₁H₂₆O₃: MH⁺=327 m/e (605 mg, 27%).

¹H-NMR (CDCl₃) δ in ppm: 1.58 (m, 1H, CH₂), 1.75 (m, 1H, CH₂), 2.06 (m, 4H, CH₂), 2.77 (m, 1H, CH), 3.01 (m, 3H, CH/CH₂), 3.38 (m, 2H, CH₂), 3.81 (s, 6H, OCH₃), 6.66 (dd, 1H, CH_{Ar}), 6.71 (d, 1H, CH_{Ar}), 6.85 (m, 2H, CH_{Ar}), 7.02 (d, 1H, CH_{Ar}), 7.13 (m, 2H, CH_{Ar}).

### 5-(2-Hydroxyethyl)-6-(4-hydroxyphenyl)-6,7,8,9-tetrahydro-5H-benzocyclohepten-2-ol

517 mg of 2-[2-methoxy-6-(4-methoxyphenyl)-6,7,8,9-tetrahydro-5H-benzocyclohepten-5-yl]ethanol were introduced under argon, and 28 ml of a 1.5M DIBAH solution in toluene were added. The reaction mixture was heated under reflux for 23 h and then, at 80° C., EtOH was added dropwise (until gas evolution ceased). Cooling to RT was followed by addition of dilute HCl solution and extraction with toluene. The combined org. phases were washed with H₂O and sat. NaCl solution, dried over Na₂SO₄, filtered and concentrated. Purification by column chromatography on silica gel resulted in 5-(2-hydroxyethyl)-6-(4-hydroxyphenyl)-6,7,8,9-tetrahydro-5H-benzocyclohepten-2-ol quantitatively: C₁₉H₂₂O₃: MH⁺=299 m/e.

### 2-(tert-Butyldimethylsilanyloxy)-5-[2-(tert-butyldimethylsilanyloxy)ethyl]-6-[4-(tert-butyldimethylsilanyloxy)phenyl]-6,7,8,9-tetrahydro-5H-benzocycloheptene

524 mg of 5-(2-hydroxyethyl)-6-(4-hydroxyphenyl)-6,7,8,9-tetrahydro-5H-benzocyclohepten-2-ol were introduced into 27 ml of DMF and then 1.79 g of 1H-imidazole and 3.97 g of tert-butyldimethylsilyl chloride were added. The reaction mixture was stirred at RT for 17 h and, while cooling in ice, H₂O was added. After extraction with diethyl ether, the organic phase was washed with water and sat. NaCl solution, dried over MgSO₄, filtered and concentrated. Purification by column chromatography on silica gel resulted in 2-(tert-butyldimethylsilanyloxy)-5-[2-(tert-butyldimethylsilanyloxy)ethyl]-6-[4-(tert-butyldimethylsilanyloxy)phenyl]-6,7,8,9-tetrahydro-5H-benzocycloheptene (1.11 g, 99%).

### 2-{2-(tert-Butyldimethylsilanyloxy)-6-[4-(tert-butyldimethylsilanyloxy)phenyl]-6,7,8,9-tetrahydro-5H-benzocyclohepten-5-yl}ethanol

1.11 g of 2-(tert-butyldimethylsilanyloxy)-5-[2-(tert-butyldimethylsilanyloxy)ethyl]-6-[4-(tert-butyldimethylsilanyloxy)phenyl]-6,7,8,9-tetrahydro-5H-benzocycloheptene were introduced into 50 ml of acetonitrile, and 1.29 g of cerium trichloride heptahydrate were added. The reaction mixture was heated under reflux for 7.5 h and then stirred at RT for 15.5 h. Addition of H₂O was followed by extraction with ethyl acetate. The combined org. phases were dried over Na₂SO₄, filtered and concentrated. Purification by column chromatography on silica gel resulted in 2-{2-(tert-butyldimethylsilanyloxy)-6-[4-(tert-butyldimethylsilanyloxy)phenyl]-6,7,8,9-tetrahydro-5H-benzocyclohepten-5-yl}ethanol (474 mg, 52%).

¹H-NMR (DMSO-d₆) δ in ppm: 0.18 (m, 12H, CH₃), 0.95 (m, 18H, tBu), 1.50 (m, 2H, CH₂), 1.80 (m, 2H, CH₂), 2.07 (m, 2H, CH₂), 2.72 (m, 1H, CH), 3.00 (m, 5H, CH/CH₂), 4.21 (m, 1H, OH), 6.54 (dd, 1H, CH_{Ar}), 6.61 (d, 1H, CH_{Ar}), 6.74 (m, 2H, CH_{Ar}), 6.90 (m, 1H, CH_{Ar}), 7.03 (m, 2H, CH_{Ar}).

### 2-(tert-Butyldimethylsilanyloxy)-6-[4-(tert-butyldimethylsilanyloxy)phenyl]-5-[2-(2-nitrophenylselanyl)ethyl]-6,7,8,9-tetrahydro-5H-benzocycloheptene

245 mg of 2-{2-(tert-butyldimethylsilanyloxy)-6-[4-(tert-butyldimethylsilanyloxy)-phenyl]-6,7,8,9-tetrahydro-5H-benzocyclohepten-5-yl}ethanol were introduced into 1.6 ml of THF and 158 mg of 2-nitrophenyl selenocyanate were added. The mixture was stirred at RT for 5 min and then 0.17 ml of tributylphosphine was added dropwise. After stirring at RT for 75 min, the reaction mixture was adsorbed on Isolute and purified by column chromatography on silica gel. 2-(tert-Butyldimethyl-silanyloxy)-6-[4-(tert-butyldimethylsilanyloxy)phenyl]-5-[2-(2-nitrophenylselanyl)ethyl]-6,7,8,9-tetrahydro-5H-benzocycloheptene was obtained (275 mg, 81%).

### 2-(tert-Butyldimethylsilanyloxy)-6-[4-(tert-butyldimethylsilanyloxy)phenyl]-5-vinyl-6,7,8,9-tetrahydro-5H-benzocycloheptene

275 mg of 2-(tert-butyldimethylsilanyloxy)-6-[4-(tert-butyldimethylsilanyloxy)phenyl]-5-[2-(2-nitrophenylselanyl)ethyl]-6,7,8,9-tetrahydro-5H-benzocycloheptene were introduced into 4.4 ml of dichloromethane at 0° C. 227 mg of calcium carbonate and then 90 mg of m-chloroperbenzoic acid (72% pure) were added. The reaction mixture was warmed to RT and stirred for 1.5 h. It was filtered through Celite and washed with dichloromethane. The filtrate was washed with sat. NaCl solution, dried over Na₂SO₄, filtered and concentrated. The resulting residue was taken up in 12 ml of EtOH, mixed with activated carbon and stirred at 45° C. for 1 h. It was filtered through Celite, and the filtrate was concentrated and purified by column chromatography on silica gel. 2-(tert-Butyldimethylsilanyloxy)-6-[4-(tert-butyldimethylsilanyloxy)phenyl]-5-vinyl-6,7,8,9-tetrahydro-5H-benzocycloheptene was obtained: C₃₁H₄₈O₂Si₂: MH⁺=509 m/e (152 mg, 79%).

### 6-(4-Hydroxyphenyl)-5-vinyl-6,7,8,9-tetrahydro-5H-benzocyclohepten-2-ol

150 mg of 2-(tert-butyldimethylsilanyloxy)-6-[4-(tert-butyldimethylsilanyloxy)phenyl]-5-vinyl-6,7,8,9-tetrahydro-5H-benzocycloheptene were introduced into 2.5 ml of acetonitrile. 111 mg of 4,7,13,16,21,24-hexaoxa-1,10-diazabicyclo[8.8.8]hexacosane and 313 mg of potassium carbonate were added, and the mixture was stirred at 55° C. for 45 min. Addition of H₂O was followed by extraction with ethyl acetate. The combined org. phases were washed with sat. NaCl solution, dried over Na₂SO₄, filtered and concentrated. Purification by column chromatography on silica gel resulted in 6-(4-hydroxyphenyl)-5-vinyl-6,7,8,9-tetrahydro-5H-benzocyclohepten-2-ol: C₁₉H₂₀O₂: MH⁺=281 m/e (65 mg, 79%).

¹H-NMR (DMSO-d₆) δ in ppm: 1.41 (m, 1H, CH₂), 1.82 (m, 1H, CH₂), 1.99 (m, 1H, CH₂), 2.14 (m, 1H, CH₂), 2.56 (m, 1H, CH), 2.86 (m, 2H, CH₂), 3.47 (m, 1H, CH), 4.51 (m, 1H, CH_{vinyl}), 4.98 (m, 1H, CH_{vinyl}), 6.12 (m, 1H, CH_{vinyl}), 6.49 (dd, 1H, CH_{Ar}), 6.54 (d, 1H, CH_{Ar}), 6.68 (m, 2H, CH_{Ar}), 6.91 (d, 1H, CH_{Ar}), 7.02 (m, 2H, CH_{Ar}), 9.15 (m, 2H, OH).

| | Preparation | |
|---|---|---|
| Example | (variant) | Precursors |
| 1 | A + B | 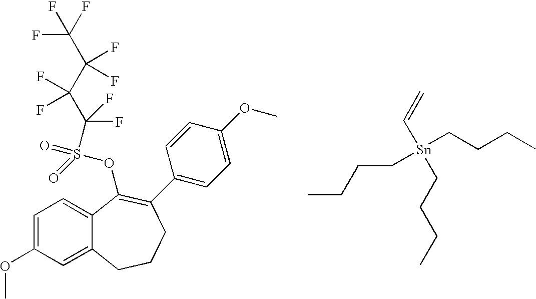 |
| 2 | C | 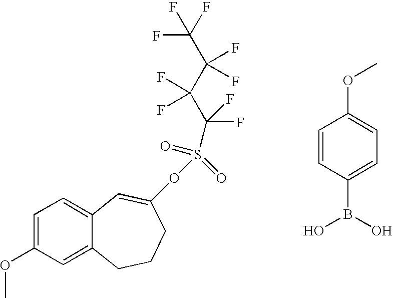 |
| 3 | D | 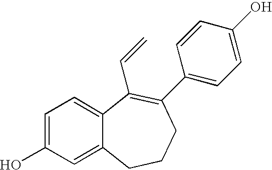 |
| 3 | G + H | 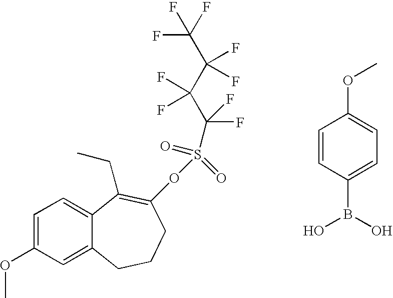 |
| 3 | A + H | 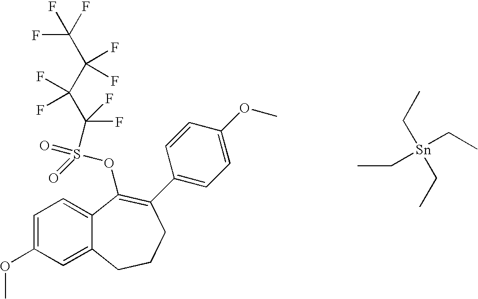 |
| 4 | F + H | 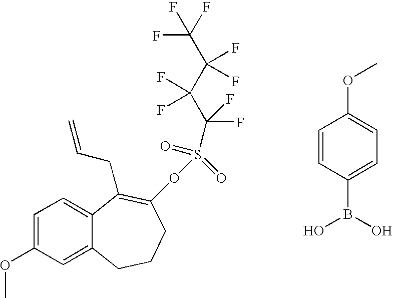 |
| 5 | E | 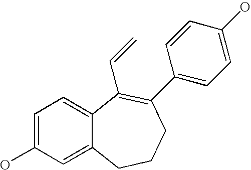 |
| 6 | I | 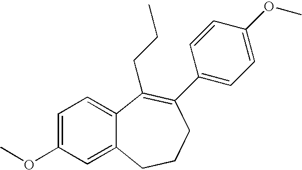 |
| 6 | D | 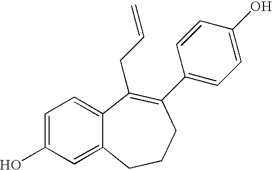 |
| 7 | E | 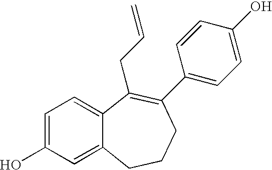 |
| 8 | G + B | 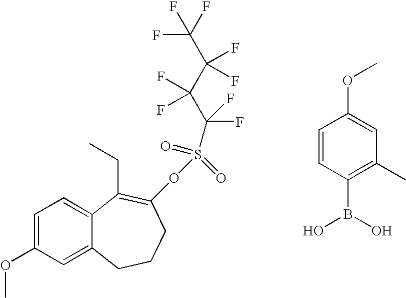 |
| 9 | J + K + B | 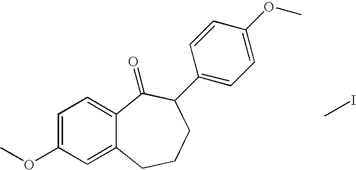 |
| 10 | J + K + B | 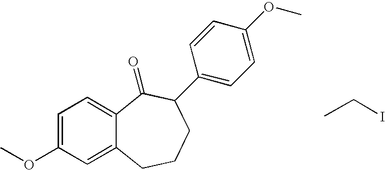 |
| 11 | J + K + B | 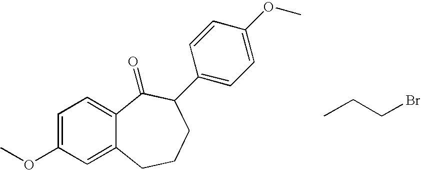 |
| 12 | J + K + B | 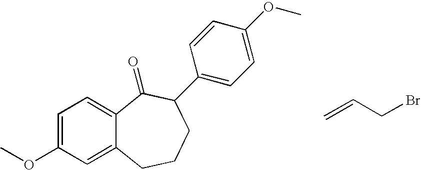 |
| 13 | A + I | 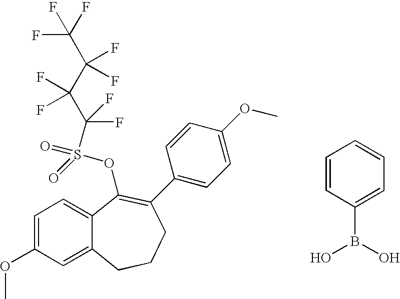 |
| 14 | A + I | 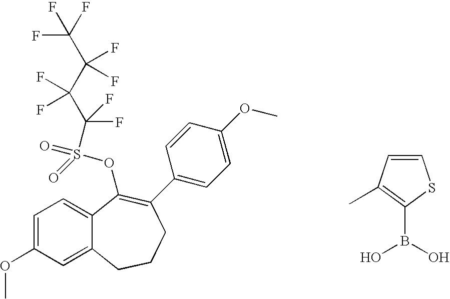 |
| 15 | F + I | 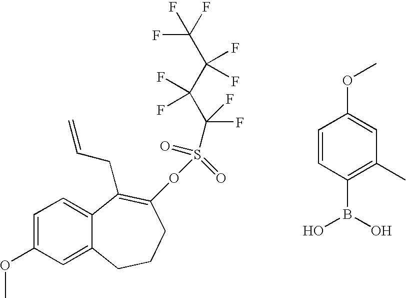 |
| 16 | F + I | 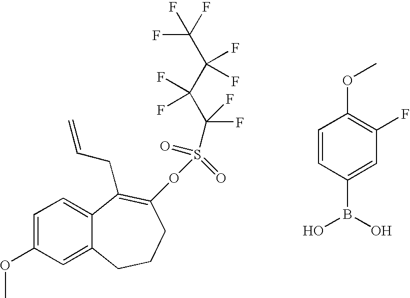 |
| 17 | F + I | 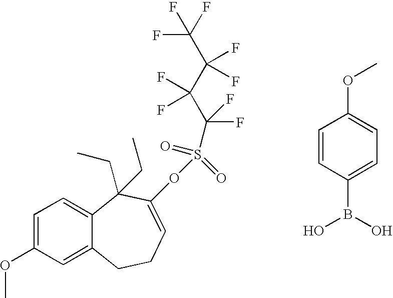 |
| 18 | C | 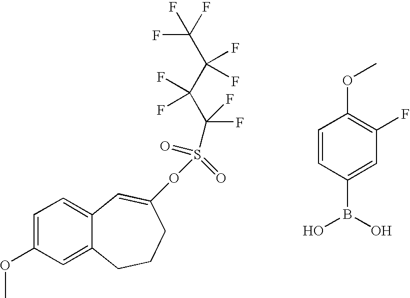 |
| 19 | D | 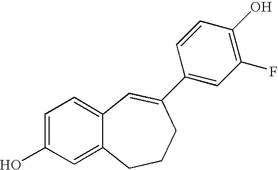 |
| 20 | G + D | 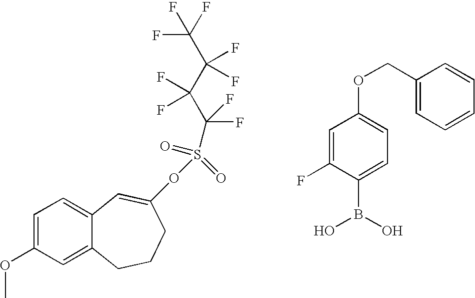 |
| 21 | G + D + B | 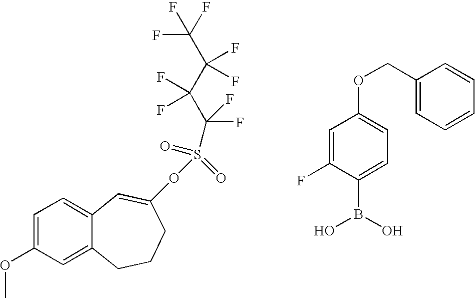 |
| 22 | L + M + R | 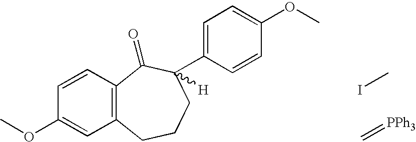 |
| 23 | L + N + O + P + Q | 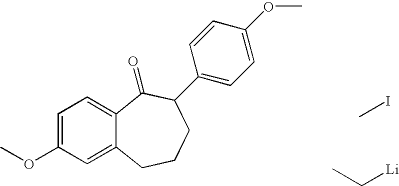 |
| 24 | L + N + O + P + Q | 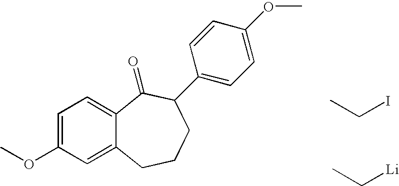 |
| 25 | R | 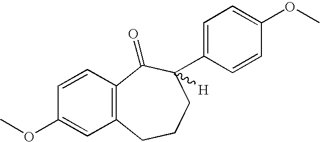 |
| 26 | L + R | 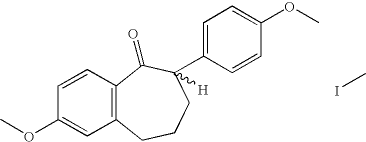 |
| 27 | L + T + U + I | 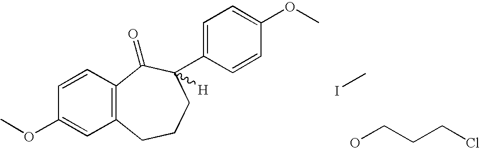 |
| 28 | S | 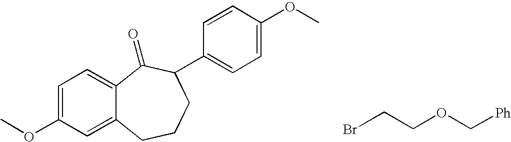 |
| 29 | L + N + O + R | 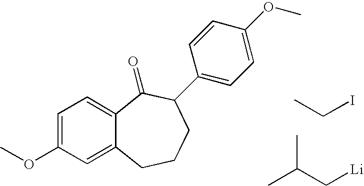 |
| 30 | L + N + R | 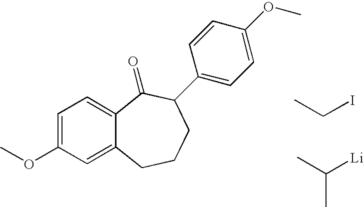 |
| 31 | L + N + O + P + R | 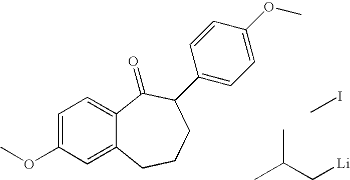 |
| 32 | L + M + R | 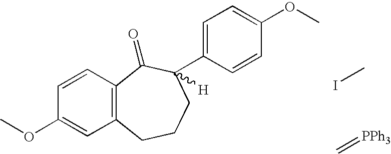 |
| 33 | A + P + R | 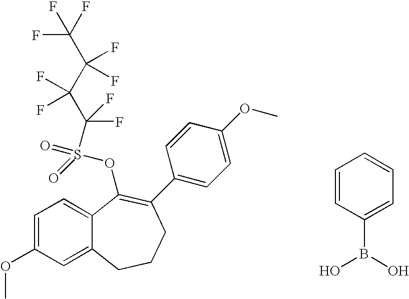 |
| 34 | V | 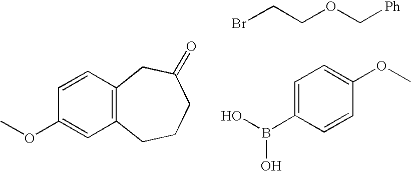 |

| | | Molecular |
|---|---|---|
| | Structural | mass |
| Example | formula | found |
| 1 | 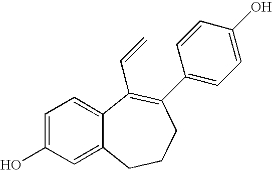 | 278 |
| 2 | 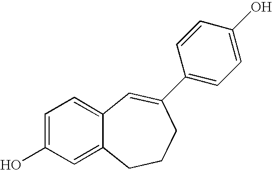 | 252 |
| 3 | 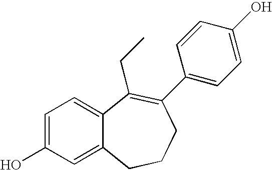 | 280 |
| 3 | 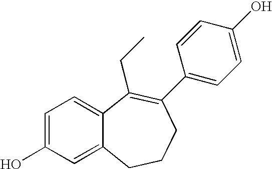 | 280 |
| 3 | 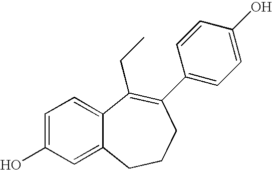 | 280 |
| 4 | 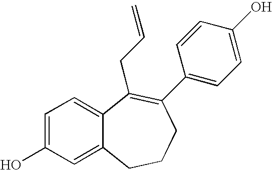 | 292 |
| 5 | 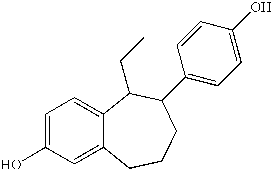 | 282 |
| 6 | 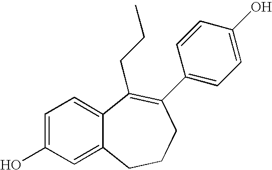 | 294 |
| 6 | 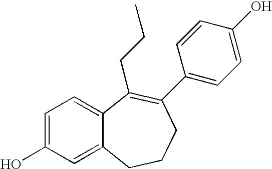 | 294 |
| 7 | 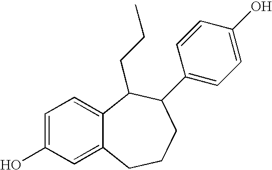 | 296 |
| 8 | 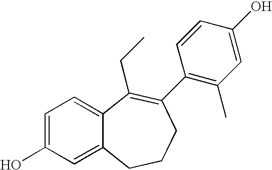 | 294 |
| 9 | 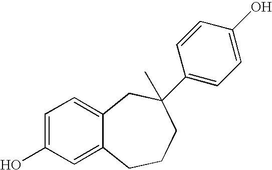 | 268 |
| 10 | 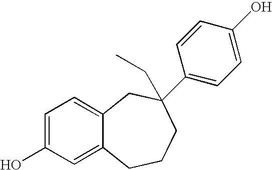 | 282 |
| 11 | 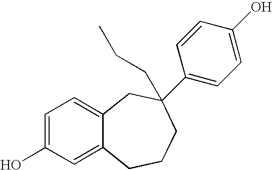 | 296 |
| 12 | 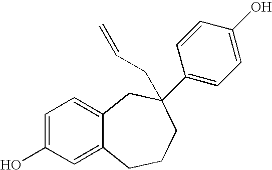 | 294 |
| 13 | 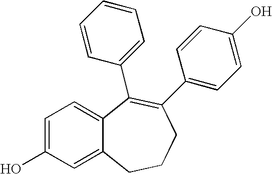 | 328 |
| 14 | 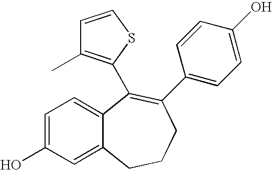 | 348 |
| 15 | 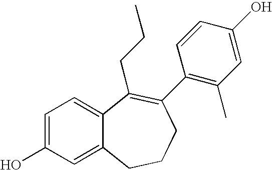 | 308 |
| 16 | 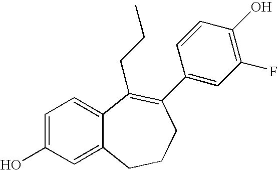 | 312 |
| 17 | 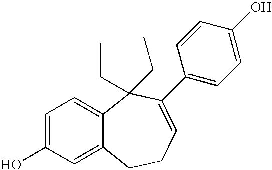 | 308 |
| 18 | 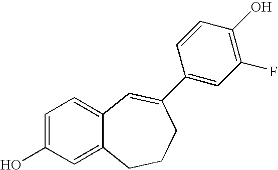 | 270 |
| 19 | 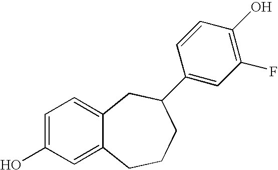 | 272 |
| 20 | 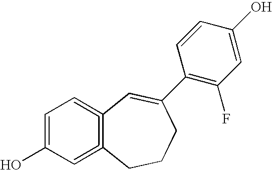 | 284 |
| 21 | 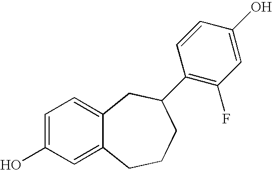 | 272 |
| 22 | 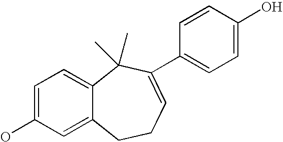 | 281 |
| 23 | 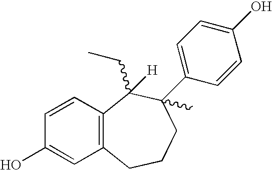 | 296 |
| 24 | 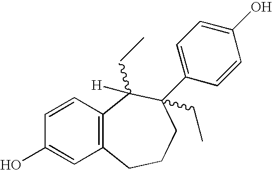 | 310 |
| 25 | 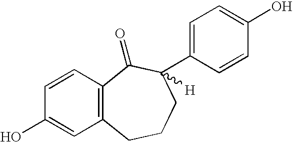 | 268 |
| 26 | 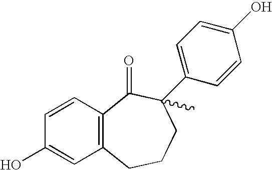 | 282 |
| 27 | 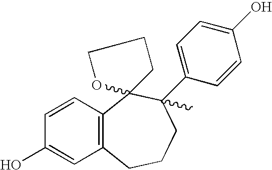 | 324 |
| 28 | 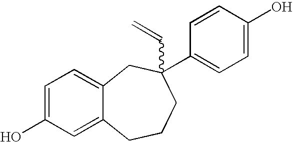 | 280 |
| 29 | 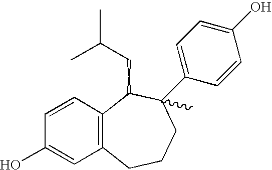 | 322 |
| 30 | 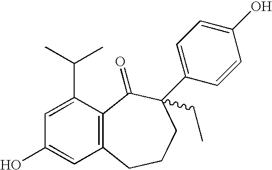 | 338 |
| 31 | 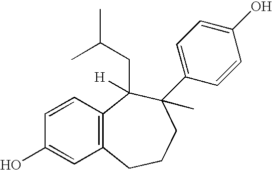 | 324 |
| 32 | 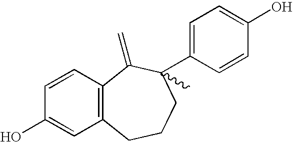 | 281 |
| 33 | 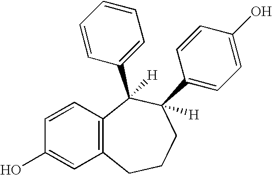 | 330 |
| 34 | 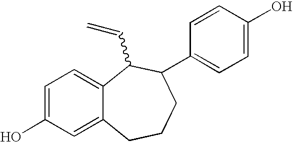 | 280 |

TABLE 1

| | | | | | Rat | Rat | |
|---|---|---|---|---|---|---|---|
| | | hER α | hER β | ERβ/ | uterus | prost. | prost. ER/ |
| Estrogen | Structure | RBA* | RBA* | Erα | ER(RBA) | ER(RBA) | uterus ER |
| Estradiol | 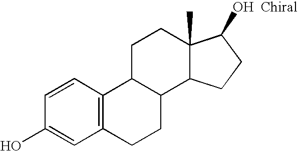 | 100 | 100 | 1 | 100 | 100 | 1 |
| Estrone | 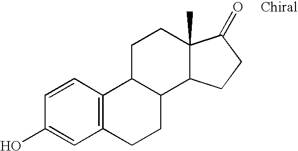 | 60 | 37 | 0.6 | 3 | 2 | 0.8 |
| 17α-Estradiol | 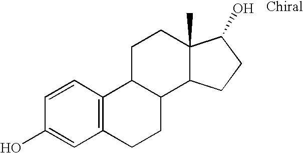 | 58 | 11 | 0.2 | 2.4 | 1.3 | 0.5 |
| Estriol | 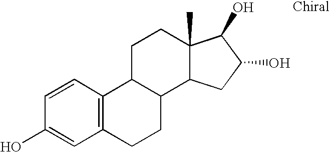 | 14 | 21 | 1.5 | 4 | 20 | 5 |
| 5-Androstene-diol | 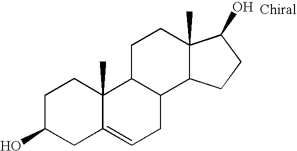 | 6 | 17 | 3 | 0.1 | 5 | 50 |
| Genistein | 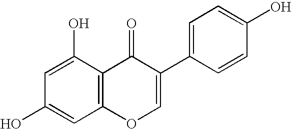 | 5 | 36 | 7 | 0.1 | 10 | 100 |
| Coumestrol | 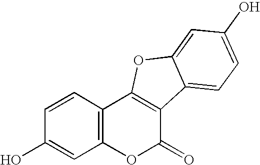 | 94 | 185 | 2 | 1.3 | 24 | 18 |

*quoted from: Kuiper et al. (1996), Endocrinology 138: 863-870

TABLE 2

| | Rat Uterus | Rat Prostate | Prostate ER/ |
|---|---|---|---|
| Structure | ER(RBA) | ER(RBA) | Uterus ER |
| 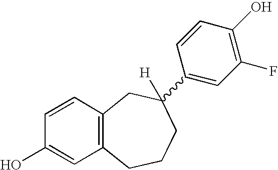 | 1.5 | 1.3 | 0.9 |
| 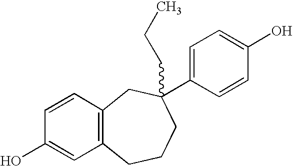 | 2 | 0.7 | 0.4 |
| 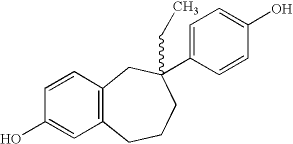 | 3 | 5 | 1.7 |
| 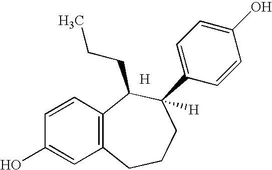 | 30 | 25 | 0.8 |
| 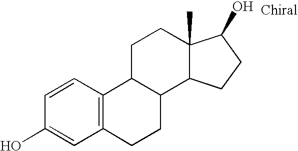 | 100 | 100 | 1 |

TABLE 3

| | GAL4 | GAL4- |
|---|---|---|
| | assay, | assay, |
| | Potency | Potency |
| | at ERa | at ERb |
| MOLSTRUCTURE | (% of E2) | (% of E2) |
| 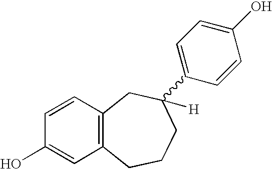 | <0.1 | 8.0 |
| 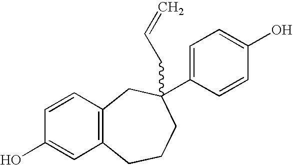 | <0.1 | 0.5 |
| 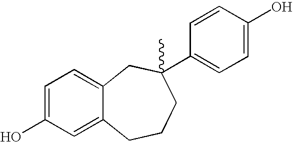 | 0.8 | 19.0 |
| 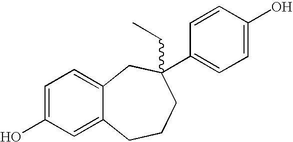 | 3.0 | 23.0 |
| 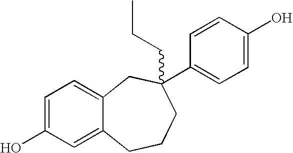 | 0.8 | 2.0 |
| 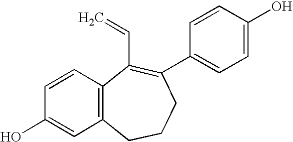 | 0.6 | 5.0 |
| 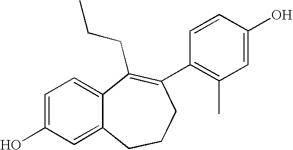 | 43.0 | 78.0 |
| 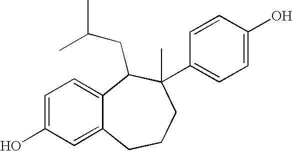 | 3.0 | 10.0 |
| 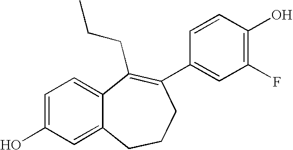 | 39.0 | 48.0 |
| 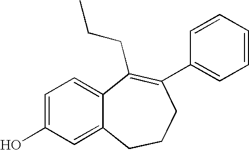 | 6.0 | 5.0 |
| 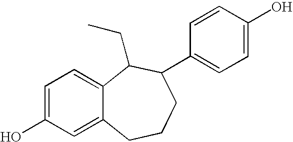 | 15.0 | 55.0 |
| 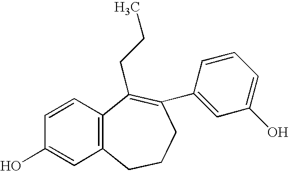 | 0.3 | 16.0 |
| 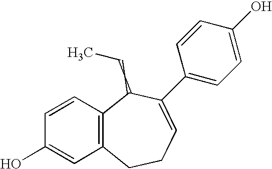 | <0.1 | 0.7 |
| 17b-Estradiol | 100.0 | 100.0 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07075893.3, filed Oct. 11, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula (I) 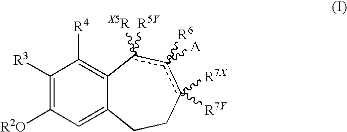
in which
A is 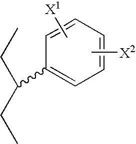
in which
X¹ means one or more groups on the phenyl ring and represents independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² means one or more groups on the phenyl ring and represents independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H or an F atom;
R⁴ is an H atom, a (C₁-C₄)alkyl, (C₁-C₄)alkenyl, (C₁-C₄)alkynyl, perfluoro(C₁-C₄)alkyl, silyl(C₁-C₄)alkyl radical, a halogen atom or a nitrile radical;
R^{5X} and R^{5Y} mean independently of one another an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group, or
R^{5X} and R^{5Y} together are an O atom, an S atom, a ═CHZ in which Z means an H atom, a (C₁-C₄)alkyl, (C₁-C₄)alkenyl radical, or
R^{5X} and R^{5Y} together are a —(C₂-C₄)alkanediyl, (C₂-C₆)alkenediyl radical or —OCH₂— or
R^{5Y} and R⁶ together represent a bond, a —(C₁-C₄)alkanediyl or a (C₂-C₆)alkenediyl radical, and R^{5X} has the meaning given above;
R⁶ means an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical, or
R⁶ and R^{5Y} together represent a bond, a —(C₁-C₄)alkanediyl or a (C₂-C₆)alkenediyl radical, and R^{5X} has the meaning given above, or
R⁶ and R^{7Y} together represent a bond, a —(C₁-C₄)alkanediyl or a (C₂-C₆)alkenediyl radical, and R^{7X} has the meaning given under;
R^{7X} and R^{7Y} represent independently of one another an H atom, a (C₁-C₄)-alkyl, a (C₁-C₄)alkenyl radical, a (C₁-C₄)alkynyl-, or
R^{7X} and R^{7Y} together represent a —(C₂-C₄)alkanediyl, or (C₂-C₆)alkenediyl radical, or
R^{7Y} and R⁶ together represent a bond, a —(C₁-C₄)alkanediyl or a (C₂-C₆)alkenediyl radical, and R^{7X} has the meaning given above.

2. Compounds of the general formula (I) according to claim 1 in which
A is 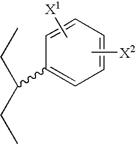
in which
X¹ is one or more groups on the phenyl ring and is independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² is one or more groups on the phenyl ring and is independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H atom or an F atom;
R⁴ is an H atom, a (C₁-C₄)alkyl, (C₁-C₄)alkenyl, (C₁-C₄)alkynyl, perfluoro(C₁-C₄)alkyl, silyl(C₁-C₄)alkyl radical, a halogen atom or a nitrile radical;
R^{5X} is an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group;
R^{5Y} and R⁶ together are a bond;
R^{7X} and R^{7Y} are independently of one another an H atom, a (C₁-C₄)-alkyl, a (C₁-C₄)alkenyl radical, a (C₁-C₄)alkynyl-, or
R^{7X} and R^{7Y} together are —(C₂-C₄)alkanediyl, or (C₂-C₆)alkenediyl radical.

3. Compounds of the general formula (I) according to claim 2
in which R^{5X} is an H atom, and
R^{7X} and R^{7Y} are independently of one another an H atom, a (C₁-C₄)-alkyl, a (C₁-C₄)alkenyl radical, a (C₁-C₄)alkynyl-.

4. Compounds of the general formula (I) according to claim 2
in which R^{5X}, and R^{7Y} are an H atom, and
R^{7X} is an H atom, a (C₁-C₄)-alkyl, a (C₁-C₄)alkenyl radical, or a (C₁-C₄)alkynyl-.

5. Compounds of the general formula (I) according to claim 2
in which R^{5X} is an H atom, and
R^{7X} and R^{7Y} together are a —CH₂—CH═CH—CH₂— or (—CH₂—CH₂—CH₂—CH₂—).

6. Compounds of the general formula (I) according to claim 2
in which R^{5X} is a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group, and
R^{7X} and R^{7Y} are independently of one another an H atom, a (C₁-C₄)-alkyl, a (C₁-C₄)alkenyl radical, or a (C₁-C₄)alkynyl-.

7. Compounds of the general formula (I) according to claim 1
in which
A is 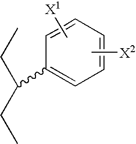
in which
X¹ is one or more groups on the phenyl ring and is independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² is one or more groups on the phenyl ring and is independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H atom or an F atom;
R⁴ is an H atom, a (C₁-C₄)alkyl, (C₁-C₄)alkenyl, (C₁-C₄)alkynyl, perfluoro(C₁-C₄)alkyl, silyl(C₁-C₄)alkyl radical, a halogen atom or a nitrile radical;
R^{5X} and R^{5Y} are independently of one another an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group, or
R^{5X} and R^{5Y} together are an O atom, an S atom, a ═CHZ in which Z is an H atom, a (C₁-C₄)alkyl, (C₁-C₄)alkenyl radical, or
R^{5X} and R^{5Y} together are a —(C₂-C₄)alkanediyl, (C₂-C₆)alkenediyl radical or —OCH₂—;
R⁶ and R^{7Y} together are a bond;
R^{7X} is an H atom, a (C₁-C₄)-alkyl, a (C₁-C₄)alkenyl radical, a (C₁-C₄)alkynyl-.

8. Compounds of the general formula (I) according to claim 7 in which
R^{5X} and R^{5Y} are independently of one another an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group;
R^{7X} is an H atom.

9. Compounds of the general formula (I) according to claim 7
in which R^{5X}, and R^{7Y} are an H atom.

10. Compounds of the general formula (I) according to claim 7
in which R^{5X}, R^{5Y}, and R^{7X} are an H atom.

11. Compounds of the general formula (I) according to claim 7
in which R^{5X} is an H atom, and
R^{7X} and R^{7Y} are independently of one another a (C₁-C₄)-alkyl, a (C₁-C₄)alkenyl radical or a (C₁-C₄)alkynyl-.

12. Compounds of the general formula (I) according to claim 7
in which R^{5X}, and R^{7Y} are an H atom, and
R^{7X} is an H atom, a (C₁-C₄)-alkyl, a (C₁-C₄)alkenyl radical, or a (C₁-C₄)alkynyl-.

13. Compounds of the general formula (I) according to claim 7
in which R^{5X} is an H atom, and
R^{7X} and R^{7Y} together are a —(C₂-C₄)alkanediyl, or (C₂-C₆)alkenediyl radical.

14. Compounds of the general formula (I) according to claim 7
in which R^{5X} is a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl a CN group, and
R^{7X} and R^{7Y} are independently of one another an H atom, a (C₁-C₄)-alkyl, a (C₁-C₄)alkenyl radical, or a (C₁-C₄)alkynyl-.

15. Compounds of the general formula (I) according to claim 1
in which 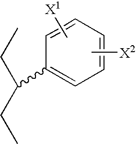
A is
in which
X¹ is one or more groups on the phenyl ring and is independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² is one or more groups on the phenyl ring and is independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H atom or an F atom;
R⁴ is an H atom, a (C₁-C₄)alkyl, (C₁-C₄)alkenyl, (C₁-C₄)alkynyl, perfluoro(C₁-C₄)alkyl, silyl(C₁-C₄)alkyl radical, a halogen atom or a nitrile radical;
R^{5X} and R^{5Y} are independently of one another an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group, or
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7X} and R^{7Y} are independently of one another an H atom, a (C₁-C₄)-alkyl, a (C₁-C₄)alkenyl radical, a (C₁-C₄)alkynyl-.

16. Compounds of the general formula (I) according to claim 15 in which
R^{5X} is an H atom;
R^{5Y} is an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group;
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7X} and R^{7Y} are independently of one another an H atom, a (C₁-C₄)-alkyl, a (C₁-C₄)alkenyl radical, a (C₁-C₄)alkynyl-.

17. Compounds of the general formula (I) according to claim 15 in which
R^{5X} and R^{5Y} are an H atom;
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7X} and R^{7Y} are independently of one another an H atom, a (C₁-C₄)-alkyl, a (C₁-C₄)alkenyl radical, a (C₁-C₄)alkynyl-.

18. Compounds of the general formula (I) according to claim 15 in which
R^{5X} and R^{5Y} are independently of one another an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group;
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7X} and R^{7Y} are an H atom.

19. Compounds of the general formula (I) according to claim 15 in which
R^{5X} and R^{5Y} are independently of one another an H atom, a (C₁-C₄)alkyl, (C₁-C₄)alkenyl, (C₁-C₄)alkynyl, perfluoro(C₁-C₄)alkyl, perfluoro(C₁-C₄)alkenyl, (C₁-C₄)alkyl-O, aryl, heteroaryl or a CN group;
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7X} is an H atom;
R^{7Y} is an H atom, a (C₁-C₄)-alkyl, a (C₁-C₄)alkenyl, or a (C₁-C₄)alkynyl radical.

20. Compounds of the general formula (I) according to claim 15 in which
R⁶ is an H atom.

21. Compounds of the general formula (I) according to claim 1,
in which
A is 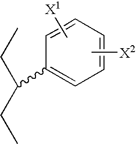
in which
X¹ is one or more groups on the phenyl ring and is independently of one another a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² is one or more groups on the phenyl ring and is independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN;
R² is an H atom, a (C₁-C₄)alkyl radical, a (C₃-C₆)cycloalkyl radical or a (C₁-C₁₄)acyl radical;
R³ is an H atom or an F atom;
R⁴ is an H atom, a (C₁-C₄)alkyl, (C₁-C₄)alkenyl, (C₁-C₄)alkynyl, perfluoro(C₁-C₄)alkyl, silyl(C₁-C₄)alkyl radical, a halogen atom or a nitrile radical;
R^{5X} and R^{5Y} together are an ═O or ═CHZ in which Z is an H atom, a (C₁-C₄)alkyl, or (C₁-C₄)alkenyl radical;
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical, or
R⁶ and R^{7Y} together are a bond, a —(C₁-C₄)alkanediyl or a (C₂-C₆)alkenediyl radical, and R^{7X} has the meaning given under;
R^{7X} and R^{7Y} are independently of one another an H atom, a (C₁-C₄)-alkyl, a (C₁-C₄)alkenyl radical, a (C₁-C₄)alkynyl-, or
R^{7X} and R^{7Y} together are a —(C₂-C₄)alkanediyl, or (C₂-C₆)alkenediyl radical, or
R^{7Y} and R⁶ together are a bond, a —(C₁-C₄)alkanediyl or a (C₂-C₆)alkenediyl radical, and R^{7X} has the meaning given above.

22. Compounds of the general formula (I) according to claim 21 in which
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7x} and R^{7Y} are independently of one another an H atom, a (C₁-C₄)-alkyl, a (C₁-C₄)alkenyl radical, a (C₁-C₄)alkynyl-.

23. Compounds of the general formula (I) according to claim 21 in which
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7x} and R^{7Y} are an H atom.

24. Compounds of the general formula (I) according to claim 21 in which
R⁶ is an H atom, a (C₁-C₄)alkyl, a (C₁-C₄)alkenyl, (C₁-C₄)alkynyl, a perfluoro(C₁-C₄)alkyl or a perfluoro(C₁-C₄)alkenyl radical;
R^{7x} is an H atom;
R^{7Y} is an H atom, a (C₁-C₄)-alkyl, a (C₁-C₄)alkenyl, or a (C₁-C₄)alkynyl radical.

25. Compounds of the general formula (I) according to claim 21, in which
R⁶ is an H atom.

26. Compounds of the general formula (I) according to claim 1 in which
A is 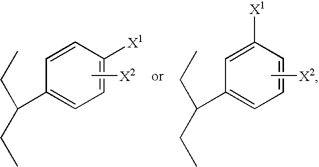
in which
X¹ is a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN,
X² is one or more groups on the phenyl ring and is independently of one another an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN.

27. Compounds of the general formula (I) according to claim 26,
in which
A is 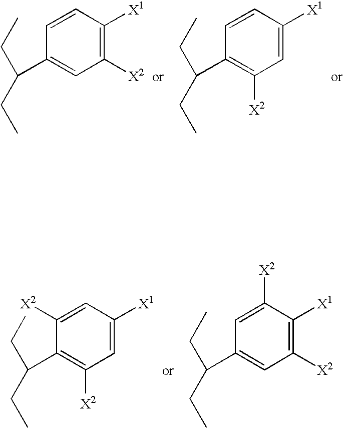
in which
X¹ is a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² is an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN.

28. Compounds of the general formula (I) according to claim 26,
in which
A is 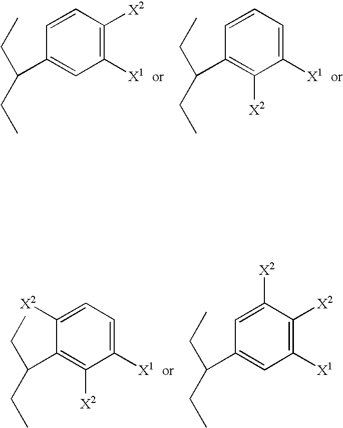
in which
X¹ is a halogen, OH, (C₁-C₄)alkyl, (C₁-C₄)alkyl-O, (C₃-C₆)cycloalkyl-O, (C₁-C₁₄)acyl-O, (C₁-C₄)alkenyl, (C₁-C₄)alkynyl, perfluoro(C₁-C₄)alkyl radical, —CHO or CN, and
X² is an H, halogen, OH, (C₁-C₄)alkyl, (C₁-C₄)alkyl-O, (C₁-C₄)alkenyl, (C₁-C₄)alkynyl, perfluoro(C₁-C₄)alkyl radical, —CHO or CN.

29. Compounds of the general formula (I) according to claim 26, in which
X¹ is an OH, (C₁-C₄)alkyl-O— or (C₃-C₆)cycloalkyl-O.

30. Compounds of the general formula (I) according to claim 26, in which
X¹ is a halogen, OH, (C₁-C₄)alkyl-, or (C₁-C₄)alkyl-O—.

31. Compounds of the general formula (I) according to claim 1
in which
R² and R⁴ are an H atom, and
R³ is an H atom or an F atom.

32. Compounds of the general formula (I) according to claim 1, as follows
1) 6-(4-hydroxyphenyl)-8,9-dihydro-7H-benzocyclohepten-2-ol
2) 6-(3-hydroxyphenyl)-8,9-dihydro-7H-benzocyclohepten-2-ol
3) 6-phenyl-8,9-dihydro-7H-benzocyclohepten-2-ol
4) 6-phenyl-5-methyl-8,9-dihydro-7H-benzocyclohepten-2-ol
5) 3-methoxy-9-methyl-8-phenyl-6,7-dihydro-5H-benzocycloheptene
6) 6-phenyl-5-ethyl-8,9-dihydro-7H-benzocyclohepten-2-ol
7) 9-ethyl-3-methoxy-8-phenyl-6,7-dihydro-5H-benzocycloheptene
8) 6-phenyl-5-propyl-8,9-dihydro-7H-benzocyclohepten-2-ol
9) 3-methoxy-8-phenyl-9-propyl-6,7-dihydro-5H-benzocycloheptene
10) 6-phenyl-5-butyl-8,9-dihydro-7H-benzocyclohepten-2-ol
11) 9-butyl-3-methoxy-8-phenyl-6,7-dihydro-5H-benzocycloheptene
12) 6-(4-hydroxyphenyl)-5-methyl-8,9-dihydro-7H-benzocyclohepten-2-ol
13) 3-methoxy-8-(4-methoxyphenyl)-9-methyl-6,7-dihydro-5H-benzocycloheptene
14) 4-(2-methoxy-5-methyl-8,9-dihydro-7H-benzocyclohepten-6-yl)phenol
15) 6-(4-methoxyphenyl)-5-methyl-8,9-dihydro-7H-benzocyclohepten-2-ol
16) 6-(3-hydroxyphenyl)-5-methyl-8,9-dihydro-7H-benzocyclohepten-2-ol
17) 5-ethyl-6-(4-hydroxyphenyl)-8,9-dihydro-7H-benzocyclohepten-2-ol
18) 9-ethyl-3-methoxy-8-(4-methoxyphenyl)-6,7-dihydro-5H-benzocycloheptene
19) 4-(5-ethyl-2-methoxy-8,9-dihydro-7H-benzocyclohepten-6-yl)phenol
20) 5-ethyl-6-(4-methoxyphenyl)-8,9-dihydro-7H-benzocyclohepten-2-ol
21) 6-(4-hydroxyphenyl)-5-propyl-8,9-dihydro-7H-benzocyclohepten-2-ol
22) 3-methoxy-8-(4-methoxyphenyl)-9-propyl-6,7-dihydro-5H-benzocycloheptene
23) 4-(2-methoxy-5-propyl-8,9-dihydro-7H-benzocyclohepten-6-yl)phenol
24) 6-(4-methoxyphenyl)-5-propyl-8,9-dihydro-7H-benzocyclohepten-2-ol
25) 6-(4-hydroxyphenyl)-5-butyl-8,9-dihydro-7H-benzocyclohepten-2-ol
26) 6-(3-hydroxyphenyl)-5-ethyl-8,9-dihydro-7H-benzocyclohepten-2-ol
27) 6-(4-hydroxyphenyl)-5-trifluoromethyl-8,9-dihydro-7H-benzocyclohepten-2-ol
28) 6-(4-hydroxyphenyl)-5-pentafluoroethyl-8,9-dihydro-7H-benzocyclohepten-2-ol
29) 6-(4-hydroxyphenyl)-5-vinyl-8,9-dihydro-7H-benzocyclohepten-2-ol
30) 3-methoxy-8-(4-methoxyphenyl)-9-vinyl-6,7-dihydro-5H-benzocycloheptene
31) 6-(3-hydroxyphenyl)-5-vinyl-8,9-dihydro-7H-benzocyclohepten-2-ol
32) 6-(4-hydroxyphenyl)-6,7,8,9-tetrahydro-5H-benzocyclohepten-2-ol
33) 6-(3-hydroxyphenyl)-6,7,8,9-tetrahydro-5H-benzocyclohepten-2-ol
34) 6-(2-hydroxyphenyl)-6,7,8,9-tetrahydro-5H-benzocyclohepten-2-ol
35) 6-(4-hydroxyphenyl)-5-methyl-6,7,8,9-tetrahydro-5H-benzocyclohepten-2-ol
36) 6-(4-hydroxyphenyl)-5-ethyl-6,7,8,9-tetrahydro-5H-benzocyclohepten-2-ol
37) 6-(4-hydroxyphenyl)-6-methyl-6,7,8,9-tetrahydro-5H-benzocyclohepten-2-ol
38) 2-methoxy-6-(4-methoxy-phenyl)-6-methyl-6,7,8,9-tetrahydro-5H-benzocycloheptene
39) 4-(2-methoxy-6-methyl-6,7,8,9-tetrahydro-5H-benzocyclohepten-6-yl)phenol
40) 6-(4-methoxyphenyl)-6-methyl-6,7,8,9-tetrahydro-5H-benzocyclohepten-2-ol
41) 6-(4-hydroxyphenyl)-6-ethyl-6,7,8,9-tetrahydro-5H-benzocyclohepten-2-ol
42) 6-ethyl-2-methoxy-6-(4-methoxyphenyl)-6,7,8,9-tetrahydro-5H-benzocycloheptene 43) 4-(6-ethyl-2-methoxy-6,7,8,9-tetrahydro-5H-benzocyclohepten-6-yl)phenol
44) 6-ethyl-6-(4-methoxyphenyl)-6,7,8,9-tetrahydro-5H-benzocyclohepten-2-ol
45) 6-(4-hydroxyphenyl)-6-vinyl-6,7,8,9-tetrahydro-5H-benzocyclohepten-2-ol
46) 2-methoxy-6-(4-methoxyphenyl)-6-vinyl-6,7,8,9-tetrahydro-5H-benzocycloheptene 47) 4-(2-methoxy-6-vinyl-6,7,8,9-tetrahydro-5H-benzocyclohepten-6-yl)phenol
48) 6-(4-methoxyphenyl)-6-vinyl-6,7,8,9-tetrahydro-5H-benzocyclohepten-2-ol
49) 6-(4-hydroxyphenyl)-4-(2-trimethylsilanyl-ethyl)-6,7,8,9-tetrahydro-5H-benzocyclohepten-2-ol
50) 6-(4-hydroxyphenyl)-4-(2-methyldimethoxysilanylethyl)-6,7,8,9-tetrahydro-5H-benzocyclohepten-2-ol
51) 6-(4-hydroxyphenyl)-4-(2-triethoxysilanylethyl)-6,7,8,9-tetrahydro-5H-benzocyclohepten-2-ol
52) 6-(4-hydroxyphenyl)-4-vinyl-6,7,8,9-tetrahydro-5H-benzocyclohepten-2-ol
53) 3-hydroxy-8-(4-hydroxyphenyl)-6,7,8,9-tetrahydro-5H-benzocyclohepten-1-carbaldehyde
54) 3-hydroxy-8-(4-hydroxy-phenyl)-6,7,8,9-tetrahydro-5H-benzocyclohepten-1-carbonitrile
55) 6-(4-hydroxyphenyl)-4-ethyl-6,7,8,9-tetrahydro-5H-benzocyclohepten-2-ol
56) 6-(4-hydroxyphenyl)-4-(2-fluoroethyl)-6,7,8,9-tetrahydro-5H-benzocyclohepten-2-ol
57) 6-(4-hydroxyphenyl)-4-chloro-6,7,8,9-tetrahydro-5H-benzocyclohepten-2-ol
58) 6-(4-hydroxyphenyl)-5-methylene-6,7,8,9-tetrahydro-5H-benzocyclohepten-2-ol
59) 6-(4-hydroxyphenyl)-5,5-spirooxirane-6,7,8,9-tetrahydro-5H-benzocyclohepten-2-ol
60) 6-(4-hydroxyphenyl)-5,5-spirocyclopropyl-6,7,8,9-tetrahydro-5H-benzocyclohepten-2-ol
61) 5-ethyl-6-(4-hydroxyphenyl)-6-methyl-6,7,8,9-tetrahydro-5H-benzocyclohepten-2-ol
62) 5-ethyl-6-(4-hydroxyphenyl)-6-ethyl-6,7,8,9-tetrahydro-5H-benzocyclohepten-2-ol
63) 5-hydroxy-2-(2-hydroxy-8,9-dihydro-7H-benzocyclohepten-6-yl)benzonitrile
64) 6-(2-chloro-4-hydroxyphenyl)-8,9-dihydro-7H-benzocyclohepten-2-ol
65) 3-fluoro-6-(4-hydroxyphenyl)-8,9-dihydro-7H-benzocyclohepten-2-ol
66) 6-(3-fluoro-4-hydroxyphenyl)-8,9-dihydro-7H-benzocyclohepten-2-ol
67) 6-(2-fluoro-4-hydroxyphenyl)-8,9-dihydro-7H-benzocyclohepten-2-ol
68) 3-fluoro-4-(2-methoxy-8,9-dihydro-7H-benzocyclohepten-6-yl)-phenol
69) 6-(2,3-difluoro-4-hydroxyphenyl)-8,9-dihydro-7H-benzocyclohepten-2-ol
70) 6-(2-chloro-3-fluoro-4-hydroxyphenyl)-8,9-dihydro-7H-benzocyclohepten-2-ol
71) 6-(2,5-difluoro-4-hydroxyphenyl)-8,9-dihydro-7H-benzocyclohepten-2-ol
72) 6-(3,5-difluoro-4-hydroxyphenyl)-8,9-dihydro-7H-benzocyclohepten-2-ol
73) 6-(2,6-difluoro-4-hydroxyphenyl)-8,9-dihydro-7H-benzocyclohepten-2-ol
74) 6-(3-chloro-4-hydroxyphenyl)-8,9-dihydro-7H-benzocyclohepten-2-ol
75) 6-(2-methyl-4-hydroxyphenyl)-8,9-dihydro-7H-benzocyclohepten-2-ol
76) 6-(2-ethyl-4-hydroxyphenyl)-8,9-dihydro-7H-benzocyclohepten-2-ol
77) 6-(2-vinyl-4-hydroxyphenyl)-8,9-dihydro-7H-benzocyclohepten-2-ol
78) 6-(2-trifluoromethyl-4-hydroxyphenyl)-8,9-dihydro-7H-benzocyclohepten-2-ol
79) 6-(2-chloro-4-hydroxyphenyl)-5-methyl-8,9-dihydro-7H-benzocyclohepten-2-ol
80) 3-fluoro-6-(4-hydroxyphenyl)-5-methyl-8,9-dihydro-7H-benzocyclohepten-2-ol
81) 6-(3-fluoro-4-hydroxyphenyl)-5-methyl-8,9-dihydro-7H-benzocyclohepten-2-ol
82) 6-(2-fluoro-4-hydroxyphenyl)-5-methyl-8,9-dihydro-7H-benzocyclohepten-2-ol
83) 6-(2,3-difluoro-4-hydroxyphenyl)-5-methyl-8,9-dihydro-7H-benzocyclohepten-2-ol
84) 6-(2-chloro-3-fluoro-4-hydroxyphenyl)-5-methyl-8,9-dihydro-7H-benzocyclohepten-2-ol
85) 6-(2,5-difluoro-4-hydroxyphenyl)-5-methyl-8,9-dihydro-7H-benzocyclohepten-2-ol
86) 6-(3,5-difluoro-4-hydroxyphenyl)-5-methyl-8,9-dihydro-7H-benzocyclohepten-2-ol
87) 6-(2,6-difluoro-4-hydroxyphenyl)-5-methyl-8,9-dihydro-7H-benzocyclohepten-2-ol
88) 6-(3-chloro-4-hydroxyphenyl)-5-methyl-8,9-dihydro-7H-benzocyclohepten-2-ol
89) 6-(2-methyl-4-hydroxyphenyl)-5-methyl-8,9-dihydro-7H-benzocyclohepten-2-ol
90) 6-(2-ethyl-4-hydroxyphenyl)-5-methyl-8,9-dihydro-7H-benzocyclohepten-2-ol
91) 6-(2-vinyl-4-hydroxyphenyl)-5-methyl-8,9-dihydro-7H-benzocyclohepten-2-ol
92) 6-(2-trifluoromethyl-4-hydroxyphenyl)-5-methyl-8,9-dihydro-7H-benzocyclohepten-2-ol
93) 5-hydroxy-2-(2-hydroxy-5-methyl-8,9-dihydro-7H-benzocyclohepten-6-yl)-benzonitrile
94) 6-(2-chloro-4-hydroxyphenyl)-5-ethyl-8,9-dihydro-7H-benzocyclohepten-2-ol
95) 5-ethyl-3-fluoro-6-(4-hydroxyphenyl)-8,9-dihydro-7H-benzocyclohepten-2-ol
96) 5-ethyl-6-(3-fluoro-4-hydroxyphenyl)-8,9-dihydro-7H-benzocyclohepten-2-ol
97) 6-(2-fluoro-4-hydroxyphenyl)-5-ethyl-8,9-dihydro-7H-benzocyclohepten-2-ol
98) 6-(2,3-difluoro-4-hydroxyphenyl)-5-ethyl-8,9-dihydro-7H-benzocyclohepten-2-ol
99) 6-(2-chloro-3-fluoro-4-hydroxyphenyl)-5-ethyl-8,9-dihydro-7H-benzocyclohepten-2-ol
100) 6-(2,5-difluoro-4-hydroxyphenyl)-5-ethyl-8,9-dihydro-7H-benzocyclohepten-2-ol
101) 6-(3,5-difluoro-4-hydroxyphenyl)-5-ethyl-8,9-dihydro-7H-benzocyclohepten-2-ol
102) 6-(2,6-difluoro-4-hydroxyphenyl)-5-ethyl-8,9-dihydro-7H-benzocyclohepten-2-ol
103) 6-(3-chloro-4-hydroxyphenyl)-5-ethyl-8,9-dihydro-7H-benzocyclohepten-2-ol
104) 6-(2-methyl-4-hydroxyphenyl)-5-ethyl-8,9-dihydro-7H-benzocyclohepten-2-ol
105) 6-(2-ethyl-4-hydroxyphenyl)-5-ethyl-8,9-dihydro-7H-benzocyclohepten-2-ol
106) 6-(2-vinyl-4-hydroxyphenyl)-5-ethyl-8,9-dihydro-7H-benzocyclohepten-2-ol
107) 6-(2-trifluoromethyl-4-hydroxyphenyl)-5-ethyl-8,9-dihydro-7H-benzocyclohepten-2-ol
108) 5-hydroxy-2-(2-hydroxy-5-ethyl-8,9-dihydro-7H-benzocyclohepten-6-yl)-benzonitrile
109) 5-hydroxy-2-(2-hydroxy-5-propyl-8,9-dihydro-7H-benzocyclohepten-6-yl)-benzonitrile
110) 6-(2-chloro-4-hydroxyphenyl)-5-propyl-8,9-dihydro-7H-benzocyclohepten-2-ol
111) 3-fluoro-6-(4-hydroxyphenyl)-5-propyl-8,9-dihydro-7H-benzocyclohepten-2-ol
112) 6-(3-fluoro-4-hydroxyphenyl)-5-propyl-8,9-dihydro-7H-benzocyclohepten-2-ol
113) 6-(2-fluoro-4-hydroxyphenyl)-5-propyl-8,9-dihydro-7H-benzocyclohepten-2-ol
114) 6-(2,3-difluoro-4-hydroxyphenyl)-5-propyl-8,9-dihydro-7H-benzocyclohepten-2-ol
115) 6-(2-chloro-3-fluoro-4-hydroxyphenyl)-5-propyl-8,9-dihydro-7H-benzocyclohepten-2-ol
116) 6-(2,5-difluoro-4-hydroxyphenyl)-5-propyl-8,9-dihydro-7H-benzocyclohepten-2-ol
117) 6-(3,5-difluoro-4-hydroxyphenyl)-5-propyl-8,9-dihydro-7H-benzocyclohepten-2-ol
118) 6-(2,6-difluoro-4-hydroxyphenyl)-5-propyl-8,9-dihydro-7H-benzocyclohepten-2-ol
119) 6-(3-chloro-4-hydroxyphenyl)-5-propyl-8,9-dihydro-7H-benzocyclohepten-2-ol
120) 6-(4-hydroxy-2-methylphenyl)-5-propyl-8,9-dihydro-7H-benzocyclohepten-2-ol
121) 6-(2-ethyl-4-hydroxyphenyl)-5-propyl-8,9-dihydro-7H-benzocyclohepten-2-ol
122) 6-(4-hydroxy-2-vinylphenyl)-5-propyl-8,9-dihydro-7H-benzocyclohepten-2-ol
123) 6-(4-hydroxy-2-trifluoromethylphenyl)-5-propyl-8,9-dihydro-7H-benzocyclohepten-2-ol
124) 6-(2-chloro-4-hydroxyphenyl)-6-methyl-6,7,8,9-tetrahydro-5H-benzocyclohepten-2-ol
125) 3-fluoro-6-(4-hydroxyphenyl)-6-methyl-6,7,8,9-tetrahydro-5H-benzocyclohepten-2-ol
126) 6-(3-fluoro-4-hydroxyphenyl)-6-methyl-6,7,8,9-tetrahydro-5H-benzocyclohepten-2-ol
127) 6-(2-fluoro-4-hydroxyphenyl)-6-methyl-6,7,8,9-tetrahydro-5H-benzocyclohepten-2-ol
128) 6-(2,3-difluoro-4-hydroxyphenyl)-6-methyl-6,7,8,9-tetrahydro-5H-benzocyclohepten-2-ol
129) 6-(2-chloro-3-fluoro-4-hydroxyphenyl)-6-methyl-6,7,8,9-tetrahydro-5H-benzocyclohepten-2-ol
130) 6-(2,5-difluoro-4-hydroxyphenyl)-6-methyl-6,7,8,9-tetrahydro-5H-benzocyclohepten-2-ol
131) 6-(3,5-difluoro-4-hydroxyphenyl)-6-methyl-6,7,8,9-tetrahydro-5H-benzocyclohepten-2-ol
132) 6-(2,6-difluoro-4-hydroxyphenyl)-6-methyl-6,7,8,9-tetrahydro-5H-benzocyclohepten-2-ol
133) 6-(3-chloro-4-hydroxyphenyl)-6-methyl-6,7,8,9-tetrahydro-5H-benzo-cyclohepten-2-ol
134) 6-(2-methyl-4-hydroxyphenyl)-6-methyl-6,7,8,9-tetrahydro-5H-benzocyclohepten-2-ol
135) 6-(2-ethyl-4-hydroxyphenyl)-6-methyl-6,7,8,9-tetrahydro-5H-benzocyclohepten-2-ol
136) 6-(2-vinyl-4-hydroxyphenyl)-6-methyl-6,7,8,9-tetrahydro-5H-benzocyclohepten-2-ol
137) 6-(2-trifluoromethyl-4-hydroxyphenyl)-6-methyl-6,7,8,9-tetrahydro-5H-benzocyclohepten-2-ol
138) 5-hydroxy-2-(2-hydroxy-6-methyl-6,7,8,9-tetrahydro-5H-benzocyclohepten-6-yl)benzonitrile
139) 6-(2-chloro-4-hydroxyphenyl)-6-ethyl-6,7,8,9-tetrahydro-5H-benzocyclohepten-2-ol
140) 3-fluoro-6-(4-hydroxyphenyl)-6-ethyl-6,7,8,9-tetrahydro-5H-benzocyclohepten-2-ol
141) 6-(3-fluoro-4-hydroxyphenyl)-6-ethyl-6,7,8,9-tetrahydro-5H-benzocyclohepten-2-ol
142) 6-(2-fluoro-4-hydroxyphenyl)-6-ethyl-6,7,8,9-tetrahydro-5H-benzocyclohepten-2-ol
143) 6-(2,3-difluoro-4-hydroxyphenyl)-6-ethyl-6,7,8,9-tetrahydro-5H-benzocyclohepten-2-ol
144) 6-(2-chloro-3-fluoro-4-hydroxyphenyl)-6-ethyl-6,7,8,9-tetrahydro-5H-benzocyclohepten-2-ol
145) 6-(2,5-difluoro-4-hydroxyphenyl)-6-ethyl-6,7,8,9-tetrahydro-5H-benzocyclohepten-2-ol
146) 6-(3,5-difluoro-4-hydroxyphenyl)-6-ethyl-6,7,8,9-tetrahydro-5H-benzocyclohepten-2-ol
147) 6-(2,6-difluoro-4-hydroxyphenyl)-6-ethyl-6,7,8,9-tetrahydro-5H-benzocyclohepten-2-ol
148) 6-(3-chloro-4-hydroxyphenyl)-6-ethyl-6,7,8,9-tetrahydro-5H-benzo-cyclohepten-2-ol
149) 6-(2-methyl-4-hydroxyphenyl)-6-ethyl-6,7,8,9-tetrahydro-5H-benzocyclohepten-2-ol
150) 6-(2-ethyl-4-hydroxyphenyl)-6-ethyl-6,7,8,9-tetrahydro-5H-benzocyclohepten-2-ol
151) 6-(2-vinyl-4-hydroxyphenyl)-6-ethyl-6,7,8,9-tetrahydro-5H-benzocyclohepten-2-ol
152) 6-(2-trifluoromethyl-4-hydroxyphenyl)-6-ethyl-6,7,8,9-tetrahydro-5H-benzocyclohepten-2-ol
153) 5-hydroxy-2-(2-hydroxy-6-ethyl-6,7,8,9-tetrahydro-5H-benzocyclohepten-6-yl)benzonitrile
154) 6-(2-chloro-4-hydroxyphenyl)-6,7,8,9-tetrahydro-5H-benzocyclohepten-2-ol
155) 3-fluoro-6-(4-hydroxyphenyl)-6,7,8,9-tetrahydro-5H-benzocyclohepten-2-ol
156) 6-(3-fluoro-4-hydroxyphenyl)-6,7,8,9-tetrahydro-5H-benzocyclohepten-2-ol
157) 6-(2-fluoro-4-hydroxyphenyl)-6,7,8,9-tetrahydro-5H-benzocyclohepten-2-ol
158) 6-(2,3-difluoro-4-hydroxyphenyl)-6,7,8,9-tetrahydro-5H-benzocyclohepten-2-ol
159) 6-(2-chloro-3-fluoro-4-hydroxyphenyl)-6,7,8,9-tetrahydro-5H-benzocyclohepten-2-ol
160) 6-(2,5-difluoro-4-hydroxyphenyl)-6,7,8,9-tetrahydro-5H-benzocyclohepten-2-ol
161) 6-(3,5-difluoro-4-hydroxyphenyl)-6,7,8,9-tetrahydro-5H-benzocyclohepten-2-ol
162) 6-(2,6-difluoro-4-hydroxyphenyl)-6,7,8,9-tetrahydro-5H-benzocyclohepten-2-ol
163) 6-(3-chloro-4-hydroxyphenyl)-6,7,8,9-tetrahydro-5H-benzocyclohepten-2-ol
164) 6-(2-methyl-4-hydroxyphenyl)-6,7,8,9-tetrahydro-5H-benzocyclohepten-2-ol
165) 6-(2-ethyl-4-hydroxyphenyl)-6,7,8,9-tetrahydro-5H-benzocyclohepten-2-ol
166) 6-(2-vinyl-4-hydroxyphenyl)-6,7,8,9-tetrahydro-5H-benzocyclohepten-2-ol
167) 6-(2-trifluoromethyl-4-hydroxy-phenyl)-6,7,8,9-tetrahydro-5H-benzocyclohepten-2-ol
168) 5-hydroxy-2-(2-hydroxy-6,7,8,9-tetrahydro-5H-benzocyclohepten-6-yl)-benzonitrile
169) 5-allyl-6-(4-hydroxyphenyl)-8,9-dihydro-7H-benzocyclohepten-2-ol
170) 9-allyl-3-methoxy-8-(4-methoxyphenyl)-6,7-dihydro-5H-benzocycloheptene
171) 6-allyl-2-methoxy-6-(4-methoxyphenyl)-6,7,8,9-tetrahydrobenzocyclohepten-5-one
172) 6-allyl-2-methoxy-6-(4-methoxyphenyl)-6,7,8,9-tetrahydro-5H-benzocycloheptene
173) 6-allyl-6-(4-hydroxyphenyl)-6,7,8,9-tetrahydro-5H-benzocyclohepten-2-ol
174) 2-methoxy-6-(4-methoxyphenyl)-6,7,8,9-tetrahydrobenzocyclohepten-5-one
175) 2-methoxy-6-(4-methoxyphenyl)-6-propyl-6,7,8,9-tetrahydrobenzocyclohepten-5-one
176) 2-methoxy-6-(4-methoxyphenyl)-6-propyl-6,7,8,9-tetrahydro-5H-benzocycloheptene
177) 6-(4-hydroxyphenyl)-6-propyl-6,7,8,9-tetrahydro-5H-benzocyclohepten-2-ol
178) 5-allyl-6-(4-hydroxyphenyl)-6-methyl-6,7,8,9-tetrahydro-5H-benzocyclohepten-2-ol
179) 5-allyl-6-(4-hydroxyphenyl)-6-ethyl-6,7,8,9-tetrahydro-5H-benzocyclohepten-2-ol
180) 6-(4-hydroxyphenyl)-4-trifluoromethyl-6,7,8,9-tetrahydro-5H-benzocyclohepten-2-ol
181) 4,6-diethyl-6-(4-hydroxyphenyl)-6,7,8,9-tetrahydro-5H-benzocyclohepten-2-ol
182) 4-ethyl-6-(4-hydroxyphenyl)-6-methyl-6,7,8,9-tetrahydro-5H-benzocyclohepten-2-ol
183) 6-(3-hydroxyphenyl)-6-methyl-6,7,8,9-tetrahydro-5H-benzocyclohepten-2-ol
184) 6-(3-hydroxyphenyl)-5-butyl-6,7,8,9-tetrahydro-5H-benzocyclohepten-2-ol
185) 6-(4-hydroxyphenyl)-5-propyl-6,7,8,9-tetrahydro-5H-benzocyclohepten-2-ol
186) 6-(3-hydroxyphenyl)-5-propyl-6,7,8,9-tetrahydro-5H-benzocyclohepten-2-ol
187) 6-(4-hydroxyphenyl)-5-propenyl-8,9-dihydro-7H-benzocyclohepten-2-ol
188) 6-(4-hydroxyphenyl)-5,5-dimethyl-8,9-dihydro-5H-benzocyclohepten-2-ol
189) 6-(4-hydroxyphenyl)-5,5-dimethyl-6,7,8,9-tetrahydro-5H-benzocyclohepten-2-ol
190) 5-ethyl-6-(4-hydroxyphenyl)-5-methyl-8,9-dihydro-5H-benzocyclohepten-2-ol
191) 5,5-diethyl-6-(4-hydroxyphenyl)-8,9-dihydro-5H-benzocyclohepten-2-ol
192) 6-(4-hydroxyphenyl)-5-phenyl-8,9-dihydro-7H-benzocyclohepten-2-ol
193) 6-(4-hydroxyphenyl)-5-(3-methylthiophen-2-yl)-8,9-dihydro-7H-benzocyclohepten-2-ol

33. Pharmaceutical compositions which comprise at least one compound of the general formula I according to claim 1, where appropriate together with pharmaceutically acceptable excipients and carriers.

34. Compounds of the general formula I according to claim 1 for use as medicament.
